(19) United States
(12) Patent Application Publication
(10) Pub. No.: US 2024/0123022 A1
(43) Pub. Date: Apr. 18, 2024

(54) A STABLE ANTI-ANGIOGENIC AND ANTI-INFLAMMATORY PHARMACEUTICAL FORMULATION AND PHARMACEUTICAL COMBINATION FOR TREATMENT AND PREVENTION OF PSORIASIS

(71) Applicant: Ramakrishna Reddy ISANAKA, Hyderabad, Telangana (IN)

(72) Inventor: Ramakrishna Reddy ISANAKA, Hyderabad, Telangana (IN)

(21) Appl. No.: 18/547,372

(22) PCT Filed: Jun. 9, 2022
(86) PCT No.: PCT/IB2022/055386

§ 371 Date: Aug. 22, 2023

(30) Foreign Application Priority Data
Dec. 16, 2021 (IN) 202141058681

(51) Int. Cl. A61K 38/08; A61K 9/00; A61K 9/06; A61K 31/122; A61K 31/519; A61K 31/573; A61K 47/10; A61K 47/12; A61K 47/14; A61K 47/18; A61K 47/26; A61K 47/44; A61P 9/00; A61P 29/00; C07K 1/36

(52) U.S. Cl. CPC A61K 38/08; A61K 9/0019; A61K 9/06; A61K 31/122; A61K 31/519; A61K 31/573; A61K 47/10; A61K 47/12; A61K 47/14; A61K 47/18; A61K 47/183; A61K 47/26; A61K 47/44; A61P 9/00; A61P 29/00; C07K 1/36

(57) ABSTRACT

The present invention relates to a stable anti-angiogenic and anti-inflammatory pharmaceutical formulation and pharmaceutical combination for treatment and/or prevention of psoriasis. The present invention also relates to methods of preparation of such formulation and combination and uses thereof.

### FIELD OF THE INVENTION

The present invention relates to a stable anti-angiogenic and anti-inflammatory pharmaceutical formulation and pharmaceutical combination for treatment and prevention of psoriasis. The present invention also relates to methods of preparation of such formulation and combination and uses thereof.

### BACKGROUND OF THE INVENTION

Psoriasis is a chronic inflammatory disease, which affects skin and small joints. It has an autoimmune mechanism wherein autoantigen is not determined. It can affect 2-4% of the worldwide population. Most of the patients present psoriatic lesions on scalp, elbows and knees. While it affects people of all ages, disease onset is commonly between ages of 15-25. Upto 30% of people with psoriasis will also develop psoriatic arthritis. Histopathological markers of skin in psoriasis include infiltration of multiple immune cells, keratinocyte hyperplasia, activated mast cells and accentuated vascularity in the dermis.

In the past decade, many studies have drawn attention to comorbid conditions in psoriasis and examined epidemiological evidence, pathophysiological commonalities, and therapeutic implications for different comorbidities of psoriasis. Cardiovascular disease, obesity, diabetes, hypertension, dyslipidemia, metabolic syndrome, non-alcoholic fatty liver disease, cancer, anxiety, depression and inflammatory bowel disease have been found at higher prevalence in psoriasis patients compared to general population. Psoriasis is commonly associated with a prominent permeability barrier abnormality and excess VEGF production.

Pathogenesis: Psoriasis is a chronic inflammatory skin disease characterized by dysregulation of keratinocyte cell growth, local immune activation, local inflammation and altered microvascular structure. It shares immunologic and genetic features with other autoimmune inflammatory conditions such as inflammatory bowel disease, rheumatoid arthritis and multiple sclerosis, all of which involve Th1 and Th17 cells. In psoriatic cells homeostatic mechanisms are disrupted and balance between growth regulatory signals and receptor expression is altered. Various factors which play a key role in the pathogenesis are T cells, antigen presenting cells (APC's), keratinocytes, Langerhans' cells, macrophages, natural killer cells, an array of Th1 type cytokines, certain growth factors like vascular endothelial growth factor (VEGF), keratinocyte growth factor (KGF), and dendritic cells (DC) (FIG. 1).

Tyrosine kinases (TKs) are key components of signaling pathways that drive an array of cellular responses including proliferation, differentiation, migration and survival.

On the other hand, vascular alterations observed in lesions would not only be a consequence of the disease, but in fact a key component as it would promote skin inflammation through recruitment of leukocytes. An increased angiogenesis caused by psoriatic skin cells and the activation of endothelial cells through pro-inflammatory cytokines therefore forms bridge between the altered epidermis and the immunological component of this disease. This presents evidence of both the importance of angiogenesis in pathogenesis of psoriasis and its clinical implications.

There is no complete cure for this common and enigmatic disease, and unequivocal genes or antigens responsible for its occurrence worldwide are yet to be identified. Currently new therapies are in pipeline, including an anti-IL-23/anti-IL-22 fully humanized antibody, anti-TNF, anti-VEFG, small-molecule inhibitors of TKs are emerging as a novel class of therapy that may provide benefit in certain patient subsets. But results of clinical trials will only prove its efficacy as long term cure.

One of the pathogenetic mechanisms in psoriasis is represented by VEGF-induced angiogenesis. The psoriatic patients have high serum levels of VEGF and endothelial cell stimulating angiogenesis factor (ESAF) and the psoriatic severity is correlated with the VEGF serum levels. Therefore, the current therapies for psoriasis have two target points: the immune response and the inhibition of neo-angiogenesis factors.

WO2020053200 discloses Wnt5A peptides and derivatives thereof for use in treatment of psoriasis. The Wnt5A peptide comprises XADGXBEL, wherein XA is methionine (M) or norleucine, XB is cysteine (C) or alanine (A), and wherein said Wnt5A peptide has a t-butoxycarbonyl protecting group in amino acid position 1.

WO1995019375 discloses method of preventing or reducing severity of psoriasis in an individual comprising administering a peptide having substantially sequence of a T cell receptor present on surface of T cells mediating psoriasis and capable of causing an effect on the immune system to regulate the T cells.

WO2021112615 discloses composition containing peptide P1 to P5 or a compound thereof as a treatment agent for inflammatory disease induced by an autoimmune reaction. The peptides have sequences P1: LICPEKYCNKVHT; P2: YCNKVHTCRNG; P3: PREIVECCSTDKCNH; P4: HTCRNGENICF and P5: ENICFKRFYEGNLLGKRYPRGCA.

WO2019059476 discloses the peptides for inhibiting angiogenesis and the composition containing the peptides as an active ingredient can exhibit an excellent prophylactic or therapeutic effect on diseases caused by excessive angiogenesis. The peptides have a sequence Asn Lys Asn Phe Gly Tyr Asp Leu Tyr Arg and Ile His Gly Thr Tyr Lys Glu Leu Leu.

U.S. Pat. No. 9,487,560B2 is the Applicant's patent directed to a lytic peptide fragment consisting of amino acid sequence of FAKKFAKKFK, wherein the lytic peptide fragment inhibits angiogenesis.

Several VEGF inhibitors were clinically tested in several malignancies as a strategy for the prevention of angiogenesis and vascular leakage. Drugs like G6-31, bevacizumab, ramucirumab, tanibirumab, sunitinib and pazopanib inhibit VEGFR-1, VEGFR-2, VEGFR-3, and PDGFR are also used for psoriasis. The inhibitors of VEGFR used in cancer patients may also have positive clinical effects in some psoriatic patients, but are recommended to be used in topical forms to limit toxicities. Therapeutic interventions for psoriasis may exacerbate comorbid conditions and vice versa. Due to the wide range of comorbid conditions associated with psoriasis, comprehensive screening and treatment must be implemented to most effectively manage psoriasis patients. Further, a need for comprehensive screening and treatment must be recognized and addressed. Therefore, appropriate management of psoriasis must involve an integrated approach.

Many treatment options exist like topical agents; non-biologic systemic treatments frequently used for psoriasis include methotrexate, ciclosporin, hydroxycarbamide, fumarates such as dimethyl fumarate and retinoids. Methotrexate and ciclosporin are drugs that suppress the immune system and retinoids are synthetic forms of vitamin A. These agents are also regarded as first-line treatments for psoriatic erythroderma. However, these agents have potentially severe side-effects due to their immunosuppressive mechanisms and can severely flare psoriasis upon their discontinuation. Therefore, the current research is focused on identifying novel and safe pharmacological agent with potent anti-inflammatory effect.

The known treatment strategies of psoriasis target specific areas of immune system and employ biologics. Biologics are medicines made from substances found in living things. The biologics treat psoriasis and some of such biologics are close to FDA approval. These lab-made proteins or antibodies are injected into skin or bloodstream of a subject in need. The biologics block part of altered immune system inside the body thereby alleviating symptoms of psoriasis. The biologics work on psoriasis by one of the mechanisms such as by curbing T cells (a form of white blood cell); blocking a substance called tumor necrosis factor-alpha (TNF-alpha), one of the main messenger chemicals in the immune system; by stopping a family of immune system's chemical messengers called interleukins in a subject; or by binding to proteins that cause inflammation.

However, moderate to severe cases of psoriasis require stronger drugs that can alter autoimmune response driving the disease. Some, newer-generation biologic drugs, require intravenous (IV) infusions or, more commonly, injections which a subject in need may administer himself. Self-injections, though may seem daunting, are practical than going back and forth to healthcare provider when frequent administration is needed. Most of the biologics approved for treatment of plaque psoriasis or psoriatic arthritis are delivered subcutaneously (SC). Current therapies for instance Cosentyx®, Taltz® (ixekizumab) and Otezla® (apremilast) act through blocking inflammatory cytokines (e.g., IL-17, IL-23, TNF-α). However, angiogenesis is not target of these current therapies. The best efficacy offered by current biologics target IL-17 or IL-23 pathway. Currently, there are no targeted topical formulations.

While monotherapy with biologics is effective for many patients with psoriasis, some patients require combination therapy. Many psoriasis medications can cause problems like diarrhea, headaches, or higher chances of infections. Combining two therapies allows administration of two or more active agents in lower doses, lessening the side effects and possible harms. For example, combination therapy followed by less intense phototherapy can lower chances of skin cancer. Combination therapies improve efficacy of both conventional and newer agents and help in reducing toxicity of higher dosages when administered alone. However, arriving at a potential combination therapy faces challenges such as following two different guidelines and schedules, not all drugs are safe to combine, increase in cost more than a single treatment or require more medical visits, etc., for instance, combination of salicylic acid with UVB phototherapy makes the UVB less effective and combination of cyclosporine with psoralen plus ultraviolet A can increase chances of development of cancer in a subject.

Despite of all the efforts made till date, a need exists for improved and effective means of curing or ameliorating one or more symptoms of psoriasis. There is a need for anti-psoriatic therapy which is capable of targeting both the angiogenesis and inflammatory pathway. There is a need to develop novel agents for psoriasis. The novel formulations and combinations having high shelf life by virtue of long term storage stability are desired. The stable topical and injectable, subcutaneous or intravenous, formulations and combinations having improved efficacy and reduced risk of unwanted toxicities are desired.

The present invention therefore aims at providing a peptide, combination thereof and formulations thereof having potential to target angiogenesis and inflammatory pathway for treating and preventing psoriasis. Further, cost of production for the peptide-based therapy is likely to be less as compared to biologics. Thus, the invention offers room for competitive pricing and market share in both subcutaneous and topical forms.

### DISCLOSURE OF THE INVENTION

The present invention provides a synthetic and non-steroidal decapeptide IS217 of SEQ. ID NO 1 for treatment and/or prevention of one or more symptoms of psoriasis. The present invention provides stable pharmaceutical formulations of therapeutically effective amount of decapeptide of SEQ. ID NO 1 along with one or more suitable pharmaceutically acceptable agents, suitable carriers, diluents, vehicles, or excipients. The present invention also provides pharmaceutical combinations of decapeptide IS217 with anthralin and/or methotrexate for treatment and/or prevention of psoriasis. The present invention provides stable formulations and combinations of the decapeptide of SEQ. ID NO 1 suitable as injectables, for instance subcutaneous or intravenous, and as topical mode of administration. The present application further provides use of the pharmaceutical formulations and pharmaceutical combinations of decapeptide IS217 for treatment and/or prevention of psoriasis. The invention also describes methods for preparation of such stable formulations and combinations. The invention further provides methods of treatment and prevention of one or more symptoms of psoriasis by the stable formulations and combinations.

In one of the embodiments, the present invention provides a stable anti-angiogenic and anti-inflammatory pharmaceutical formulation for treating and/or preventing one or more symptoms of psoriasis, said formulation comprising a peptide of SEQ. ID NO 1 or peptide variant thereof in an amount of from 0.01 μg/ml to 10,000 μg/ml and one or more suitable pharmaceutically acceptable excipients.

In one of the embodiments, said one or more suitable pharmaceutically acceptable excipients in the formulation are selected from the group consisting of suitable carriers, diluents, vehicles, disintegrant, swelling agent, antioxidant, buffer, bacteriostatic agent, emollient, emulsifier, plasticizer, penetration enhancer, preservative, cryoprotectant, neutralizer, fragrance additives, dispersants, surfactants, binders and lubricants.

In an embodiment, the present invention provides that said peptide variant in the formulation is at least 80%, at least 85%, at least 90%, at least 95%, at least 97%, at least 99% identical to the SEQ. ID NO 1.

In an embodiment, the present invention provides that said formulation is suitable as injectable, preferably subcutaneous route of administration and/or topical mode of administration.

In one of the embodiments, the present invention provides that said formulation treats and/or prevents one or more symptoms of psoriasis such as hyperplasia, parakeratosis, red patches of skin covered with thick, silvery scales, small scaling spots, dry cracked skin that may bleed and/or itch, itching, burning or soreness, thickened pitted or ridged nails, swollen and stiff joints.

In another embodiment, the present invention provides a stable anti-angiogenic and anti-inflammatory pharmaceutical combination comprising a peptide of SEQ. ID NO 1 or peptide variant thereof in an amount from 0.01 μg/ml to 10,000 μg/ml and one or more other active agent.

In another embodiment the present invention provides a combination for treating and/or preventing one or more symptoms of psoriasis.

In another embodiment the present invention provides that said other active agent in the combination is selected from a group consisting of anthralin, betamethasone and methotrexate.

In a yet another embodiment the present invention provides that said one or more other active agent in the combination is in an amount of 0.01 μM to 50 μM.

In a yet another embodiment the present invention provides that said peptide variant in the combination is at least 80%, at least 85%, at least 90%, at least 95%, at least 97%, at least 99% identical to the SEQ. ID NO 1.

In a further embodiment, the present invention provides that said combination is suitable as injectable, preferably subcutaneous route of administration and/or topical mode of administration.

In a further embodiment, the present invention provides that said combination treats and/or prevents one or more symptoms of psoriasis such as hyperplasia, parakeratosis, red patches of skin covered with thick, silvery scales, small scaling spots, dry cracked skin that may bleed and/or itch, itching, burning or soreness, thickened pitted or ridged nails, swollen and stiff joints.

In one of the embodiments, the present invention provides a method of preparing stable anti-angiogenic and anti-inflammatory pharmaceutical formulation comprising the steps:
a) preparing an aqueous phase with required quantity of pharmaceutically acceptable excipients by suitable means and heating the aqueous phase to a suitable temperature;
b) preparing an oil phase with required quantity of pharmaceutically acceptable excipients by suitable means and heating the oil phase to a suitable temperature until a clear solution is obtained;
c) preparing a solution comprising peptide of SEQ. ID NO 1 or variant thereof with required quantity of pharmaceutically acceptable excipients by suitable means;
d) adding oil phase obtained in step ‘b’ to aqueous phase obtained in step ‘a’ under continuous stirring for about 30 minutes and continuously increasing the stirring speed when both the phases are at same temperature;
e) cooling solution obtained in step ‘d’ to a suitable temperature followed by addition of solution obtained in step ‘c’ under continuous stirring at increased speed;
f) adding required quantity of tea tree oil in solution obtained in step ‘e’ under continuous stirring and adding water until a clear homogenous cream is formed.

In a further embodiment the present invention provides that the method of preparing the formulation comprises packaging of said cream prepared in suitable containers.

In an optional embodiment the present invention provides that the method of preparing the formulation comprises sterilization by suitable sterilization methods before or after packaging of said cream in suitable containers.

In an embodiment the present invention provides that said suitable temperature in steps ‘a’ and ‘b’ of the method of preparing the formulation is 70° C. to 80° C.

In an embodiment the present invention provides that said suitable temperature in step ‘e’ of the method of preparing the formulation is 30° C. to 40° C.

In another embodiment the present invention provides that steps ‘a’ and ‘b’ in the method of preparing the formulation require stirring at about 600 rpm.

In another embodiment the present invention provides that said increasing the stirring speed in step ‘d’ of the method of preparing the formulation is about 1000 rpm.

In a yet another embodiment the present invention provides that said increased stirring speed in step ‘e’ of the method of preparing the formulation is about 2000 rpm.

In a still another embodiment the present invention provides that said pharmaceutically acceptable excipients in step ‘a’ of the method comprises glycerol and/or triethanolamine.

In an embodiment the present invention provides that said pharmaceutically acceptable excipients in step ‘b’ of the method of preparing the formulation comprises cetyl alcohol, stearic acid, beeswax, isopropyl myristate and/or mineral oil.

In an embodiment the present invention provides that said pharmaceutically acceptable excipients in step ‘c’ of the method of preparing the formulation comprises ethylenediamine tetra-acetic acid disodium salt and/or trehalose dihydrate.

In one of the embodiments, the present invention provides a method of preventing and inhibiting angiogenesis in a subject in need thereof, said method comprising administering to the subject a therapeutically effective amount of a formulation or a combination comprising a therapeutically effective amount of peptide of SEQ. ID NO 1 or peptide variant thereof wherein the formulation or combination inhibits vascular endothelial growth factor (VEGF) expression.

In another embodiment the present invention provides that in the method of preventing and inhibiting angiogenesis in a subject in need thereof, said therapeutically effective amount of peptide of SEQ. ID NO 1 or peptide variant thereof in said formulation or combination is 0.01 μg/ml to 10,000 μg/ml. In another embodiment the present invention provides that in the method of preventing and inhibiting angiogenesis in a subject in need thereof, said combination comprises one or more other active agent selected from the group consisting of anthralin, betamethasone and methotrexate in an amount of 0.01 μM to 50 μM. In another embodiment the present invention provides that in the method of preventing and inhibiting angiogenesis in a subject in need thereof, said formulation or said combination is administered to the subject as injectable, preferably by subcutaneous mode of administration and/or topical mode of administration.

In one of the embodiments, the present invention provides a method of preventing and inhibiting inflammation in a subject in need thereof, said method comprising administering to the subject a therapeutically effective amount of the formulation or combination of present invention, wherein said formulation or combination inhibits expression of one or more of inflammatory markers p38 kinases, IL-17, IL-23 and IL-20.

In another embodiment the present invention provides that in the method of preventing and inhibiting inflammation in a subject in need thereof, said therapeutically effective amount of peptide of SEQ. ID NO 1 or peptide variant thereof in said formulation or combination is 0.01 μg/ml to 10,000 μg/ml. In another embodiment the present invention provides that in the method of preventing and inhibiting inflammation in a subject in need thereof, said combination comprises one or more other active agent selected from a group consisting of anthralin, betamethasone and methotrexate in an amount of 0.01 μM to 50 μM. In another embodiment the present invention provides that in the method of preventing and inhibiting inflammation in a subject in need thereof, said formulation or said combination is administered to the subject as injectable, preferably by subcutaneous mode of administration and/or topical mode of administration.

In one of the embodiments, the present invention provides a method of treating and preventing one or more symptoms of psoriasis in an individual, comprising administering to the individual the formulation or combination of present invention, wherein the formulation or combination is capable of anti-angiogenesis and anti-inflammatory potential.

In one of the embodiments, the present invention provides use of a formulation or combination of present invention, for preparation of a medicament for preventing or reducing the severity of psoriasis in an individual.

In one of the embodiments, the present invention provides use of formulation or combination of present invention for prevention and/or treatment of one or more symptoms of psoriasis in a subject in need thereof, comprising administering to the subject therapeutically effective amount of said formulation or combination, wherein said formulation or combination is capable of anti-angiogenesis and anti-inflammatory potential.

In one of the embodiments, the present invention provides a method of manufacturing decapeptide of SEQ. ID NO 1 or peptide variant thereof comprising the steps:
a) synthesizing said decapeptide by coupling one amino acid at a time, starting from C-terminus amino acid which is attached to a solid resin via a linker group;
b) controlling coupling of step a) by varying de-protection time and reagents, wherein de-protection is performed twice;
c) drying and weighing peptide resin obtained after coupling last amino acid;
d) cleaving resin-bound peptide off said resin by trifluoroacetic acid to obtain crude peptide;
e) optionally processing crude peptide obtained in step d) by reverse phase chromatography and ion exchange to obtain solution of purified peptide;
f) optionally lyophilizing said solution of purified peptide for removal of residual solvents.

### DESCRIPTION OF THE DRAWINGS

FIG. 1 demonstrates representation of molecular events involving key mediators responsible for pathogenesis of psoriasis.

FIG. 2 demonstrates chemical structures of IS217.

FIG. 3 demonstrates evaluation of pro-apoptotic effect of IS217 in human keratinocytes cells (HaCaT) by Annexin-V staining after 48 hours of treatment. *** indicates significant difference between groups at p<0.001

FIG. 4 demonstrates effect of IS217 on pro-apoptotic cells (SubG0/G1 population) after 48 hours of treatment. ***: p<0.001, ** p<0.01 (significantly different from the control-untreated group) statistical differences between control and treatment groups were determined using Graph pad prism 4.0, One way ANOVA with Bonferroni's multiple comparison post-test.

FIG. 5 demonstrates effect of IS217 on Caspase-3 enzyme activity after 48 hours of treatment.

FIG. 6 demonstrates pro-apoptotic effect of IS217 by DNA fragmentation in human keratinocytes HaCaT cells after 48 hours of treatment.

FIG. 7 demonstrates inhibitory effect of IS217 on VEGF secretion in human umbilical vein endothelial cells (HUVEC) after 24 hours. *** indicates significant difference between groups at p<0.01.

FIG. 8 shows representative pictures of western blotting of targets P-VEGFR-1, P-VEGFR-2, P-VEGFR-3, VEGFR-1, VEGFR-2, VEGFR-3, NRP-1, MPO, Akt, P-Akt, ERK1/2, P-ERK1/2, MAPK, p-38, P-p-38, p-NRP1 and GAPDH by IS217 at 500 and 1000 μg/ml.

FIG. 9 shows densitometric analysis results of IS217 at 500 μg/mL and 1000 μg/mL.

FIG. 10 demonstrates angiogenic effect of IS217 by assessing its inhibitory effects on HUVEC endothelial cells tube formation. *** indicates significant difference between groups at p<0.01.

FIG. 11 demonstrates representative pictures showing effect of IS217 on tube formation in HUVEC cells.

FIG. 12 demonstrates anti-angiogenic potential of IS217 in HUVEC through cell migration. *** indicates significant difference between groups at p<0.01

FIG. 13 demonstrates mechanistic pathway of IS217 molecule based on in-vitro assays of anti-angiogenic potential in HUVEC and biomarkers estimation.

FIG. 14 demonstrates anti-inflammatory potential of IS217 in splenocytes by inhibition of secretion of IL-17 after 24 hours of treatment. ***: p<0.001, **: p<0.01, *: p<0.05 (significantly different from control-ConA treated group)

FIG. 15 demonstrates anti-inflammatory potential of IS217 in human monocytic cell lines THP-1 by inhibition of secretion of IL-23 after 24 hours of treatment. ***: p<0.001, **: p<0.01, *: p<0.05 (significantly different from control-LPS and IFN-γ treated group)

FIG. 16 demonstrates effect of IS217 on vital parameters in Imiquimod (IMQ) induced psoriasis model in Balb/c mice.

FIG. 17 demonstrates histopathological pictures of ear sections stained with hematoxylin and eosin (H and E) dye for epidermal ear thickness measurement.

FIG. 18 demonstrates gross pictures of Balb/c mice of different groups of IMQ-induced psoriasis model.

FIG. 19 demonstrates effect of IS217 on vital parameters in IMQ-induced psoriasis model in Balb/c mice in dose response study.

FIG. 20 shows gross pictures of Balb/c mice in IMQ induced ear inflammation model demonstrating dose response study of IS217.

FIG. 21 demonstrates representative histopathological pictures of ear sections of Balb/c mice in IMQ-induced ear inflammation model demonstrating dose response of IS217.

FIG. 22 shows gross pictures of C57BL/6 mice in TPA (12-O-Tetradecanoylphorbol-13-acetate) induced ear inflammation model demonstrating dose response of IS217.

FIG. 23 demonstrates representative histopathological pictures of ear sections of C57BL/6 mice in TPA induced ear inflammation model demonstrating dose response of IS217.

FIG. 24 demonstrates anti-psoriatic potential of IS217 alone and in combination with methotrexate and betamethasone on vital parameters in TPA-induced ear inflammation model in C57BL/6 mice. $$p<0.01 compared with normal control, **p<0.01 compared with disease control, *p<0.05 compared with disease control.

FIG. 25 demonstrates effect of topical prophylactic treatment with 1% IS217 on ear thickness in IMQ-induced psoriasis model in Balb/c mice.

FIG. 26 demonstrates effect of topical prophylactic treatment with 1% IS217 on psoriatic score in IMQ-induced psoriasis model in Balb/c mice.

FIG. 27(A-D) demonstrates effect of topical prophylactic treatment with 1% IS217 on skin homogenate biomarkers (IL-17, IL-23, TNF-α and VEGF-A) score in IMQ-induced psoriasis model in Balb/c mice.

FIG. 28(A-D) demonstrates effect of topical prophylactic treatment with 1% IS217, on ear homogenate biomarkers (IL-17, IL-23, TNF-α and VEGF-A) score in IMQ-induced psoriasis model in Balb/c mice.

FIG. 29(A-B) demonstrates effect of topical prophylactic treatment with 1% IS217, on serum homogenate biomarkers (IL-17 and IL-23) score in IMQ-induced psoriasis model in Balb/c mice.

FIG. 30 demonstrates histopathological pictures of skin and ear sections stained with H and E dye for inflammatory parameters and epidermal ear thickness measurement of Balb/c mice in IMQ-induced ear inflammation model demonstrating dose response of topical prophylactic treatment with 1% IS217.

FIG. 31 shows gross pictures of Balb/c mice in IMQ-induced ear inflammation model demonstrating dose response study of topical prophylactic treatment with 1% IS217.

FIG. 32 demonstrates effect of topical semi therapeutic treatment with 1% IS217 on ear thickness in IMQ-induced psoriasis model in Balb/c mice.

FIG. 33 demonstrates effect of topical semi therapeutic treatment 1% IS217 on psoriatic score in IMQ-induced psoriasis model in Balb/c mice.

FIG. 34(A-D) demonstrates effect of topical therapeutic treatment with 1% IS217 on-serum homogenate biomarkers (IL-17, IL-23, TNF-α and VEGF-A) score in IMQ-induced psoriasis model in Balb/c mice.

FIG. 35(A-D) demonstrates effect of topical semi therapeutic treatment with 1% IS217 on ear homogenate biomarkers (IL-17, IL-23, TNF-α and VEGF-A) score in IMQ-induced psoriasis model in Balb/c mice.

FIG. 36(A-B) demonstrates effect of topical semi therapeutic treatment with 1% IS217 on serum homogenate biomarkers (IL-17 and IL-23) score in Imiquimod (IMQ) induced psoriasis model in Balb/c mice.

FIG. 37 demonstrates effect of topical therapeutic treatment with 1% IS217 on ear thickness in IMQ-induced psoriasis model in Balb/c mice.

FIG. 38 demonstrates effect of topical therapeutic treatment with 1% IS217 on psoriatic score in IMQ-induced psoriasis model in Balb/c mice.

FIG. 39(A-D) demonstrates effect of topical therapeutic treatment with 1% IS217 on skin homogenate biomarkers (IL-17, IL-23, TNF-α and VEGF-A) score in IMQ-induced psoriasis model in Balb/c mice.

FIG. 40(A-D) demonstrates effect of topical therapeutic treatment with 1% IS217 on ear homogenate biomarkers (IL-17, IL-23, TNF-α and VEGF-A) score in IMQ-induced psoriasis model in Balb/c mice.

FIG. 41(A-B) demonstrates effect of topical therapeutic treatment with 1% IS217 on serum homogenate biomarkers (IL-17 and IL-23) score in IMQ-induced psoriasis model in Balb/c mice.

FIG. 42 demonstrates histopathological pictures of skin and ear sections stained with H and E dye for inflammatory parameters and epidermal ear thickness measurement of Balb/c mice in IMQ-induced ear inflammation model demonstrating dose response of topical semi therapeutic and therapeutic treatment with 1% IS217.

FIG. 43 shows gross pictures of Balb/c mice in IMQ-induced ear inflammation model demonstrating dose response study of topical semi therapeutic and therapeutic treatment 1% IS217.

FIG. 44 demonstrates effect of parenteral treatment IS217 through subcutaneously, on ear thickness in IMQ-induced psoriasis model in Balb/c mice.

FIG. 45 demonstrates effect of parenteral treatment IS217 through subcutaneously, on psoriatic score in IMQ-induced psoriasis model in Balb/c mice.

FIG. 46(A-D) demonstrates effect of parenteral treatment IS217 through subcutaneously, on skin homogenate biomarkers (IL-17, IL-23, TNF-α and VEGF-A) score in IMQ-induced psoriasis model in Balb/c mice.

FIG. 47(A-D) demonstrates effect of parenteral treatment IS217 through subcutaneously, on ear homogenate biomarkers (IL-17, IL-23, TNF-α and VEGF-A) score in IMQ-induced psoriasis model in Balb/c mice.

FIG. 48(A-B) demonstrates effect of parenteral treatment with IS217 through subcutaneously, on serum homogenate biomarkers (IL-17 and IL-23) score in IMQ-induced psoriasis model in Balb/c mice.

FIG. 49 demonstrates histopathological pictures of skin and ear sections stained with H and E dye for inflammatory parameters and epidermal ear thickness measurement of Balb/c mice in IMQ-induced ear inflammation model demonstrating dose response of parenteral treatment with IS217 through subcutaneously.

FIG. 50 shows gross pictures of Balb/c mice in IMQ-induced ear inflammation model demonstrating dose response study of parenteral treatment IS217 through subcutaneously.

FIG. 51 demonstrates mechanism of action of IS217 for psoriasis as established by inventor of present invention based on preclinical data.

FIG. 52 demonstrates percent inhibition (cytotoxicity) of MTX in combination with IS217.

FIG. 53 demonstrates percent cytotoxicity values corresponding to combinations of Anthralin with IS217.

FIG. 54 demonstrates CI values corresponding to combinations of Anthralin with IS217.

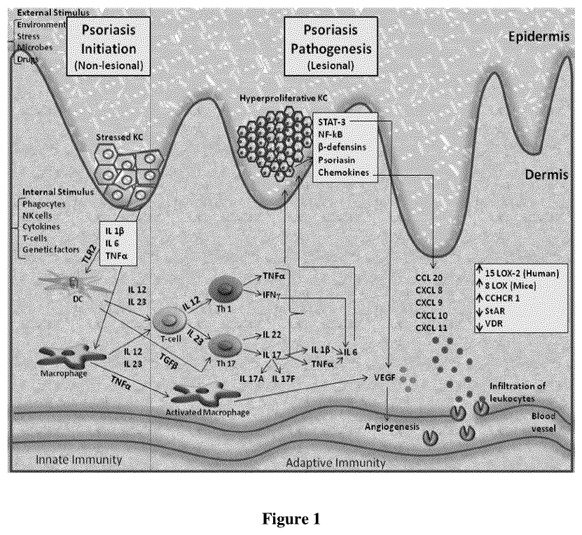

### DESCRIPTION OF THE INVENTION

The inventor of present invention developed lytic peptide sequences (U.S. Pat. No. 9,487,560 B2) with major components of antimicrobial defense systems of numerous species (AMPs) as an investigational lead molecule for treatment of various disease conditions. These synthetic lytic peptide fragments of full-length peptide are with capacity to modulate angiogenic activity in mammals. About 70 peptide sequences were identified as angiogenic lytic peptide, screened and studied. Finally, one peptide sequence “IS217” was identified as potent angiogenic lytic peptide (SEQ. ID NO 1). This peptide was also screened for anti-inflammatory activity which provides for treating treatment of disorders or diseases which involves angiogenesis and chronic inflammatory related disorders like psoriasis.

The present invention provides decapeptide IS217 of SEQ. ID NO 1 for treatment and/or ameliorating one or more symptoms of psoriasis. The IS217 is a non-biologic, non-steroidal, synthetic small peptide of 10 amino acids which has potential to arrest both angiogenesis and reduce inflammatory cytokines levels. The inventor of present invention tested the decapeptide against human keratinocyte cell lines (HaCaT), human umbilical vein endothelial cells (HUVEC) and endothelial cell lines to determine its anti-apoptotic effect and anti-angiogenic effect by inhibition of VEFG secretion with anti-proliferative activity. In-vitro evaluation on inhibition of topoisomerase II, tyrosine kinases and cytokinesis (IL-17, IL-20 and IL-23) inhibition was also determined. In-vivo animal studies have been carried to evaluate the efficacy of IS217 by using IMQ (imiquimod) and TPA mice models with different doses and routes. Molecular docking studies have also been carried out to complement the experimental results. This invention therefore relates to pharmaceutical formulations and combinations comprising IS217 (SEQ. ID NO 1) as an anti-psoriatic drug. The present invention also relates to methods of preparation of stable formulations and combinations of the decapeptide. The present invention further relates to use of such formulations and/or combinations for prevention and/or treatment of one or more symptoms of psoriasis.

The peptide, formulations and/or combinations thereof target the VEGFR-2 which is the major receptor for angiogenesis function. The in-vivo studies using IMQ animal model via subcutaneous administration showed high therapeutic effect of IS217 which are either comparable or even better to positive control group administrated with known drugs betamethasone and methotrexate.

The IS217 formulation and combinations of the present invention are suitable as injectables, for instance intravenous or subcutaneous, preferably as subcutaneous mode of administration and as topical mode of administration. The formulations both injectable and topical creams of the invention are easy to prepare and cost-effective.

The present invention demonstrates that IS217 targets VEGFR-2 by downregulating p³⁸ kinases and pro-inflammatory IL-23 and IL-17 secretion. The present invention provides pharmaceutical formulation and combination of the decapeptide to treat inflammatory skin diseases such as psoriasis as injectables and/or topical administration of the anti-angiogenic compound.

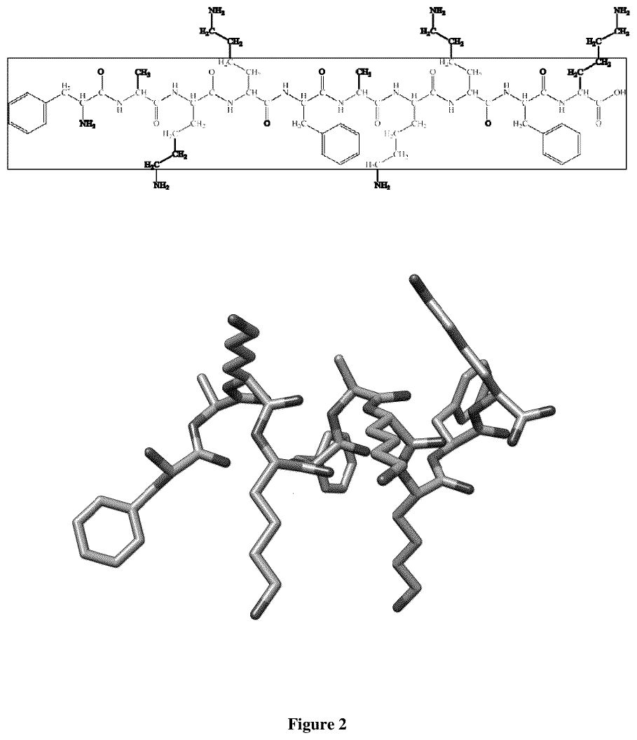

Synthetic peptide IS217 (FIG. 2)
Chemical composition: 10 amino acids
Chemical Formula: C₆₃H₉₉N₁₅O₁₁
Molecular mass: 1241.76
Molecular Weight: 1242.55 g/mol
Molecular three letter code: H-Phe-Ala-Lys-Lys-Phe-Ala-Lys-Lys-Phe-Lys-OH
(SEQ. ID No 1)
Molecular single letter code: H-F-A-K-K-F-A-K-K-F-K-OH (SEQ. ID No 1)
Molecular primary structure: linear peptide
Molecular secondary structure: β-sheet
Solubility: soluble in water

Although vascular re-modelling is a hallmark of many chronic inflammatory disorders, anti-vascular strategies to treat these conditions have received little attention to date. The inventor of present invention investigated effects of the synthetic peptide IS217 or variant thereof on endothelial cell function in vitro and its anti-inflammatory activity in different animal models. IS217 inhibited proliferation, migration, and tube formation by human umbilical vein endothelial cells in vitro. Whereas most studies performed to date with VEGFR TK inhibitors have focused on use of these compounds in oncology, the present invention investigated whether inhibition of VEGFR signaling might be useful for treatment of inflammatory skin disorders. Using an IMQ induced psoriasis mouse model of the chronic inflammatory skin disease, the inventor of present invention found that subcutaneous administration of IS217 effectively reduced psoriasis like inflammatory symptoms in the diseased skin of mice. Importantly, three major components of disease pathogenesis viz. infiltration of leukocytes, hyper-proliferation and abnormal differentiation of epidermal keratinocytes, and occurrence of vascular abnormalities were markedly improved following treatment with IS217 or variant thereof. These findings indicate that therapeutic intervention at level of vasculature could be sufficient to also reduce immune-mediated and epidermal components of the disease.

The formulation or combination of present invention can include variant of IS217 peptide. The variant is a functionally active variant and may be obtained by changing sequence of IS217 and is characterized by having a biological activity similar to that displayed by IS217 of SEQ. ID NO.1 from which the variant is derived. The variant includes anti-angiogenesis and anti-inflammatory ability of IS217. The functionally active variant of IS217 protein or domains thereof may be obtained by sequence alterations in sequence of IS217, wherein the peptide with the sequence alterations retains function of unaltered peptide. Such sequence alterations can include, but are not limited to, (conservative) substitutions, deletions, mutations and insertions. The variant can comprise at least 80% of the sequence of IS217, preferably at least 85%, still more preferably at least 90%, even more preferably at least 95% and most preferably at least 97%, 98% or 99%. The variant is derived from the IS217 by at least one amino acid substitution and/or deletion, wherein the functionally active variant has a sequence identity to IS217 of at least 80%, more preferably at least 85%, still more preferably at least 90%, even more preferably at least 95% and most preferably at least 97%, 98% or 99%. The variant of IS217 is functionally active in the context of the present invention, if the activity of the variant amounts to at least 10%, preferably at least 25%, more preferably at least 50%, even more preferably at least 70%, still more preferably at least 80%, especially at least 90%, particularly at least 95%, most preferably at least 99% of the activity of IS217 without sequence alteration. The activity of the variant may be determined or measured as described in the examples and then compared to that obtained for IS217 of the present invention.

The various embodiments of present invention will now herein below be described by means of examples. The following examples are provided solely to illustrate the present invention and are not intended to limit the scope of the invention, as described herein.

### Example 1: Manufacturing Synthetic Decapeptide IS217 (Fmoc Synthesis)

### 1.1: Synthesis and Cleavage:

The Fmoc process synthesizes the decapeptide, one amino acid at a time, starting from C-terminus amino acid which is attached to a solid resin via a linker group. The coupling procedure was a continuous process during which a series of wash steps were performed before and after addition of each amino acid derivative. The process was mainly controlled by de-protection time and reagents used for the reaction. For each cycle, de-protection was performed twice; first in about 20% piperidine in DMF for about five minutes. The solution was then drained out and a second de-protection was conducted for about additional 25 minutes to ensure the amino group is free for the coupling reaction. Coupling time was also a critical step for the Fmoc synthesis. Per Fmoc synthesis protocol, the coupling reaction with a specific Fmoc protected amino acid was controlled by the reaction time and the reaction time was at least 2 hours.

The peptide requires protection of their side chains during the Fmoc synthesis. The side chains of these amino acids were pre-protected by tBu, Boc, Trt, or Pbf groups. The cleavage of these side chain protection groups was completed using trifluoroacetic acid (TFA) solution, but not using piperidine which was used to remove the Fmoc group. As an example of a 300-mmol synthesis, the solid phase synthesis is performed on a peptide synthesizer and about 3 times excess of Fmoc protected amino acids were used for each coupling. The qualitative ninhydrin test was performed for each step to confirm completion of coupling. The ninhydrin test was performed by adding several drops of each of the ninhydrin test reagents to the sample of the peptide resin. The presence of free amino groups was indicated visually by presence of blue colour of beads, which represented a positive result. If the testing result was positive, a double coupling process was then performed. If it was determined that free amino groups are still present after the double coupling, the sequence was then acetylated using acetic anhydride to avoid undesirable reactions in the next cycle. After coupling the last amino acid (completion of the sequence) the peptide resin was dried and weighed. The weight of the peptide resin was an initial indicator of quality of the synthesis at that stage. The final peptidyl resin was obtained after final wash with methanol and dried under vacuum. The weight gain of the solid phase synthesis matched the synthetic scale as a fully protected peptide on the resin. With the substitution of 0.4-0.5 mmol/g of rink amide resin, a 300-mmol synthesis gives about 1.2 Kg of resin.

The resin-bound peptide was then cleaved off the resin with trifluoroacetic acid (TFA/TIPS/water (95/2.5/2.5%). The sidechain was also removed during this cleavage process. After 3-4 hours of reaction, the crude peptide was cut off, precipitated out, and washed with ethyl ether. Further vacuum dry gives an off-white powder (crude peptide). After cleavage, the crude peptide undergoes a series of processing steps, including purification by reverse phase chromatography and ion exchange. The resulting solution of purified peptide was then lyophilized to ensure complete removal of residual solvents.

Upon completion of the synthesis, the peptide acetate bulk drug substance was analyzed by gas chromatography for traces of residual solvents that may remain from the process. The specification of acetonitrile, dichloromethane and dimethylformamide in the final peptide acetate drug substance was <410 ppm, <600 ppm and <880 ppm, respectively. These established specifications provide assurance that residual solvents were removed during purification and were not present in the bulk drug substance with quantities over the specifications.

### 1.2 TFA (Trifluoroacetic Acid) Cleavage and Precipitation:

Cleavage of rink amide linker and AM (Aminomethyl) resin also requires acidic conditions. TFA is the primary reagent for such cleavage; however, since the cleavage involves multiple types of protection groups, the effective cleavage of these protection groups was accomplished by mixing TFA (95%) with triisopropylsilane (TIPS) (2.5%) and Water (2.5%). This mixture, of TFA, TIPS and water, called Cleavage Cocktail (it is 10 times of weight of peptidyl resin). After completion of last coupling de-protection was done twice with 20% Piperidine in Dimethyl formamide (DMF), 3 DMF washing, 2 DCM washing, 2 methanol washing, 2 diethyl ether washing and vacuum drying of peptidyl resin. Add cleavage cocktail reagents, one by one, in round bottom flask (RBF) and stir on magnetic stirrer. Take pre weighed peptidyl resin in another RBF and add cleavage cocktail into RBF containing peptidyl resin. Stir on magnetic stirrer for about 4 hours. The resulting raw peptide (peptide solution) was then filtered through G2 sintered funnel. Collected filtrates evaporate in Rota-evaporation, at 40° C. water bath temperature, until the volume gets reduced to 1/3 of its original volume. The resulting reduced volume of peptide solution transfer (drop by drop) into RBF containing chilled diethyl ether. The solution is left for mixing under stirring on a magnetic stirrer and after stirring time elapsed, the solution is left for settling down the precipitate in the bottom of the flask. Once precipitate is settled, the supernatant is decanted into an empty container. This process (precipitation of peptide) repeated 4 more times. Then isolation of solid crude peptide was done by centrifugation. The solid crude peptide is left for air drying overnight under fume hood. Powdery crude peptide kept in self-sealed covers, which are placed in PVC container and then stored at 2-8° C.

### 1.3: Purification by Reverse Phase Chromatography:

The purification equipment used was based on the principle of axial compression in which the chromatographic support is packed in a stainless-steel compression module. The stationary phase material was dedicated to the peptide, eliminating the possibility of cross-contamination. The choice of module used was based on amount of material to be processed. Solvents were delivered through pumps and the necessary gradients can be created with the pump integrated with an automatic gradient maker. The column can be used for purification of several syntheses of peptide until the column no longer meets the performance requirements.

The dried crude peptide (dissolved in 1000 ml of 20% Acetonitrile solution) was purified using reverse phase chromatography with a C-18 coated, 10 micron bead column The first purification buffer system included about 0.1% TFA in water (Mobile phase A) and 100% acetonitrile (ACN) (Mobile phase B). The flow rate was maintained with a linear gradient. All collected fraction send for analytical analysis. Pooled all fractions with purity ≥98% and evaporate it in rota-evaporation at 37° C. of water bath temperature, until the volume gets reduced to ⅓ of its original volume.

### 1.4: Counter Ion Exchange by Reverse Phase Chromatography:

The second in-process peptide solution was further purified and ion exchanged on the C-18 reverse phase high performance liquid chromatography (RP-HPLC) column. Mobile phases utilized for pre-wash were about 50 mM ammonium acetate buffer (pH 6, buffer A) and about 100% acetonitrile (ACN) (Buffer B). The mobile phases utilized for final ion exchange and purification were about 0.1% acetic acid (AcOH) in water (pH 3.5, Buffer A) and about 0.1% acetic acid (AcOH) in acetonitrile (ACN) (Buffer B). The flow rate was maintained at about 100 mL/min with a linear gradient of 10% to 70% Buffer B in about 60 minutes. Fractions with >98% purity were collected. The peptide solution was then lyophilized with a 12 hour (minimum) lyophilization cycle. This last purification process simultaneously converted the peptide drug substance into the acetate salt form. 1.5: Final lyophilisation: The collected reduced volume after roto evaporation was lyophilized under vacuum 50-100 millitorr and −70 to −50° C. (condenser temperature) for 48 hours to complete the peptide manufacturing process.

### 1.6: Disposition of Final Bulk Drug Substance:

Upon completion of the manufacturing process, the final bulk drug substance was held in the production until all analytical testing has been completed by quality control department, and quality assurance has reviewed the batch production record (including its related supporting documentation), and has released the drug substance. In order to perform the analytical testing, production sends requisitions to quality control through quality assurance. After getting report from quality control department, production handover the drug substance to the stores department for storage and further use in future.

### Example 2.1: Manufacture Procedure of IS217 Injectable Formulation

Each vial of IS217 API is reconstituted with about 0.9% sodium chloride solution for preparation of injectable formulation. Dissolution of the lyophilized powder is completed in less than 2 minutes.

Reconstitution of IS217 API lyophilized powder with 0.9% sodium chloride: The sodium chloride solution was injected into vial containing lyophilized IS217. The reconstituted solution was clear and colourless. The reconstituted solution was inspected visually for particulate matter and discoloration prior to administration.

Shelf life of IS217 API is about 2 years. Reconstituted solution of IS217 thus obtained is for single use only. Any unused medicinal product or waste material can be disposed of in accordance with local requirements. The reconstituted product thus obtained is preservative-free and is to be used immediately after preparation. The chemical and physical in-use stability of the reconstituted solution has been demonstrated for 24 hours at room temperature stored in original vial and/or syringe, with a total storage time for the reconstituted medicinal product not exceeding 24 hours prior to administration. Protecting the reconstituted medicinal product from light is not necessary.

### Example 2.2: Manufacture Procedure of 1% IS217 Cream Formulation

The method of preparing stable anti-angiogenic and anti-inflammatory pharmaceutical formulation comprises the following steps:
a) preparing an aqueous phase with required quantity of pharmaceutically acceptable excipients by suitable means and heating it to a suitable temperature;
b) preparing an oil phase with required quantity of pharmaceutically acceptable excipients by suitable means and heating it to a suitable temperature until a clear solution is obtained;
c) preparing a solution comprising peptide of SEQ. ID NO 1 or variant thereof with required quantity of pharmaceutically acceptable excipients by suitable means;
d) adding oil phase obtained in step ‘b’ to aqueous phase obtained in step ‘a’ when both the phases are at same temperature and increasing stirring speed under continuous stirring for about 30 minutes;
e) cooling solution obtained in step ‘d’ to a suitable temperature followed by addition of solution obtained in step ‘c’ under continuous stirring at increased speed;
f) adding required quantity of tea tree oil in solution obtained in step ‘e’ under continuous stirring and added water until a clear homogenous cream is formed.

The pharmaceutically acceptable excipients are one or more selected from the group consisting of carrier, disintegrant, swelling agent, lubricant, antioxidant, buffer, bacteriostatic agent, emollient, emulsifier, plasticizer, penetration enhancer, preservative, cryoprotectant, neutralizer, fragrance and diluent. It may further include one or more additives selected from the group consisting of dispersants, surfactants, binders or lubricants.

The carriers, excipients and diluents are selected from the group consisting of lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate and mineral oil can be used. The excipients include wetting agents, sweetening agents, fragrances, preservatives, mineral oil, stearic acid, cetyl alcohol, beeswax, glycerol, isopropyl myristate (PM), EDTA, trehalose dehydrate, triethanolamine, tea tree oil etc. in addition to water and optionally liquid paraffin, which are commonly used simple diluents, may be included. Formulations for parenteral administration include sterile aqueous solutions, non-aqueous solutions, suspensions, emulsions, freeze-dried preparations, and the like.

The pharmaceutical formulation can be administered to a subject in a conventional manner via intravenous, intraperitoneal, intramuscular, transdermal, topical and intradermal routes.

A preferred dosage of the formulation may vary depending on the condition and weight of the subject, the type and extent of the disease, the drug form, the route and duration of administration, and may be appropriately selected by those skilled in the art.

TABLE 1
Formulation of 1% IS217 cream

| | | Molecular | | |
|---|---|---|---|---|
| S. No. | Ingredients | weight (g/mole) | Role | Source |
| 1 | Peptide IS217 | 1242.60 | API | IN HOUSE |
| 2 | Mineral Oil | 452.36 | Emollient | Standard Reagents |
| | | | | (SR/0010316363) |
| 3 | Stearic Acid | 284.48 | Emulsifier | Kemphasol |
| | | | | (13PU12053) |
| 4 | Cetyl alcohol | 242.25 | Emulsifier | SDFCL |
| | | | | (38480K05) |
| 5 | Beeswax | 344.58 | Emulsifier | Central Drug House |
| | | | | PVT. LTD. (240817) |
| 6 | Glycerol | 92.09 | Plasticizer | Fischer Scientific |
| | | | | (24505) |
| 7 | Isopropyl | 270.45 | Penetration | Standard Reagents |
| | myristate (IPM) | | Enhancer | (SR/0010917268) |
| 8 | EDTA | 372.24 | Preservative | SDFCL(20093K05) |
| 9 | Trehalose | 378.33 | Cryoprotectant | Sigma Aldrich |
| | dihydrate | | | (90210-50G) |
| 10 | Triethanolamine | 149.19 | Neutralizer | SDFCL (40498L05) |
| 11 | Tea tree oil | 716.40 | Fragrance | Kazima perfumes |
| | | | | (MFG. December 2017) |
| 12 | Purified water | 18.00 | Aqueous | Millipore water |
| | | | Solvent | prepared in house |

### Preparation Procedure of 1% Cream Formulation of IS217:

### Aqueous Phase:

1. In a 1000 ml glass beaker, required quantity of purified water was added and placed on magnetic stirrer with temperature control. Required quantity of glycerol and triethanolamine was added slowly to the 1000 ml glass beaker and stirred at 600 RPM until it reached 75° C. temperature.

### Oil Phase:

2. On a separate magnetic stirrer with temperature control, a 100 ml glass beaker was placed and about required quantity of cetyl alcohol, stearic acid, beeswax, IPM and mineral oil were added and allowed to heat at 75° C. temperature until a clear solution was formed.
3. On another separate magnetic stirrer with temperature control, in 50 ml beaker of required quantity of millipore water, IS217, Ethylenediamine tetra-acetic acid disodium salt (EDTA) and Trehalose dihydrate were added and stirred with a glass rod.
4. When both the phases (aqueous and oil) are at same temperature i.e., 75° C., solution obtained in step-2 was added to solution obtained in step-1, 600 RPM increased to 1000 RPM and stirred continuously for about 30 minutes.
5. Step-3 added to the step-4, after step-4 is cooled to 37° C. and continue stirring at 2000 RPM.
6. Added about required quantity of tea tree oil under continuous stirring and added remaining water until a clear homogenous cream was formed and the cream prepared was packed into 50 gm tubes.

The cream prepared can be packed in any other suitable containers fit for use as per known practices in the pharmaceutical production processes, to enable desired administration.

The stable cream formulation thus prepared may be sterilized by suitable sterilization methods known in the pharmaceutical production processes before or after packaging in suitable containers. The stable cream formulation is thus prepared in the aseptic processing technique known in the pharmaceutical production processes before or after packaging in suitable containers.

### Example 3: In Vitro Mechanistic Profiling of IS217 Molecule as Anti-Apoptotic Drug

Based on targets involved in pathophysiology of psoriasis, in-vitro studies were carried to understand key factors involved in mechanism of action of psoriasis. In-vitro model studies revealed a complex interaction of dendritic cells, epidermal keratinocytes, and infiltrated immune cells and their pro-inflammatory cytokines. Mechanisitic profiling was performed for anti-apoptotic effect in HaCaT cells and anti-angiogenic effect in HaCaT cells, HUVEC cells and endothelial cell lines, respectively. Further targets—cell free and cell-based assays which involved cytokinesis, interleukins and kinases were carried out.

### 3.1: Evaluation of Pro-Apoptotic Effect of IS217 in Human Keratinocytes (HaCaT) Cells by Annexin-V Externalization [1-3]

Pro-apoptotic effect of IS217 was evaluated in human keratinocytes (HaCaT) cells by using the Annexin-V externalization. In this study, cells were plated and incubated for about 24 hours by using dulbecco's modified eagle's medium (DMEM) with about 10% fetal bovine serum (FBS) and cells were sera starved using about 0.1% FBS for about 3 hours. HaCaT cells were treated with IS217 at about 0 hours and about 24 hours in concentration range of from about 100 μg/ml to about 5000 μg/ml and incubated for about 24 hours. Curcumin (1 μM-50 μM) was used as positive control. After about 48 hours of first treatment, cells were incubated with Annexin-V reagent and subsequently analyzed by flow cytometry (Guava technologies) for identification of cells population.

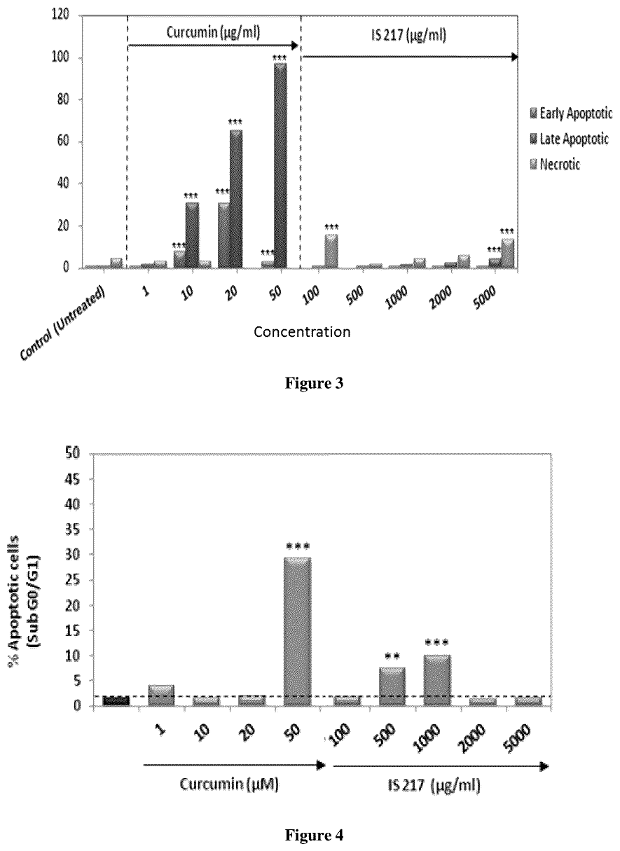

Results: When compared with control, IS217 showed no increase in early apoptotic cells and an increase in late apoptotic cells was observed by 1.7, 3.4 and 5.6 fold at concentrations of about 1000 μg/ml, 2000 μg/ml and 5000 μg/ml, respectively (FIG. 3). The same increase in necrotic cells was also observed by 3.6, 1.4 and 3.1 fold at concentration of about 100 μg/ml, 2000 μg/ml and 5000 μg/ml, respectively (FIG. 2). This study concludes that IS217 was able to show an increase in late apoptotic phase at highest concentrations but not in early phase.

### 3.2: Cell Cycle Assay (Pro-Apoptotic Effect) [1-3]

The in-vitro cell cycle assay was adapted to demonstrate the apoptotic potential of test formulation of IS217 in concentrations ranging from about 50 μg/mL to 2000 μg/mL. HaCaT cell lines were used as test system since it is widely used for studying anti-psoriatic effects. Curcumin dissolved in DMSO was taken as positive control for validation purpose. Apoptotic effect on HaCaT cell lines was detected by flow cytometry analysis.

Results: IS217 showed increased number of pro-apoptotic cells (Sub-G0/G1 population) by 1.2 fold, 5.0 fold, 6.8 fold and 1.1 fold at 100 μg/mL, 500 μg/mL, 1000 μg/mL and 5000 μg/mL, respectively as compared to control (untreated) levels (FIG. 4). Based on extent of increase in sub (G0/G1) phase, IS217 demonstrated higher extent of pro-apoptotic cells (FIG. 4).

### 3.3: Caspase-3 Activation Assay [4-6]

The study aimed to determine action of IS217 on the death mediator Caspase 3 which acts as dominant regulator during cell death induction. HaCaT cells were used as test system and in house validation assay was done by using Anthralin dissolved in DMSO (positive control).

Results: When HaCaT cells were treated with test item IS217 in concentration ranging from about 100 to 5000 μg/mL for about 24 hours and about 48 hours, findings from protein (Caspase 3) estimation through BCS colorimetric assay showed negative results against Caspase 3 enzyme activation (FIG. 5). Based on the results, IS217 did not lead to activation of Caspase 3 when compared to untreated cells at all the concentrations used for study (FIG. 5).

### 3.4: Evaluation of Pro-Apoptotic Effect of IS217 in Human Keratinocytes by Induction of DNA Fragmentation [7-9]

The study aimed to determine the apoptotic DNA fragmentation by IS217 in human keratinocytes. Here fluorescent method was used to quantify DNA with Hoechst 33258 dye and the BioTek Synergy HT multimode reader. Human keratinocytes were treated with the test item IS217 (from about 100 to about 5000 μg/ml) and stock solution of Anthralin 5 mM in DMSO used as positive control. Once within about 48 hours of incubation, re-treatment for test items done at every 24 hours. About 100 μl of supernatant containing fragmented DNA was aspirated and then incubated with 0.2 μg/ml of Hoechst 33258 dye for about 20 minutes and florescent was read at 360 ex/460 em using BioTek Synergy HT multimode reader.

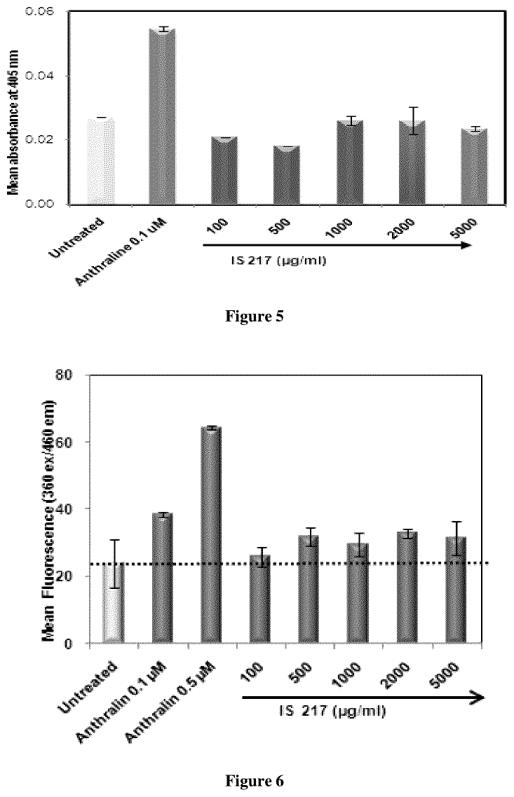

Results: The percentage DNA fragmentation inductions corresponding to each treatment with respect to the untreated cells were effective upto 100 to 2000 μg/ml (FIG. 6). The overall effect of IS217 on induction of DNA fragmentation w.r.t. un-treated in HaCaT cells ranged from 8.3 to 37.5% only (FIG. 6).

### 3.5: Tunel Assay [10-11]

Terminal deoxynucleotidyl transferase (TdT) dUTP Nick-End Labeling (TUNEL) assay has been designed to detect apoptotic cells that undergo extensive DNA degradation during the late stages of apoptosis.

Cytotoxic effect of IS217 on HaCaT cells was evaluated using TUNEL assay. Cells were treated with test item (IS217 of 100 μg/ml-5000 μg/ml) and positive controls like curcumin (10 μM and 20 μM), anthralin (1 μM) and betullininc acid (10 μg/ml, 20 μg/ml, 50 μg/ml). After about 48 hours of treatment, cells were analysed for the apoptotic effect via TUNEL assay. Absorbance was measured using ELISA reader (Table 1).

Results: No increase in apoptotic cells was demonstrated by IS217 as compared to control (untreated cells) (Table 1). These results demonstrate that IS217 did not exhibit any TUNEL positive population in tested concentration range.

TABLE 2
Effect of IS217 on pro-apoptotic cells (TUNEL positive
population) after 48 hours of treatment

| | | |
|---|---|---|
| | | % increase in TUNEL -Positive |
| | | cell population w.r.t. control |
| Sample | Concentration | (untreated cells) |

| | | | |
|---|---|---|---|
| IS 217 | 100 | μg/ml | −41−1 |
| | 500 | μg/ml | −25.0 |
| | 1000 | μg/ml | −20.4 |
| | 2000 | μg/ml | −25.4 |
| | 5000 | μg/ml | −40.7 |
| BA | 10 | μg/ml | 21.3 |
| | 20 | μg/ml | 48.2 |
| | 50 | μg/ml | 61.6 |
| Curcumin | 10 | μM | −72.2 |
| | 20 | μM | −76.1 |
| Anthralin | 1 | μM | −87.0 |

### Example 4: In-Vitro Mechanistic Studies of IS217 as Anti Angiogenic Drug

Anti-Angiogenic Effect

Enhanced angiogenesis is hallmark feature of psoriasis which is mediated via over expression of VEGF by keratinocytes [12]. HaCaT cells have been used as in-vitro tool to investigate anti-psoriatic compounds by estimating VEGF secretion. An inhibition of VEGF production indicates anti angiogenic potential. [13-16]

### 4.1: Anti-Angiogenic Effect in Human Keratinocytes (HaCaT) Cell Lines by Inhibition of VEFG Secretion

### 4.1.1: Inhibition of VEGF Secretion [13-16]

The study was to evaluate anti-angiogenic effect of IS217 in human keratinocytes (HaCaT) cells by determining percentage inhibition of VEGF secretion in HaCaT cells. Cells when treated with IS217 in concentrations of 0.1p g/mL-300 μg/mL, cell health was not good at 300 μg/mL. Hence, the concentration chosen for evaluating anti-angiogenic effect through ELISA was 0.1 μg/ml-100 μg/ml. curcumin, retinoic acids were taken as positive control for validation purpose.

Results: IS217 (0.1 μg/mL-100 μg/mL) demonstrated 1.3%-30.7% inhibition of VEGF secretion as compared to untreated cells. Positive controls (curcumin, retinoic acid at 1 μM) also led to inhibition of VEGF with 22.8%.

### 4.2: Tube Formation Assay [17]

This assay was performed to determine ability of various compounds to promote or inhibit tube formation. Compounds that are able to inhibit tube formation could be useful in various diseases, such as cancer, where tumors stimulate new blood vessel formation to receive oxygen and nutrients in order to grow beyond a relatively small size.

Human endothelial cells (EA.hy.926) grown in DMEM and about 10% FBS were used as test system to determine angiogenic effect of IS217 by assessing inhibitory effects of test item on endothelial cells tube formation.

Prior to the tube formation assay, test item of concentration ranging 1 μg/ml, 10 μg/ml, 100 μg/ml, 500 μg/ml and 1000 μg/ml were used to treat endothelial cells for its cytotoxicity effect to assess suitable concentration for tube formation assay. The cells corresponding to positive control group were treated with paclitaxel. Anthralin was also used as positive control for in house validation.

After about 24 hours of incubation in CO₂ incubator, 20 μl of 5 mg/ml of MTT was added to identify percentage cytotoxicty by biotek synergy HT plate reader. After that cells were processed for tube formation assay with selected concentrations of TI. The degree of angiogenesis progression was assessed based on visual pattern recognition of photomicrographs of EA.hy.926 cells.

Results: IS217 was observed to block endothelial tube formation in Ea.hy.926 cells at all the test concentrations ranging from 1 μg/ml to 1000 μg/m/l. Complete inhibition of endothelial morphogenesis on matrigel was obtained at concentration of 500 μg/ml of IS217.

### 4.3: In-Vitro Assessment of Apoptotic Effect of IS217 Through Induction of DNA Fragmentation in Human Endothelial Cell Line for Evaluation of Anti-Angiogenic Potential [18-19]

EA.hy926 cells, a well reported in vitro model to study the anti-angiogenic potential used for assessing apoptotic effect of IS217 through induction of DNA fragmentation. Cell conditions were maintained using DMEM, about 10% FBS, CO₂ incubator and serum starved using 1% FBS. The starved cells were treated with IS217 in the concentrations of 100 μg/ml, 500 μg/ml, 1000 μg/ml, 2000 μg/ml, 5000 μg/ml and incubated for about 48 hours. Anthralin in concentrations of 0.1 μM and 0.5 μM was used as positive control.

The fragmented DNA after lysis was precipitated using PEG/NaCl solution and incubated with 0.2 μg/ml of Hoechst 33258 dye, fluorescence was read at 360 ex/460 em using Biotek synergy HT multimode reader.

Results: IS217 showed a potent action on induction of DNA fragmentation only at concentrations ranging from 100 to 1000 μg/ml, at maximum concentration (1000 μg/ml) 24.53% of induction was noticed. But it was also observed that there was a significant decrease in percentage of induction of DNA fragmentation when cells treated with 2000 μg/ml and 5000 μg/ml.

### Example 5: In Vitro Assessment of Anti-Proliferative Effects of IS217 in Human Endothelial Cell Line for Evaluation of Anti-Angiogenic Potential [20-21]

The study aimed to determine the anti-angiogenic effect of IS217 in human endothelial cell line (EA.hy926) by assessing anti-proliferative effects of test item. Here MTT assay and BioTek Synergy HT multimode reader were used to determine the cell viability in test cells. Human endothelial cells were treated with the test item IS217 (1-5000 μg/ml) and anthralin 0.01 mM, 0.1 mM, and 0.5 mM, and paclitaxel 10 nM to 500 nM concentrations were used as positive controls. Once within about 72 hours of incubation, re-treatment was done for test items at every 24 hours in 3 additions. 150 μl of DMSO was added to aspirated supernatant and observed at 540 nm using BioTek Synergy HT plate reader.

Conclusions: Based on the results, it was concluded that percentage inhibition of FBS induced proliferation of endothelial cells corresponding to each treatment with respect to the untreated cells were effective upto 2000 μg/ml only and cytotoxic effect was observed at higher concentration 5000 μg/ml.

### Example 6: Evaluation of Anti-Angiogenic Potential of IS217 Through Cell Migration in Human Endothelial Cell Line [22-25]

The study was conducted to assess the anti-angiogenic potential of test item IS217 in human endothelial cell line (EA.hy926) through cell migration. Here MTT assay, BioTek Synergy HT multimode reader was used to determine suitable concentration for cell migration assay by identifying non cytotoxic concentration of test item. Whereas migration assay and Image J software was used for determination of the cell migration with test formulation.

For determination of non-cytotoxic concentrations, human endothelial cells were treated with the test item IS217 at concentration range from 0.1 μg/ml to 2 mg/ml with paclitaxel (10 nM) in DMEM and about 10% FBS as a positive control for non-cytotoxic concentrations with about 24 hours of incubation. 150 μl of DMSO was added to aspirated supernatant and observed at 540 nm using BioTek Synergy HT plate reader.

For determination of cell migration level, human endothelial cells were treated with the test item IS217 at concentration range from 0.1 μg/ml-1 mg/ml with paclitaxel (10 nM) as a positive control with about 24 hours of incubation. Later, images of wound were taken at 0 hour and 14 hours at three points in single well and distance of wound closure was measured using Image J Software.

Conclusions: Based on the results, IS217 was found to be non-cytotoxic at concentrations ranging from 0.1 μg/ml to 2 mg/ml on test cells as compared with controlled cells and it also demonstrated 24% to 66% percentage of inhibition in migration of test cells exhibited at concentrations ranging from 0.1 μg/ml to 1 mg/ml with respect to 0 hour control after 14 hours. Overall, this study demonstrated that IS217 exhibits better potential activity to inhibit migration of human endothelial cell line.

### Example 7: Anti-Angiogenic Effect in Human Umbilical Vein Endothelial (Huvec) Cells [27-44]

### 7.1: Cytotoxicity Assay in HUVEC Cell Lines

The assay was performed to evaluate cytotoxicity and inhibitory concentration (IC₅₀) for IS217 in HUVEC. Cells when treated with IS217 in concentrations of 0.01 μg/ml to 1000 μg/ml.

Results: All the doses ranging from 0.01-1000 μg/ml of IS217 were not cytotoxic (>30%) to HUVEC cells. IS217 exhibited dose dependent inhibition of VEGF stimulated HUVEC cells, although no inhibition for healthy unstimulated cells as seen in CC50 experiment. Hence the doses 100 to 1000 μg/mL were considered based on the dilution assays carried out.

### 7.2: Proliferation Assay in HUVEC Cell Lines

The study was performed to determine anti-angiogenic effect of IS217 in HUVEC cell line by assessing anti-proliferative effects of test item. Here XTT assay was used to determine the cell viability in test cells. HUVEC cells were treated with the test item IS217 (100-1000 μg/ml) and bevacizumab (1 mg/ml) concentrations was used as positive controls.

Conclusions: Based on the results, it was concluded that percentage inhibition of FBS induced proliferation of HUVEC cells corresponding to each treatment with respect to the untreated cells were effective upto 1000 μg/ml only and cytotoxic effect was observed at higher concentration of about 1000 μg/ml. IS217 (100 μg/ml-1000 μg/ml) demonstrated 21.25% to 31.08% of inhibition in VEGF induced cell proliferation compared to control.

### 7.3: Inhibition of VEGF Secretion

The study was conducted to evaluate anti-angiogenic effect of IS217 in HUVEC cells. Cells when treated with IS217 in concentrations of 0.01 μg/ml-1000 μg/ml. Bevacizumab (1 mg/mL) was taken as positive control for validation purpose.

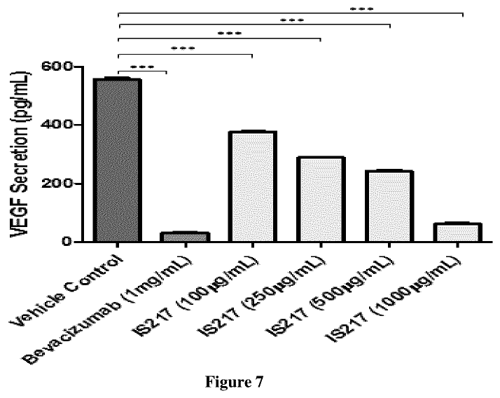

Results: IS217 (100 μg/ml to 1000 μg/ml) demonstrated 32.35% to 88.91% inhibition of VEGF secretion as compared to control. Positive control (bevacizumab) 1 mg/ml also led to inhibition of VEGF with 94.51% (FIG. 7).

### Example 8: Biomarker Estimation by Western Blotting Analysis

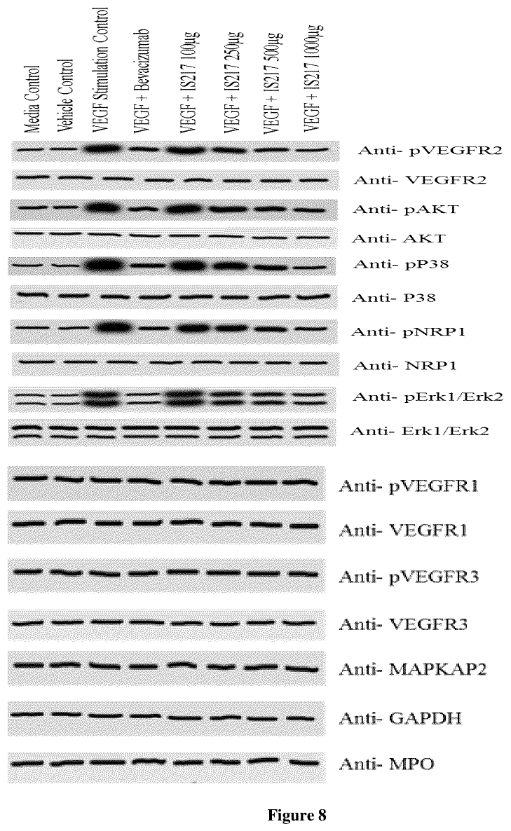

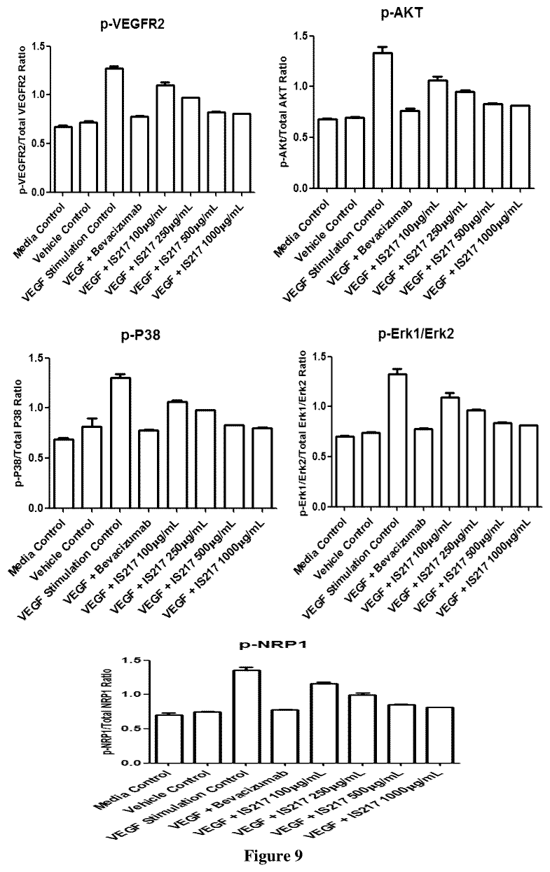

Targets screened are as follows: P-VEGFR-1, P-VEGFR-2, P-VEGFR-3, VEGFR-1, VEGFR-2, VEGFR-3, NRP-1, MPO, Akt, PAkt, ERK1/2, P-ERK1/2, MAPK, p-38, P-p-38, p-NRP1 and GAPDH. The protein levels of P-VEGFR-2, VEGFR-2, NRP-1, Akt, p-NRP1, P-Akt, ERK1/2, P-ERK1/2, and p-38, P-p-38, were downregulated by IS217 at 500 and 1000 μg/ml (FIG. 8). The densitometry analysis results of IS217 at 500 μg/ml and 1000 μg/ml demonstrated inhibition of VEGF secretion through as compared to bevacizumab (FIG. 9).

### Example 9: Tube Formation Assay

HUVEC cells were used as test system to determine angiogenic effect of IS217 by assessing the inhibitory effects of test item on endothelial cells tube formation. Prior to the tube formation assay, test item of concentration ranging 0.01 μg/ml to 1000 μg/ml were used to treat endothelial cells for its cytotoxicity effect to assess suitable concentration for tube formation assay. Hence the doses 100 μg/ml to 1000 μg/ml were considered further for tube formation assay. The cells corresponding to positive control group were treated with bevacizumab of concentration 1000 μg/ml. The degree of angiogenesis progression was assessed based on visual pattern recognition of photomicrographs of HUVEC cells.

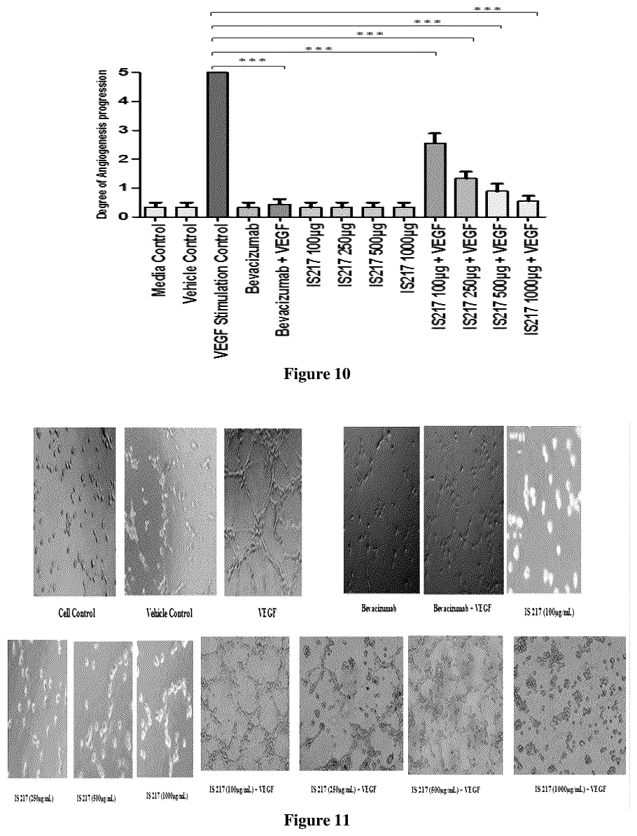

Results: IS217 was observed to block endothelial tube formation in HUVEC cells at all the test concentrations ranging from 100 μg/ml to 1000 μg/ml. Complete inhibition of endothelial morphogenesis on Matrigel was obtained at concentration of 1000 μg/ml of IS217 (Table 2, FIG. 10 and FIG. 11). IS217 inhibited VEGF induced tube formation in a dose dependent manner. IS217 (100 μg/ml to 1000 μg/ml) demonstrated 48.8% to 88.88% inhibition of tube formation as compared to VEGF stimulation control. Bevacizumab demonstrated 91.1% inhibition of tube formation as compared to VEGF stimulation control

TABLE 3
Tube formation assay results of IS217
Tube formation assay results for IS217

| SET | I | | | II | | | III | | | | |
|---|---|---|---|---|---|---|---|---|---|---|---|
| Fields | 1 | 2 | 3 | 1 | 2 | 3 | 1 | 2 | 3 | Avg | Std. Dev |

| | | | | | | | | | | | |
|---|---|---|---|---|---|---|---|---|---|---|---|
| Media Control | 0 | 1 | 0 | 0 | 1 | 0 | 0 | 1 | 0 | 0.33 | 0.5 |
| Vehicle Control | 1 | 0 | 0 | 1 | 0 | 1 | 0 | 0 | 0 | 0.33 | 0.5 |
| VEGF Stimulation | 5 | 5 | 5 | 5 | 5 | 5 | 5 | 5 | 5 | 5 | 0 |
| Control | | | | | | | | | | | |
| Bevacizumab | 0 | 1 | 0 | 1 | 0 | 1 | 0 | 0 | 0 | 0.33 | 0.5 |
| Bevacizumab + VEGF | 0 | 1 | 0 | 0 | 1 | 0 | 0 | 1 | 1 | 0.44 | 0.527 |
| IS 217 100 μg | 0 | 1 | 0 | 0 | 1 | 0 | 0 | 1 | 0 | 0.33 | 0.5 |
| IS 217 250 μg | 1 | 0 | 1 | 0 | 0 | 1 | 0 | 0 | 0 | 0.33 | 0.5 |
| IS 217 500 μg | 1 | 0 | 1 | 0 | 0 | 1 | 0 | 0 | 0 | 0.33 | 0.5 |
| IS 217 1000 μg | 0 | 0 | 1 | 0 | 1 | 0 | 1 | 0 | 0 | 0.33 | 0.5 |
| IS 217 100 μg + VEGF | 2 | 3 | 3 | 4 | 2 | 1 | 2 | 4 | 2 | 2.55 | 1.014 |
| IS 217 250 μg + VEGF | 1 | 1 | 3 | 1 | 2 | 1 | 1 | 1 | 1 | 1.33 | 0.707 |
| IS 217 500 μg + VEGF | 0 | 1 | 0 | 1 | 2 | 1 | 2 | 1 | 0 | 0.88 | 0.782 |
| IS 217 1000 μg + VEGF | 1 | 1 | 1 | 0 | 0 | 1 | 0 | 1 | 0 | 0.55 | 0.527 |

### Example 10: Migration Assay to Study Effect of IS217 on VEGF Stimulated HUVEC Migration

The study was conducted to assess the anti-angiogenic potential of test item IS217 in HUVEC cells through cell migration. Migration is a key property of live cells and critical for normal development, immune response, and disease processes such as cancer metastasis and inflammation. So, the inventor conducted further studies to test whether the test compound IS217 is having cell migratory behavior to prove as anti-angiogenic drug. Here XTT assay, BioTek Synergy HT multimode reader was used to determine the suitable concentration for cell migration assay by identifying non cytotoxic concentration of test item. Whereas migration assay and Image J software was used for determination of the cell migration with test formulation.

For determination of cell migration level, human endothelial cells were treated with the test compound IS217 at concentration range from about 100 μg/ml to 1000 mg/ml with bevacizumab 1000 μg/ml as a positive control with about 24 hours of incubation.

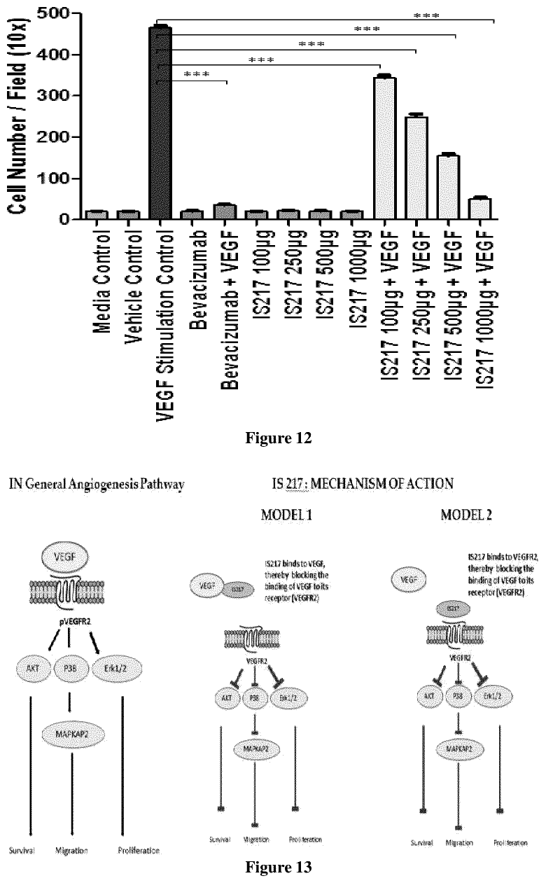

Conclusions: Based on the results, IS217 was found to be non-cytotoxic at concentrations ranging from 100 μg/ml to 1000 μg/ml. Different concentrations of IS217 (100, 250, 500, and 1000 μg/ml) without VEGF stimulation are compared with vehicle control. Various concentrations of IS217 (100, 250, 500 and 1000) μg/ml with VEGF (10 ng/ml) stimulation are compared with VEGF control and same for the positive control bevacizumab (1000 μg/L) control. Overall, the assay demonstrates that IS217 exhibit better potential activity to inhibit migration of HUVEC (FIG. 12). IS217 (100 μg/ml to 1000 μg/ml) demonstrated 26.17% to 89.08% inhibition of cell migration as compared to VEGF stimulation control.

Based on the above in-vitro assays of anti-angiogenic potential in HUVEC cells and biomarkers estimation, mechanistic pathway of IS217 molecule was determined (FIG. 13).

### Example 11: Evaluation of Anti-Inflammatory Activity of IS217 by IL-17 Inhibition in Splenocytes [45-48]

The study was conducted to assess anti-inflammatory potential of test item IS217 in murine splenocytes isolated from C57BL/6 mice for assessing percentage inhibition of IL-17 in splenocytes by using ELISA method. Test item IS217 of concentration ranging 10-5000 μg/ml was used to identify the non-cytotoxic effect for determining suitable concentration for IL-17 assay. Curcumin was taken as positive control for in-house validation.

For determining inhibitory activity against IL-17 secretion level, splenocytes cells were treated with the concentration range of 10 to 5000 μg/ml along with Con A stimulation (5 μg/ml), Con A alone treated cells were used as a positive control. After incubating for about 24 hours, IL-17 levels were estimated by ELISA corresponding to non-cytotoxic concentrations at 10 to 2000 μg/ml and absorbance of samples was determined by using ELISA reader.

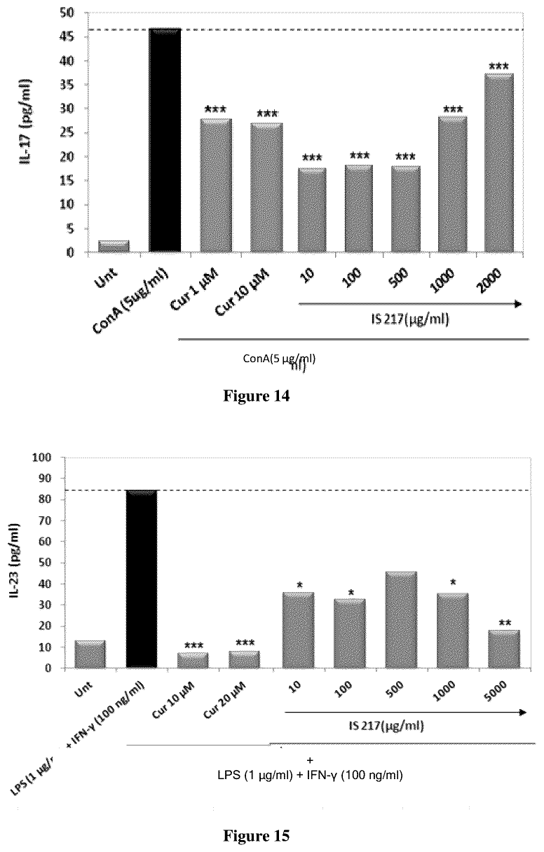

Conclusions: Based on the results, IS217 (10 μg/ml to 2 mg/ml) demonstrated 20% to 62.3% inhibition of IL-17 secretion as compared to control (ConA) levels (FIG. 14). Curcumin positive control also led to IL-17 inhibition. Treatment of HaCaT cells with curcumin at 1 μM and 10 μM resulted in 40.5% and 42.5% inhibition of IL-17 w.r.t. control (ConA) levels, respectively.

### Example 12: Evaluation of Anti-Inflammatory Potential of IS217 in Monocytes by Inhibition of IL-23 Secretion [48-49]

The study was conducted to assess anti-inflammatory potential of test item, IS217 in human monocytic cell line (THP-1) by assessing percentage inhibition of IL-23 in monocytes by using ELISA method.

Test item IS217 of concentration ranging 10 to 5000 μg/ml was used to assess its cytotoxic effect for determining suitable concentration for IL-23 assay. Curcumin was taken as positive control for in-house validation. For determining inhibitory activity against IL-23 secretion, human monocytic cell line (THP-1) was treated with concentration range of 10-5000 μg/ml with LPS (1 μg/ml) and IFN-γ. LPS (1 μg/ml) and IFN-γ (100 ng/ml) alone were taken as a positive control. After about 24 hours of incubation, IL-23 levels were estimated by ELISA in supernatants corresponding to non-cytotoxic concentrations at 10 to 5000 μg/ml and absorbance of samples was determined by using ELISA reader.

Conclusions: Based on the results, IS217 (10 μg/ml to 5 mg/ml) demonstrated 46% to 78.7% inhibition of IL-23 secretion as compared to control LPS (1 μg/ml) and IFN-γ (100 ng/ml) levels. Curcumin positive control also led to IL-23 inhibition. Treatment of HaCaT cells with curcumin at 10 μM and resulted in 91.6% and 90.6% inhibition of IL-23 w.r.t. control LPS (1 μg/ml) and IFN-γ 100 ng/ml levels, respectively (FIG. 15).

From the above experiments and results thereof it can be concluded that IS217 is targeting VEGF and/or p38 by downregulating pro-inflammatory IL-23 and IL-17 secretion.

### Example 13: Evaluation of Anti-Inflammatory Potential of IS217 by Inhibition of IL-20 Secretion [50-52]

As IL-20 is highly expressed in psoriatic lesions, in vitro HaCaT based assay was employed to evaluate anti-inflammatory potential of IS217 by IL-20 estimation. Stimulants used for IL-20 secretions in HaCaT cells were IFN-α, CoCl₂, PMA, IL-1-β and UV-B treatment. IL-20 levels were estimated by using ELISA in supernatants. Absorbance of samples was determined by using ELISA reader. No induction of IL-20 was observed. Later THP-1 cell lines were also used for experiment and stimulants were LPS and human IFN-α. Even after incubation period there is no induction of IL-20 secretion in either of the cell lines. Therefore, further experiments to evaluate the anti-IL-20 effects of IS217 were not conducted.

### Example 14: Evaluation of IS217 on Various Tyrosine Kinases Involved in Psoriasis [53-57]

Tyrosine kinases play key role in pathogenesis of psoriasis [1] IS217 was investigated for possible inhibition of various tyrosine kinase, which play crucial role in pathogenesis of psoriasis. The method employed to detect the effect was Z′LYTE activity assay.

Results: IS217 demonstrated inhibition of MAPK1 (MEK 1) by 32%-41% in the concentration range of 10 μg/ml-2000 μg/ml. No inhibition of JAK-STAT pathway was observed by IS217. IS217 exhibited good inhibition of MAPK1 (MEK 1) at concentration range of 10 μg/ml-250 μg/ml. Moderate to low inhibition of other kinases was recorded at low concentration tested of 10 μg/ml.

### Example 15: Evaluation of Effects of IS217 on Topoisomerase II Inhibition [58-61]

Topoisomerase II inhibitory potential of IS217 was evaluated using widely reported kinetoplast DNA as test system in presence of Topoisomerase II enzyme. IS217 was tested at concentrations ranging from 0.01 μg/ml to 1 mg/ml. Doxorubucin hydrochloride and nalidixic acid were used as positive controls at the concentration of 10 μM and 1 mg/ml, respectively.

Inhibition of release of decatenated KDNA products was used as a specific marker to assess the inhibitory activity of IS217 on topoisomerase II enzyme, which was determined by using 1% agarose gel electrophoresis.

Results: IS217 demonstrated no topoisomerase II enzyme inhibition at concentrations ranging from 0.01 μg/ml to 10 μg/ml. But at concentration of 100 μg/ml IS217 demonstrated some extent of inhibition and better inhibition of topoisomerase II was observed at highest tested concentration of 1 mg/ml.

### Conclusions of In-Vitro Studies of Mechanism of Action of IS217:

In pathogenesis of psoriasis, angiogenesis and tyrosine kinases involves cell proliferation, cell migration, DNA Fragmentation and tube formation in endothelial cells. When in vitro cell lines (FBS stimulated proliferation, HaCaT cells and Ea.hy.926 cells respectively) were treated with IS217 led to significant inhibition of cell proliferation and induced DNA fragmentation and showed complete inhibition of endothelial morphogenesis. Generally, in disease VEGF level increase in keratinocytes which leads to angiogenesis, so when treated with IS217, there is a VEGF inhibition in keratinocytes which does not lead to disease progression. RAW264.7 cells (Murine macrophage cell line) which is stimulated with LPS and in HaCaT cells human keratinocytes (HaCaT) TNF-α, IFN-γ, TARC, TSLP, IL-6, IL-8, IL-23, IL-17A and IL-1-0 levels were overexpressed in disease pathogenesis, but when treated with IS217, all the above mentioned cytokines levels are decreased when compared with untreated controls.

The in vitro study results of IS217 with a combination of other marketed drugs (anthralin, methotrexate), IS217 showed a better effect in comparison with alone treatment in combination with methotrexate.

These results demonstrate that IS217 is targeting VEGFR-2 by downregulating p38 kinases and pro-inflammatory IL-23 and IL-17 secretion. Taken together; the experimental study established that it can be possible to treat inflammatory skin diseases such as psoriasis by subcutaneous administration of an antiangiogenic compound.

### Example 16: DMPK Studies

These studies were conducted to study drug metabolism and kinetic profile of IS217 in human circulation system and liver. This gives all parameters like t1/2, clearance rate etc., which are very important in clinical dossier preparation.

16.1: Determination of Binding of Peptide IS217 with Human Plasma Proteins

This study evaluated binding of peptide IS217 with human plasma using ultrafiltration devices (Amicon Filters). Test item at concentration of about 100 μg/ml was incubated with human plasma at 37° C. in water bath for about 1 hour. About 500 μL of test solution was transferred to ultrafiltration devices and centrifuged for about 15 minutes at about 13000 rpm and at 37° C. and analyzed using HPLC method.

The percent protein binding is 53.42%3.20 and free fraction 0.466±0.03. IS217 showed moderate binding affinity to human plasma proteins, high free content demonstrates that therapeutic activity and concentration in plasma is more than the binding. High free content of IS217 in human plasma suggest that low dose can produce higher activity. Hence, high free content was considered as favorable pharmacokinetic property for a drug. The studies concluded IS217 has moderate binding affinity to proteins which shows more efficacy activity.

### 16.2: Determination of In Vitro Stability of Peptide IS217 in Human Plasma and Human Liver Microsomes

This study evaluated metabolic and plasma stability of peptide IS217 in human liver microsomes and human plasma. Metabolic stability assay was performed to determine in vitro half-life and intrinsic clearance of test item. IS217 at a concentration of about 100 μg/ml was incubated with human liver chromosomes in a reaction mixture containing cofactors NADPH regenerating solution A and solution B and 0.1M Phosphate buffer for about 60 minutes at about 37° C. IS217 was monitored during reaction at different time intervals (0, 15, 30, 45 and 60 minutes) and reaction was terminated by addition of cold acetonitrile. Following centrifugation, the supernatant was analyzed on HPLC-UV. Plasma stability assay was performed in human plasma to determine fate of IS217 in plasma. IS217 at a concentration of about 100 μg/ml was incubated with human plasma for about 60 minutes at about 37° C. Samples were removed at (0, 15, 30, 45 and 60 minutes) and quenched with 200 μl of 0.1% TFA in methanol. Samples were processed and supernatant was collected for HPLC-UV analysis to determine plasma stability of IS217.

The results concluded that in metabolic stability assay IS217 was found to be moderately metabolically stable and it was observed that IS217 metabolized ˜58% in 60 minutes. IS217 was found to HLM mediated biotransformation with t1/2 values 47.93 minutes. The apparent intrinsic clearance calculated for IS217 was 14.46 μl/min/mg. In plasma stability assay IS217 was found to be stable in human plasma. It was observed that 90.70%, 105.12%, 85.16% and 91.06% of IS217 was remaining at about 15, 30, 45 and 60 minutes, respectively and the calculated half-life of IS217 in human plasma was found to be 415.5 minutes.

### 16.3: Determination of Inhibition Potential of IS217 for CYP3A4, CYP2C9, CYP2D6, CYP1A2 and CYP2C19 Using High Throughput Inhibitor Screening Kit

Cytochrome P450s are principal enzymes for oxidative metabolism of drugs and other xenobiotics. The clearance of most small molecule drug substances is dependent upon CYP enzymes, their inhibition can lead to overexposure and toxicity. To determine inhibition of cytochrome P450 enzymes by test item the following study was performed.

The study design determined inhibition potential of test item (IS217) for CYP3A4, CYP1A2, CYP2C9, CYP2D6 and CYP2C19 using high throughput inhibitor screening kit. The experiment was performed with concentrations range of 10 μg/mL to 0.005 μg/mL of test compound IS217. Positive controls taken were specific to individual kit, which were taken as follows:

TABLE 4
Positive controls taken to determine inhibition potential
of IS217 for CYP3A4, CYP2C9, CYP2D6, CYP1A2 and CYP2C19

| Enzyme | Fluorescence substrate | Positive control |
|---|---|---|
| CYP1A2 + P450 | CEC (3-Cyno-7- | Furafylline |
| | Ethoxycoumarin) | |
| CYP3A4 + P450 | BFC (7-BEnzyloxy- | KTZ |
| | trifluoromethyicoumarin) | (Ketoconazole) |
| CYP2C9 + P450 | MFC (7-Methoxy-4- | SFZ |
| | trifluoromethyicoumarin | (Sulfaphenazole) |
| CYP2C19 + P450 | CEC (3-Cyno-7- | TCP |
| | Ethoxycoumarin) | (Tranylcypromine) |
| CYP2D6 + P450 | AMMC (3-[2-(N,N-diethyl- | Quinidine |
| | N-methylamino) ethyl]-7- | |
| | methoxy-4-methylcoumarin) | |

TABLE 5
Test item (IS217) was incubated with cofactors NADPH,
enzyme (CYP3A4, CYP2C9, CYP2D6, CYP1A2 and CYP2C19)
and respective fluorescent substrate at 370° C.

| CYP1A2 | CYP3A4 | CYP2C9 | CYP2C19 | CYP2D6 |
|---|---|---|---|---|
| 15 minutes | 30 minutes | 45 minutes | 30 minutes | 30 minutes |

At the end of incubation period, metabolism of fluorescent substrates to their metabolites was read using fluorescent multiwall spectrophotometer (Biotek reader). Very less (<20% at 10 μg/mL) to no inhibition potential was observed towards enzymes CYP3A4, CYP2C9, CYP2D6, CYP1A2 and CYP2C19. IS217 showed slightly higher potential towards CYP1A2 as compared to others.

### Example 17: In Vitro Mutagenicity Safety Studies

17.1: Bacterial Reverse Mutation Test in Salmonella typhimurium Tester Strains:

This study was conducted to test whether IS217 shows any mutagenicity i.e. induce any mutations in cells.

IS217 is non mutagenic as it did not induce any gene mutations at histidine locus by base pair changes or frame shift in the presence and absence of metabolic activation in Salmonella typhimurium five tester strains upto 1000 μg/plate concentration. All the in vitro studies showed good results. This was confirmed by in vivo studies which are mandatory for any drug, as a part of safety toxicological studies.

### 17.2: In Vitro Mammalian Chromosomal Aberration Assay of IS217 in Human Peripheral Blood Lymphocytes

This study was conducted to test whether IS217 shows any genotoxicity i.e., induce any mutations in cells.

Test item did not induce chromosomal aberration upto 1000 μg/ml concentration of culture medium both in presence and absence of human peripheral blood lymphocytes under experimental conditions.

### 17.3: In Vitro Hemolytic Potential of IS217 in Isolated Rabbit Erythrocyte

This study was conducted to test whether IS217 induced haemolysis or not. The study was conducted in IV intended concentration.

The mean haemolytic index of IS217 is 0% which meant non-haemolytic. The mean hemoglobin value of IS217 was found to be non-significant when compared with negative control and positive control.

### Example 18: In Vitro Combinational Studies with Anthralin and Methotrexate

18.1: In-Vitro Assessment of Cytotoxicity of Methotrexate in Combination with IS217 by Combination Index Method Using Human Keratinocytes

Assessment of cytotoxicity of methotrexate in combination with IS217 on proliferation of human keratinocytes (HaCaT) cells was performed by using combination index method. HaCaT cell line is maintained by using DMEM, 10% FBS, CO₂ incubator and after overnight incubation cells were replenished by using about 1% FBS and then the cells were treated with different concentrations (0.01, 0.1, 0.25, 0.5, 1, 5 μM) of methotrexate alone, IS217 (10, 50, 500, 1000, 2000, 5000 μg/ml) alone, combination of methotrexate (0.01, 0.1, 0.25, 0.5, 1, 5 μM) and IS217 (10, 50, 500, 1000, 2000, 5000 μg/ml) to achieve different combinations.

IS217 was added to the above combinations every 24 hours (i.e., three doses) whereas methotrexate was added only once in 72 hours during the 72 hours of incubation. Then the plates were subjected for MTT assay and percentage inhibition (cytotoxicity) corresponding to each treatment was calculated.

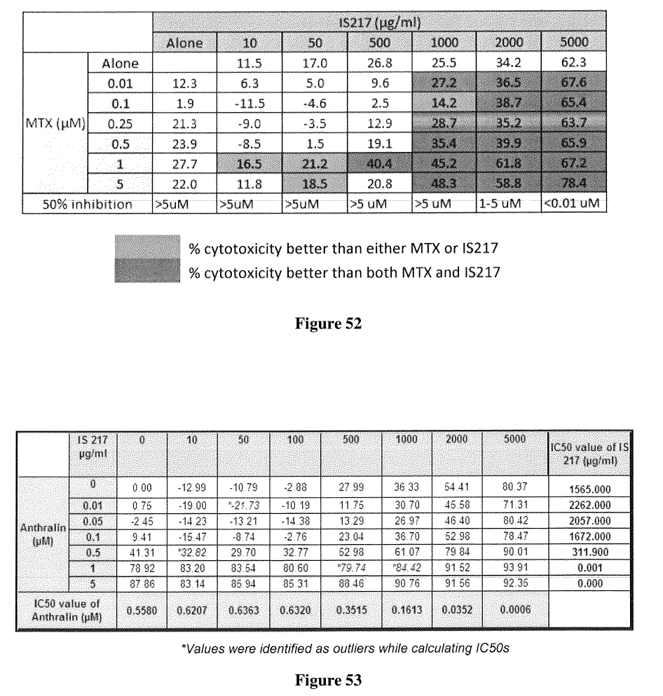

Results and conclusions: When methotrexate was tested alone, 50% inhibition of cell proliferation (IC50) was not achieved and in combination with IS217 in concentration range of 10-1000 μg/ml, IC50 was still not achieved (FIG. 52). However, at 2000 μg/ml of IS217, >50% inhibition of cell proliferation was observed between 1 and 5 Mm of methotrexate. Finally, at 5000 μg/ml of IS217, 50% inhibition was further lowered to <0.01 μM of methotrexate. The above results concluded that treatment with combination of IS217 and methotrexate showed synergistic effects.

18.2: In Vitro Assessment of Effect of Combination of Anthralin with IS217 by Combination Index Method Using Human Keratinocytes

The study aimed to determine the combination of anthralin with IS217 on proliferation of keratinocytes by combination index method. Here MTT assay, BioTek Synergy HT multimode readers were used for determination of cell viability by calculating CI (Combination Index) method based on IC50 values (concentration leading to 50% inhibition of cell growth).

For assessing, human epidermal keratinocytes (HaCaT) were treated with test item IS217 at concentration range from 10-5000 μg/ml. Anthralin at about 0.01 to 10 μM, and combination of anthralin with IS217 concentrations with about 72 hours of incubation, and retreatment done with IS217 every 24 hours (i.e., 3 doses) and anthralin only once in 72 hours. After the incubation, the plates were taken out and MTT was added to wells followed by additional incubation for about 3 hours at 37° C. About 150 μl of DMSO was added to aspirated supernatant and observed at 540 nm using BioTek Synergy HT plate reader. IC50 values of the above items were obtained using Graph pad prism version 4.01.

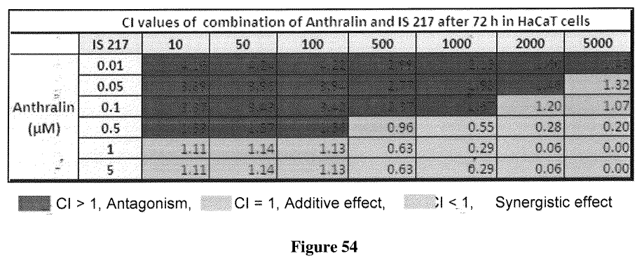

Results: The combination of anthralin and IS217 exerted good synergetic effects at doses ranging from 500 to 5000 μg/ml of IS217 and 0.5 to 5 μM of anthralin (FIG. 53 and FIG. 54). However, at concentration of 500 μg/ml of IS217 and 1 μM of anthralin, additive effect was observed.

Conclusions: Based on the results, it was concluded additive effect of IS217 when used in combination with anthralin and methotrexate by using human keratinocytes cell lines. IS217 enhanced the inhibitory cytotoxic potential of MTX. IS217 showed good results with a combination of anthralin and methotrexate.

### Example 19: Development of Pre-Formulation and Determination of Maximum Feasible Concentration for Peptide IS217

This study is important in CMC preparation of any drug. The study was conducted to develop suitable pre-formulations of IS217 for four administration routes selected for in vivo studies and to determine maximum concentration of IS217 peptide which is feasible in these pre-formulation i.e., maximum feasible concentration (MFC). Four different aqueous based pre-formulations of IS217 like 10% v/v Tween 20, 20% v/v glycerol, 10% PG v/v in 0.9% NaCl solution and 0.9% NaCl alone were prepared and were first subjected to stability study at intended use of temperature (RT) for about 24 hours. HPLC based assay method was used to evaluate the stability. During the period of 24 hours pre-formulations showed no visual changes in physical appearance from initial sample.

Out of these, four aqueous based pre-formulation, 0.9% NaCl solution was selected for intravenous (IV) and intraperitoneal (IP) route and also the aqueous vehicle for co-solvent pre-formulation, based on its stability and suitability at physiological conditions. For subcutaneous (SC)/intramuscular route (IM), a co-solvent-based pre-formulation was developed. 10% propylene glycol (PG) in 0.9% NaCl solution was selected as pre-formulation for SC/IM. Maximum feasible concentration of IS217 in both the pre-formulations was greater than 50 mg/ml.

### Example 20: Validation of Liquid Chromatography with Tandem Mass Spectrometry (LC-MS/MS) Method for Determination of Peptide IS217 in Rat Plasma

This study was conducted to validate a LC-MS/MS based bioanalytical method for estimation of peptide IS217 in rat plasma. Quattro micro micromass LC-MS/MS, Waters 2695 HPLC separation module, MassLynx version 4.0 software (Micro mass, UK) were the instruments used and rat plasma containing EDTA as an anticoagulant was used as biological matrix. Bupivacaine hydrochloride was used as internal standard.

Based on above results it was concluded that a LC-MS/MS based method for estimation of peptide IS217 in rat plasma was developed and validated for calibration curve range from 5.18 ng/mL to 384 ng/mL. The method was found to be precise, linear, accurate, selective and stability indicating.

### Example 21: In Vivo Animal Studies: Efficacy Studies of IS217

Series of studies with IS217 in animal models of psoriasis was conducted as summarized below. (Vehicle: 10% v/v propylene glycol in 0.9% NaCl solution)

TABLE 6
Summary of pre-clinical studies - pharmacology

| | | | | |
|---|---|---|---|---|
| | | Route of | | Noteworthy |
| Species/Strain | Study design | administration | Dose/s | findings |

Study type: pharmacology appropriate route of administration

| | | | | |
|---|---|---|---|---|
| IMQ induced | IS217 at various | IS 217 via IM, | IS 217: SC | 1.2 mg/kg SC |
| psoriasis | concentrations in | IV, IP and SC | (0.3, 0.6 and1.2 | dose lowered |
| model Female | different route of | route of | mg/kg | TNF-α and IL-23 |
| Balb/C mice | administrations | administrations | IS 217: IM | levels, rete peg |
| (9-12 weeks) | 10 groups with 6 | | 1.2 mg/kg | proliferation in |
| | animals in each | | IS 217: IV | histopathology |
| | group (Total 60) | | 1.2 mg/kg | examination of |
| | | | IS 217: IP | ear |
| | | | 1.2 mg/kg | (p < 0.01) |
| | | | (q.d. for 8 days) | 0.3 mg/kg SC |
| | | | | reduced |
| | | | | hyperkeratosis, |
| | | | | edema, |
| | | | | inflammatory |
| | | | | cell infiltration |
| | | | | and ear thickness |
| | | | | in histopathology |
| | | | | examination of |
| | | | | ear(p < 0.01) |

Study type: pharmacology

| | | | | |
|---|---|---|---|---|
| Imiquimod | IS217 at various | IS217 and | IS 217: SC | Significant ear |
| (IMQ) induced | concentrations in | MTX both | 4.8, 2.4, 1.2, | thickness |
| psoriasis | comparison with | administered | 0.6, 0.3, 0.15 | reduction, |
| model | MTX | SC'ly | mg/kg q.d. | lowering of |
| Female Balb/C | 9 groups with 8 | | MTX: SC | psoriatic score |
| mice (8-10 | animals in each | | 1.5 mg/kg; q2d | PASI and ear |
| weeks) | group (total 72) | | X 4 | punch biopsy |
| | | | (8 days) | Histopathological |
| | | | | findings show |
| | | | | higher % |
| | | | | inhibition of ear |
| | | | | inflammation and |
| | | | | edema in |
| | | | | IS217(1.2 mg/kg) |
| | | | | Ear homogenate − |
| | | | | TNF-α, IL-23, |
| | | | | MPO reduction |
| | | | | by IS217 |
| | | | | (p < 0.01) |

Study type: pharmacology efficacy in combination with MTX and Betamethasone

| | | | | |
|---|---|---|---|---|
| TPA induced | IS217 alone and in | Subcutaneously | IS217: SC | Decreased ear |
| ear | combination with | (SC) for IS217 | 1.2 mg/kg; q.d. | thickness |
| inflammation | MTX and | and MTX | MTX: SC | (absolute, |
| model of | Betamethasone. | Topical for | 1.5 mg/kg; q2d | histopathology, |
| psoriasis | 11 groups with 8 | Betamethasone | Betamethasone: | ear punch |
| Male C57B1/6 | animals in each | | Topical | biopsy) |
| mice (6-8 | group (Total 88) | | 0.01% and | IS217 (1.2 mg/kg) < |
| weeks) | | | 0.001% | MTX |
| | | | (10 days) | (1.5 mg/kg) |
| | | | | (p < 0.01) |
| | | | | IS217 |
| | | | | combination with |
| | | | | MTX and |
| | | | | Betamethasone |
| | | | | (p < 0.001) |
| | | | | IS217 decreased |
| | | | | inflammatory |
| | | | | cytokines in ear |
| | | | | homogenate |
| | | | | (ELISA and |
| | | | | histopathology |
| | | | | score) IL-23, IL- |
| | | | | 6, TNF-α and |
| | | | | MPO. |
| | | | | IS217 decreased |
| | | | | edema and |
| | | | | hyperkeratosis |

Study type: pharmacology

| | | | | |
|---|---|---|---|---|
| Male C57B1/6 | IS217 at various | IS217 and | IS217: SC | Decreased ear |
| mice (6-8 | concentrations in | MTX both | 4.8, 2.4, 1.2, | thickness |
| weeks) | comparison with | administered | 0.6, 0.3, 0.15 | (absolute, |
| TPA induced | MTX | SC | mg/kg q.d. | histopathology, |
| ear | 9 groups with 8 | | MTX: SC | ear punch |
| inflammation | animals in each | | 1.5 mg/kg; q2d | biopsy) |
| model of | group (total 72) | | X 5 (10 days) | IS217 (all doses) < |
| psoriasis | | | | MTX |
| | | | | (p < 0.001) |
| | | | | Histopathological |
| | | | | findings show |
| | | | | higher % |
| | | | | inhibition of ear |
| | | | | inflammation, |
| | | | | hyperkeratosis |
| | | | | and edema in IS |
| | | | | 217(1.2 mg/kg) |
| | | | | against MTX |
| | | | | Ear homogenate |
| | | | | ELISA − TNF-α, |
| | | | | IL-23, IL-6, |
| | | | | MPO reduction |
| | | | | by IS217 |
| | | | | (p < 0.01) |

### Example 22: Evaluation of Anti-Psoriatic Potential of IS217 in Imiquimod (IMQ) Induced Psoriasis Model Using Balb/c Mice

Generally, in psoriasis, the pro inflammatory cytokines like IL-17, IL-23, TNF and VEGF levels increase. This is studied on IMQ and TPA induced psoriasis model which developed skin pathology highly correlated to human psoriasis and is widely accepted as reliable preclinical psoriasis model.

Topical application of IMQ, a TLR7/8 ligand and potent immune activator, induced and exacerbated psoriasis, a chronic inflammatory skin disorder. Daily application of IMQ on mouse back skin induced inflamed scaly skin lesions resembling plaque type psoriasis.

These lesions showed increase in epidermal proliferation, abnormal differentiation, epidermal accumulation of neutrophils in micro abcesses, neoangiogenesis, and infiltrates consisting of CD4 (+) T cells, CD11c (+) dendritic cells, and plasmacytoid dendritic cells. IMQ induces epidermal expressions of IL-23, IL-17A and IL-17F, as well as an increase in Th17 cells in spleen. IMQ-induced dermatitis is partially dependent on presence of T cells.

The sole application of innate TLR7/8 ligand IMQ rapidly induced a dermatitis closely resembling human psoriasis. This is a rapid convenient model that allows further elucidation of pathogenic mechanisms and evaluation of new therapies in psoriasis.

### 22.1: Experimental Procedure

The study was conducted in healthy, adult female Balb/c mice. All the animals were acclimatized to laboratory condition prior to experiment initiation.

Animals were randomized into 10 groups containing 6 animals each group, based on their body weight. Approximately 63 mg of IMQ cream (5%) (containing 3.125 mg of IMQ) was weighed and applied topically each on dorsal area (shaved back) and right ear of mice for 8 days in group 3-10. Groups 1 and 2 were served as controls and were treated with appropriate vehicle. Later groups 1 and 2 were merged for better calculation purpose.

TABLE 7
IS217 was administered through various routes of administration
daily for 8 days as mentioned below. Allocation of animals
and mode of administration is as follows:

| | | | | | No of |
|---|---|---|---|---|---|
| Groups | Treatment | volume | | Route | animals |
| G1 | Normal control | 50 | μL | IM | 6 |
| G2 | Normal control | 4 | mL/kg | IV | 6 |
| G3 | Disease control | 50 | μL | IM | 6 |
| G4 | Disease control | 4 | mL/kg | IV | 6 |
| G5 | IS217 | 1.2 | mg/kg | Subcutaneous | 6 |
| G6 | IS217 | 0.6 | mg/kg | Subcutaneous | 6 |
| G7 | IS217 | 0.3 | mg/kg | Subcutaneous | 6 |
| G8 | IS217 | 1.2 | mg/kg | IM | 6 |
| G9 | IS217 | 1.2 | mg/kg | IV | 6 |
| G10 | IS217 | 1.2 | mg/kg | Intra peritoneal | 6 |

Body weight, ear thickness and dorsal skin thickness was measured daily using digital caliper. Erythema index was recorded by mexameter and clinical scoring was also done daily for dorsal psoriatic area and clinical scoring was also done daily for dorsal psoriatic area.

On day 9, animals were anesthetized for blood collection followed by euthanasia by C02 asphyxiation as per in-house approved standard protocol.

About 4 mm ear punch biopsy was collected, weighed and fixed in about 10% neutral buffer formalin for histopathological analysis (via H and E staining). Remaining ear was excised and stored at about −80° C. for inflammatory cytokine analysis. Dorsal skin was also collected, and half was kept in formalin. Spleen was also collected and weighed and kept in formalin.

In this study three different routes i.e., SC, IM and IV at different doses ranging from 0.3, 0.6 and 1.2 mg/kg were used to see dose response curve, the results were as follows:

### Results: Observation/Calculations

No significant change was observed in body weight and ear thickness except on disease control.

There was a trend in absolute ear thickness reduction on IS217 SC, IV and IM treatment. IS217 treatment resulted in absolute ear thickness reduction across the groups with maximum inhibition of 80.36% @1.2 mg/kg dose and similar trend was observed in ear punch biopsy weight.

### 22.1.1: Percentage Inhibition of Ear Inflammation:

Percentage inhibition of ear inflammation was calculated from day 6 of disease induction was found to be maximum. No dose dependent activity was found in IS217 treatment groups and low dose showed maximum inhibitory response of 32.08% and by IM route. IS217 showed 59.12% of maximum inhibitory activity.

### 22.1.2: Ear Biopsy Weight:

On day 9, all the experimental animals were euthanized, and a standard 4 mm ear punch biopsy was collected and weighed. Significant increase in biopsy weight was observed in disease control (p<0.01) when compared to normal control.

Among all IS217 treated groups; IS217 (0.3 mg/kg) SC treatment group showed maximum effect in comparison to other tested doses as well as route of administration.

### 22.1.3: Erythema Index

Erythema index was measured daily for 9 consecutive days using mexameter. Readings for each animal were recorded at three places and their average value was considered for further calculations.

Significant increase (p<0.001) in erythema index was observed in disease control groups when compared with normal control from day 2 to day 5 and on day 8 (P<0.05).

No significant reduction in erythema index was observed in any treatment groups.

### 22.1.4: Psoriatic Scoring of Dorsal Area—Erythema, Scaling and Thickening

Psoriatic scoring was done for 9 consecutive days in all experimental groups. Erythema, scaling and thickening were scored independently on a scale of 0 to 4: 0, none; 1, slight; 2, moderate; 3, marked; 4, very marked. The cumulative score (erythema plus scaling plus thickening) served as a measure of the severity of inflammation (scale 0-12). Significant increase (P<0.01; P<0.001) in cumulative psoriatic score was observed in disease control groups when compared with normal control from day 3 to day 9.

No significant reduction was observed in any of the treatment groups

### 22.1.5: Spleen Weight

Mean spleen weight was calculated and represented in tabulated and graphical form.

Significant increase in spleen weight was observed in disease control (p<0.01) when compared to normal. No effect on spleen weight reduction was found in IS217, SC treatment groups and maximum reduction was observed in IV treatment group.

None of the treatment groups were found to be statistically significant when compared with disease control.

### 22.1.6: Analysis of Inflammatory Cytokines

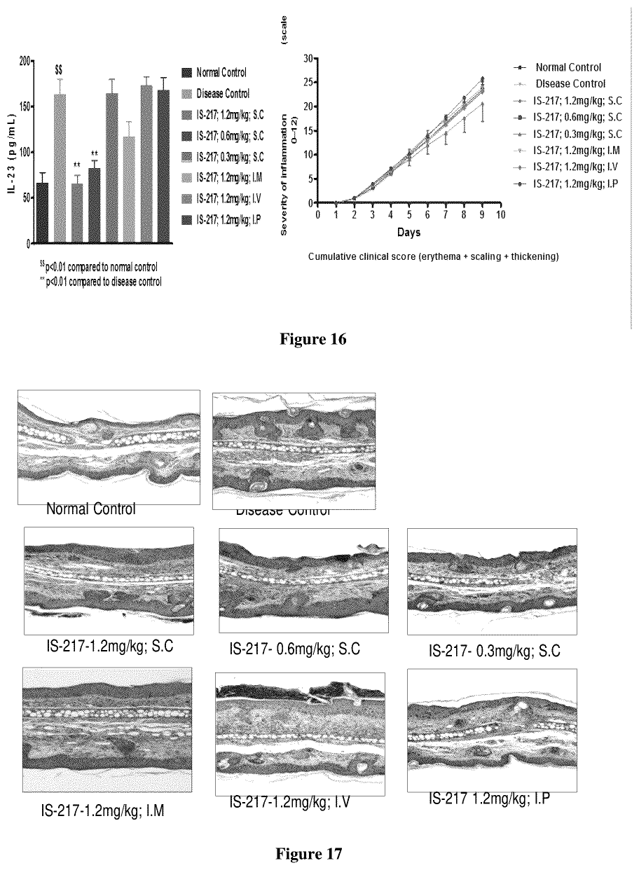

TNF-α, IL-17 and IL-23 were analyzed in ear tissue homogenate samples by ELISA. Significant increase in all three tested inflammatory cytokines were observed in disease control (p<0.01) when compared to normal control.
TNF-α level was found to be partially reduced on 1.2 mg/kg SC treatment with IS217, among all the routes and doses tested maximum effect was seen at this dose.
In IL-17 analysis, maximum inhibition was obtained on IV treatment
In IL-23 analysis, clear dose dependent suppression of IL-23 levels was observed on SC treatment with IS217 and with 1.2 mg/kg and 0.6 mg/kg treated groups.
Animals treated with IS217 @dose of 1.2 and 0.6 mg/kg showed 59.85% and 49.68% reduction in IL-23, the key cytokine in psoriasis pathogenesis and TNF-α was also reduced to 24.25% and 13.94%, respectively when compared to the controls (FIG. 16).
However, no trend was observed in IL-17A and spleen weight.

### 22.1.7: Histopathological Findings

Hematoxylin and eosin dye-stained ear histopathogical photographs were subjected for epidermal ear thickness measurement using UTHSCSA image tool; version 3.0.SC administration of IS217 at low dose of 0.3 mg/kg resulted maximum reduction of IMQ induced hyperkeratosis, edema, Rete Peg proliferation, inflammatory cell inflammatory cell infiltration (FIG. 17).

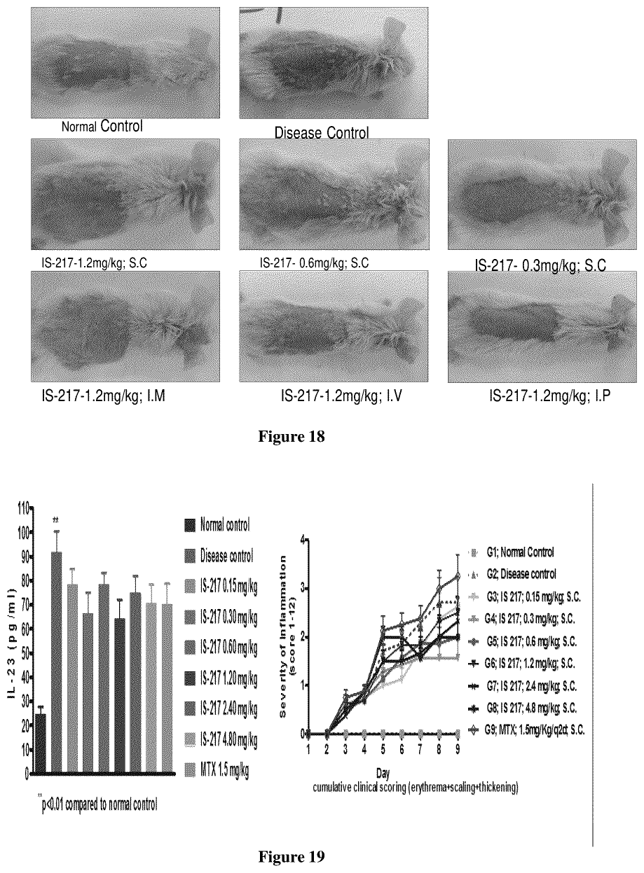

Conclusions: Based upon the findings, it can be concluded that IS217 SC route exerted potential anti-psoriatic activity perhaps through modulation of IL-23 pathway and suppression of cytokine production (FIG. 18).

### 22.2: Dose Response Studies of IS217 in IMQ Induced Psoriasis Model Using Balb/c Mice

The experimental procedure and doses and route of administration are same as IMQ model. Test item IS217 was administered subcutaneously to diseased animals at different dose levels of 0.15 mg/kg, 0.30 mg/kg, 0.60 mg/kg, 1.2 mg/kg, 2.4 mg/kg and 4.8 mg/kg.

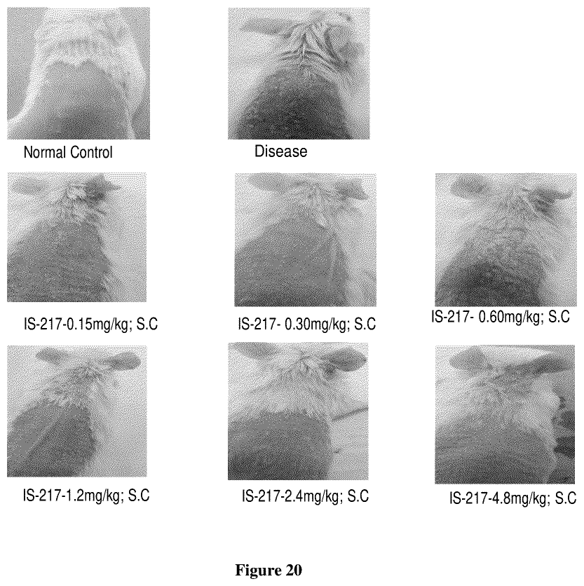

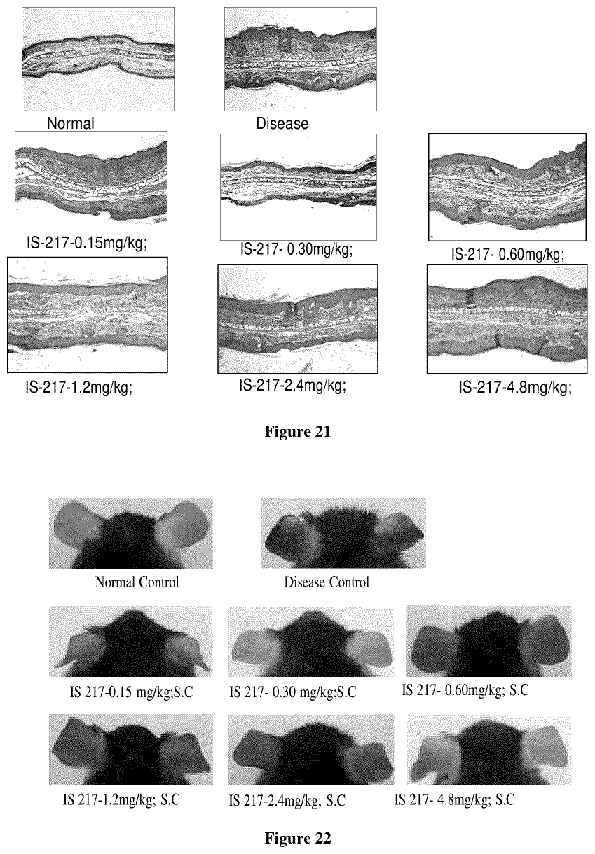

Results: There was a trend in animals treated with IS217 through SC in ear thickness (life phase), punch biopsy, cumulative psoriatic scoring and spleen weight in all the tested doses; however, dose level of 0.3 mg/kg showed maximum effect. IS217 at the dose of 0.3 mg/kg exhibited 36.34% inhibition of ear thickness. Maximum inhibition of 29.90% was obtained at the dose of 1.20 mg/kg followed by 0.30 mg/kg where 27.75% inhibition was found. However, no trend was observed in TNF-α level (FIG. 19 to FIG. 21).

### 22.3: Evaluation of Dose Response of IS217 in 12-O-Tetradecanoylphorbol-13-Acetate (TPA) Induced Ear Inflammation Model in C57BL/6 Mice

The experimental procedure and doses and route of administration are same as IMQ model.

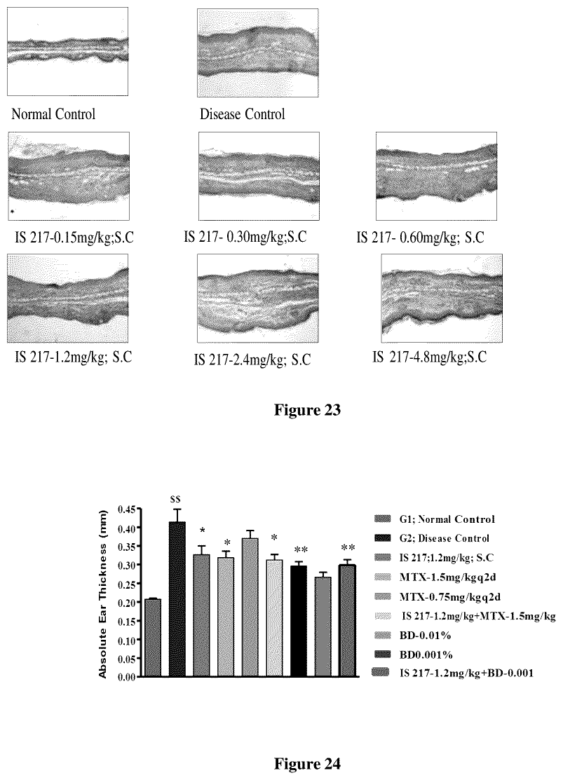

Results: Treatment with IS217 resulted in significant reduction in absolute ear thickness at the doses from 1.2 mg/kg-4.8 mg/kg with maximum ear thickness inhibition of 80.36% at dose of 1.2 mg/kg. Similar trend was observed in ear punch biopsy weight and histopathological scoring. Animals treated with 4.8 and 2.4 mg/kg showed 46.77 and 30.51% inhibition of TNF-α level, respectively. In case of IL-6, maximum inhibition of 55.27% was observed at the dose of 2.4 mg/kg of IS217 and the animals treated with IS217 at 0.60 mg/kg showed 42.12% reduction in IL-23 levels in ear tissue homogenate (FIG. 22 and FIG. 23).

22.4: Anti-Psoriatic Potential of IS217 Alone and in Combination with Methotrexate and Betamethasone in 12-O-Tetradecanoylphorbol-13-Acetate (TPA) Induced Ear Inflammation Model in C57bl/6 Mice

The experimental procedure, doses and route of administration were same as IMQ model. Test item IS217 was administered subcutaneously either alone or in combination with standard methotrexate (MTX) and betamethasone (BD) to diseased animals.

Results: Treatment with IS217 resulted in significant reduction in absolute ear thickness both alone as well as in combination with standard drugs. IS217 in combination with low dose of MTX (0.75 mg/kg) exhibited better effect in comparison with alone treatment. IS217, MTX (1.5 mg/kg), MTX (0.75 mg/kg) and IS217+MTX (0.75 mg/kg) exhibited 44.77%, 46.06%, 24.95% and 52.48% inhibitory activity against TPA induced increase in ear thickness, suggesting partial synergistic activity. Similar trend was observed in punch biopsy weight. By all above observations, IS217 at 0.3-1.2 mg/kg SC showed prominent dose dependent results in reduction of IL-23 levels which is key biomarkers in pathogenesis in psoriasis (FIG. 24).

Conclusions of in vivo studies of IS217: Dose response studies done in IMQD and TPA model—in Balb/c and C57BL/6 mice, respectively, demonstrated that 1.2 mg/kg dose has potential effect on psoriasis. In vivo efficacy: TPA model-combination study with MTX and BD: IS217 demonstrated good effect in combination with MTX, but not with BD. Thus, IS217 can be used as adjuvant therapy also. In all the above observations, IS217 at 0.3 to 1.2 mg/kg SC showed prominent dose dependent resulted in reduction of IL-23 levels which is key biomarkers in pathogenesis in psoriasis. IS217 at 1.2 mg/kg SC showed a significant reduction in MPO levels also.

### 22.5: Proof of Concept Studies

From the overall above in vitro and in vivo studies, it could be concluded that IS217 used in their respective doses, exhibit remarkable potential in efficaciously attenuating the symptoms and processes underlying psoriasis-like symptoms in mice. Since the IS217 being a lytic synthetic peptide having anti-angiolytic activity and anti-inflammatory properties could antagonize the deleterious effects of the pro inflammatory mediators responsible for the onset of the chronic disease, they may therefore prove to have potential in preventing, delaying, and curing the disease and can open the way to new therapeutic strategies for psoriasis treatment.

However, though IMQ-induced psoriasis in mice may share the cytokine profile and histopathological findings to those of the psoriatic lesions in humans, further research is required to validate that they are the corresponding disease expressions. The current findings on the psoriasis-like mice model could be found appropriate on the human psoriatic model only after such validations and further clinical research. Furthermore, there is still a room for further research in future for elucidation of the elaborate mechanisms followed by IS217 in regulating the internal factors for effectively dealing with the disease.

Taken together; the studies of present invention provide proof of concept that it might be possible to treat inflammatory skin diseases such as psoriasis by subcutaneous and topical administration of an antiangiogenic compound.

### 22.6: Proof of Concept (PoC) Efficacy Study for SC Route and Topical Route in IMQ-Induced Psoriasis Mice Model

The study objective was to validate the efficacy of IS 217 through Parental route and to study the efficacy of 1% IS 217 Cream Formulation through topically and to fix dose levels and treatment mode for further proof of concept studies and included with SC dose (06 and 1.2 mg/kg) as proof of concept studies.

The study was conducted to evaluate efficacy of test compound IS217 (topical and subcutaneous route) on IMQ induced psoriasis mouse model.

Test items: IS217 cream formulation (topical treatment) and IS217 powder vials (subcutaneous treatment) along with placebo were provided.

Standard Drugs: 1.0.01% Batamethasone cream (Betnovate®); 100 mg/4 ml bevacuzimab (Avastin®)

Study animals: 8-9 weeks old male BALB/c mice were used for the study.

Treatment period was from day 1 to day 14. IMQ cream application: 5% IMQ cream, 65 mg was applied on shaved back and 5 mg on each ear during study period. Treatment regimen: treatment to be given twice a day-test drug high dose. 2-hour difference was taken between application of IMQ and application of test drug. 8-hour difference was taken between second application of test drug.

TABLE 8
Treatment schedule and grouping of animals was as follows

| Sr. No. | Treatment Groups | n |
|---|---|---|
| 1 | Native | |
| 2 | Vehicle (cream base) + IMQ cream | 12 |
| 3 | 1% IS217 cream (topical, 50 mg on back and 5 mg | 12 |
| | on each ear total area about 20-25 cm²) + | |
| | IMQ cream | |
| 4 | Post 03 days of IMQ application, treatment with 1% | 12 |
| | IS217 (topical 50 mg on back and 5 mg on each ear | |
| | total area about 20-25 cm²) for 14 days | |
| 5 | Post 07 days of IMQ application, treatment with 1% | 12 |
| | IS217 (topical 50 mg on back and 5 mg on each ear | |
| | total area about 20-25 cm²) for 14 days | |
| 6 | Standard drug treatment- Betamethasone, topical 50 mg | 12 |
| | on back and 5 mg on each ear | |
| 7 | IS217, 0.6 mg/kg, SC, QD (daily, once) | 12 |
| 8 | IS217, 1.2 mg/kg, SC, QD (daily, once) | 12 |
| 9 | Bevacuzimab (Avastin ®), 10 mg/kg, SC, twice a week | 12 |
| 10 | Vehicle (subcutaneous QD group) + IMQ cream | 06 |

### Test Compound Formulation Preparation

The dose formulations were prepared immediately before administered to respective group animals:

### Test Compound Preparation:

Topical formulation 1% IS217 were used by weighing required quantity

### Test Compound IS217 Preparation SC Route:

Required quantity of IS217 compounds was weighed on Sartorius weighing balance. Weighed amount was dissolved in 10% Propylene glycol.

All compounds were prepared every day fresh before dosing.

### Preparation of 10% Propylene Glycol:

2 ml of propylene glycol was mixed in 18 ml of 0.9% saline.

### Standard Compound Preparation: Betamethason Cream (Commercial Formulation Betnovate®)

Required quantity of Betamethasone cream was weighed every day for the application. Bevacizumab (commercial formulation Avastin®): 100 mg/4 ml Avastin formulation was diluted with saline to make 10 mg/5 ml.

TABLE 9
Treatment groups and dosing schedule:

| | Treatment | Route of | | | |
|---|---|---|---|---|---|
| Group | Group | administration | Dose | Frequency | n |
| 1 | Normal control | Topical | 50 mg (back), | BID | 12 |
| | | | 5 mg each ear | | |
| 2 | Vehicle | Topical | 50 mg (back), | BID | 12 |
| | | | 5 mg each ear | | |
| 3 | 1% IS217 cream | Topical | 50 mg (back), | BID | 12 |
| | | | 5 mg each ear | | |
| 4 | 1% IS217 creams | Topical | 50 mg (back), | BID | 12 |
| | (Treatment | | 5 mg each ear | | |
| | initiated Post | | | | |
| | 03 days of IMQ | | | | |
| | application) | | | | |
| 5 | 1% IS217 creams | Topical | 50 mg (back), | BID | 12 |
| | (Treatment | | 5 mg each ear | | |
| | initiated post | | | | |
| | 07 days of IMQ | | | | |
| | application) | | | | |
| 6 | Betamethasone | Topical | 50 mg (back), | QD | 12 |
| | cream | | 5 mg each ear | | |
| 7 | IS217 | SC | 0.6 mg/kg | QD | 12 |
| 8 | IS217 | SC | 1.2 mg/kg | QD | 12 |
| 9 | Bevacuzimab | SC | 10 mg/kg | Twice | 12 |
| | | | | week | |
| 10 | Vehicle IS217 | SC | | QD | 06 |

### Topical Application of IMQ to Induce Psoriasis Like Symptoms:

IMQ (Imigod, Glenmark pharmaceuticals) was weighed every day prior to application.
65 mg of 5% IMQ cream was applied on shaved back of animals with help of spatula. 5 mg IMQ cream was applied on each ear of mouse.
IMQ application was done every day for 14 days and on day 15 studies were terminated. (Except for group 4 and group 5)
Group 4 was terminated on day 17 (post 14 days of treatment)
Group 5 was terminated on day 21 (post 14 days of treatment)
Group 4 and 5 received similar duration of test formulation as per group 3.
Group 4 received 17 days IMQ application, group 5 received 20 days IMQ application.

### Application of Reference Standard and Test Compound:

Treatment to all the groups were done two hours post IMQ cream application
Formulation for topical application were weighted daily on Sartorius balance
Formulation for SC treatment was prepared fresh every day as per procedure mentioned above.
Standard drug Betamethasone was weighed daily and applied topically to respective treatment group
Bevacizumab was prepared fresh at the time of dosing.
All the treatments were given as per study design

TABLE 10
Parameters monitored during study period

| Sr. No. | Parameter | Frequency | Details/Specification |
|---|---|---|---|
| 1 | Ear Thickness | Twice a | Using Digital caliper, Mitutoyo micrometer. |
| | | week | |
| 2 | Erythema | weekly | Quantitative observation by observer blind |
| | Score | once | to the treatment group. |
| 3 | Scaling Score | weekly | Quantitative observation by observer blind |
| | | once | to the treatment group. |
| 4 | Dorsal skin | Terminal | Upper, Middle and lower side of the back |
| | thickness | | and final value recorded as Average of |
| | | | these three recordings |
| 5 | Ear biopsy | Terminal | Weight of both ears |
| | weight | | |
| 6 | Spleen weight | Terminal | Weight of Spleen |
| 7 | Body weight | Twice a | The % change in body weight for each |
| | | week | animal was calculated |
| 8 | Inflammatory | Terminal | Representative samples, n = 6 from each |
| | Cytokines: | | group for each cytokine by ELISA. |
| | Skin | | IL-17, IL-6, TNF-α, VEGFA, IL-23, IL-4, |
| | Homogenate | | IL-13 |
| 9 | Inflammatory | Terminal | Representative samples, n = 6 from each |
| | Cytokines: Ear | | group for each cytokine by ELISA. |
| | Homogenate | | IL-17, IL-6, TNF-α, VEGFA, IL-23, IL-4, |
| | | | IL-13 |
| 10 | Inflammatory | Terminal | Representative samples, n = 6 from each |
| | Cytokines: | | group for each cytokine by ELISA. |
| | Serum | | IL-17, IL-6, TNF-α, VEGFA, IL-23, IL-4, |
| | | | IL-13 |
| 11 | Histopathology | Terminal | n = 6 samples from all groups were analyzed |
| | (Skin) | | for Histopathology |
| 12 | Histopathology | Terminal | n = 6 samples from all groups were analysed |
| | (Ear) | | for Histopathology |
| 13 | Photographs | Twice a | Representative (n = 4) photographs of mice |
| | | week | skin on Day 1, 4, 8, 11, 14, 17 and 21 |

### Results

Upon topical application of IMQ cream, it showed increase in inflammation, which is evident by significant increase in ear thickness in IMQ application group (vehicle group) compared to naïve control. Application of IMQ increases production of pro-inflammatory cytokines and chemokines. It is evident that IL-17/IL-23 and Th1 pathway are primarily involved in development of psoriasis.

### General Observations:

In all treatment modes, i.e., prophylactic, semi therapeutic and therapeutic modes, there was no significant change in body weight was observed in 1% IS217 treatment group compared to vehicle during the study period.

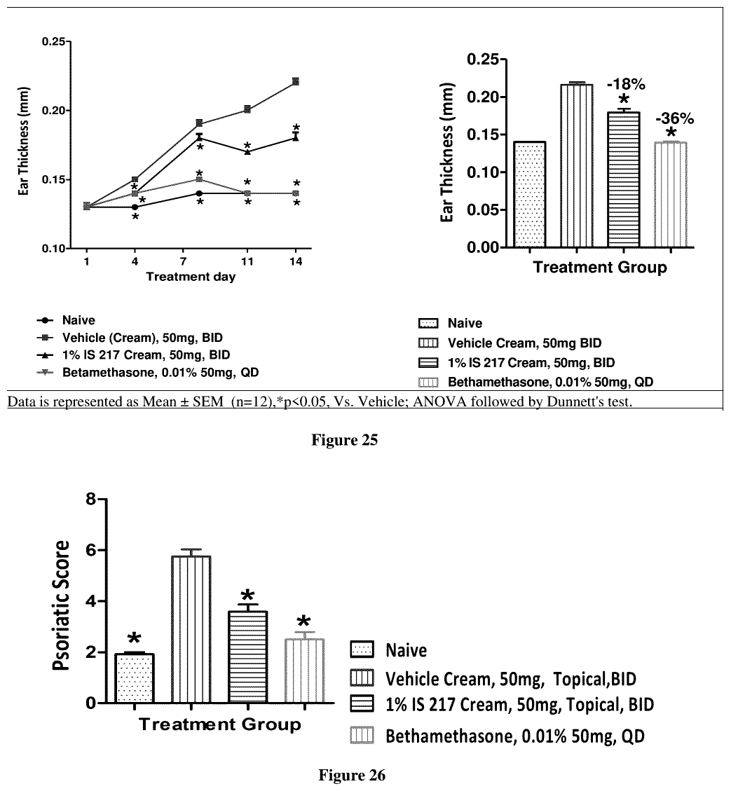

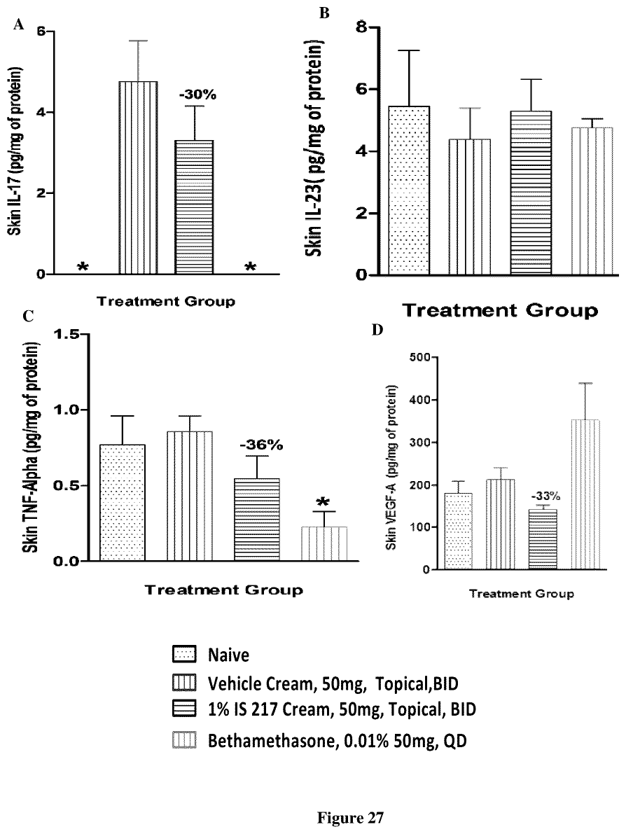

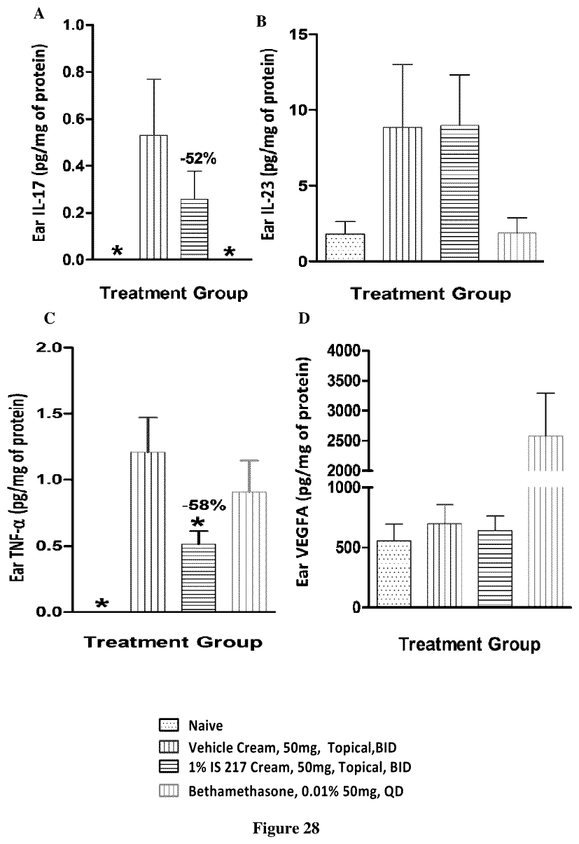

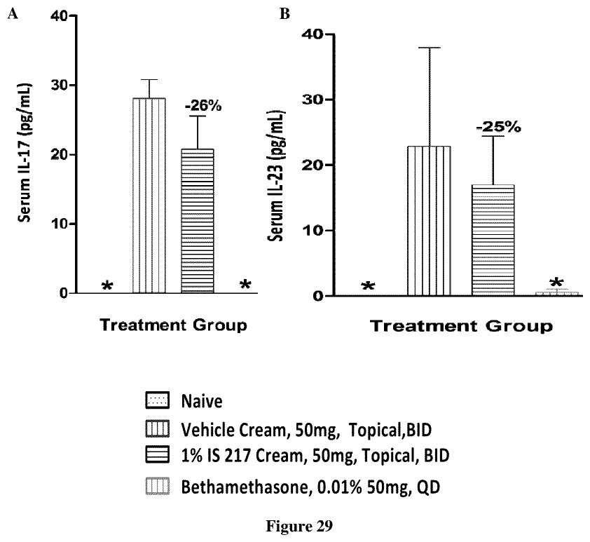

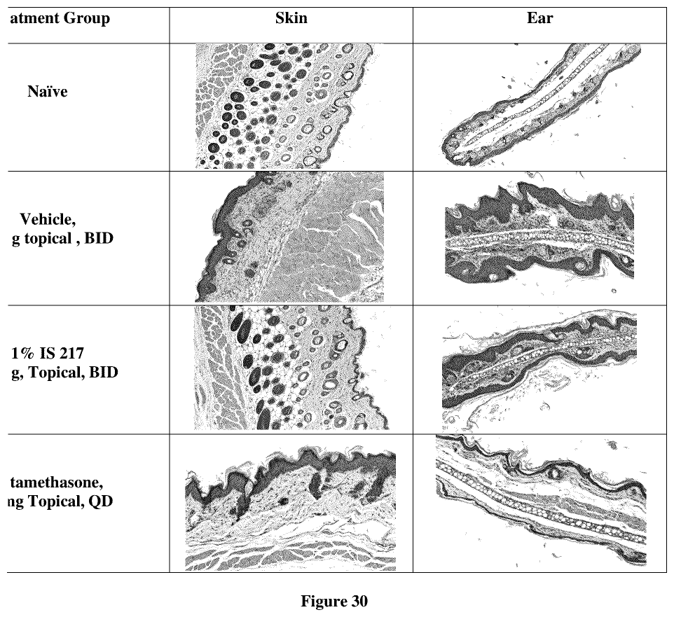

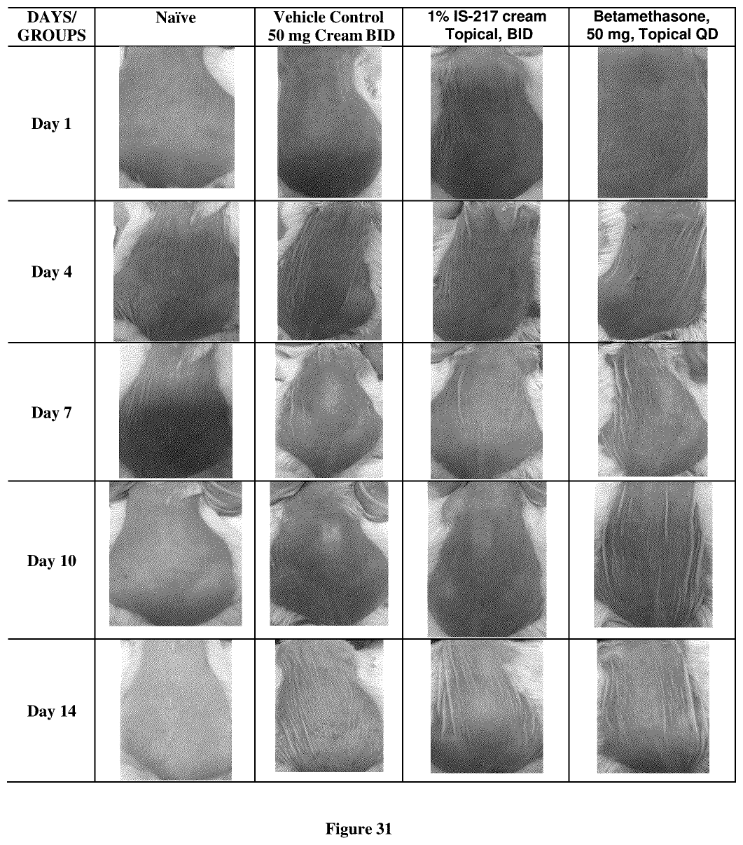

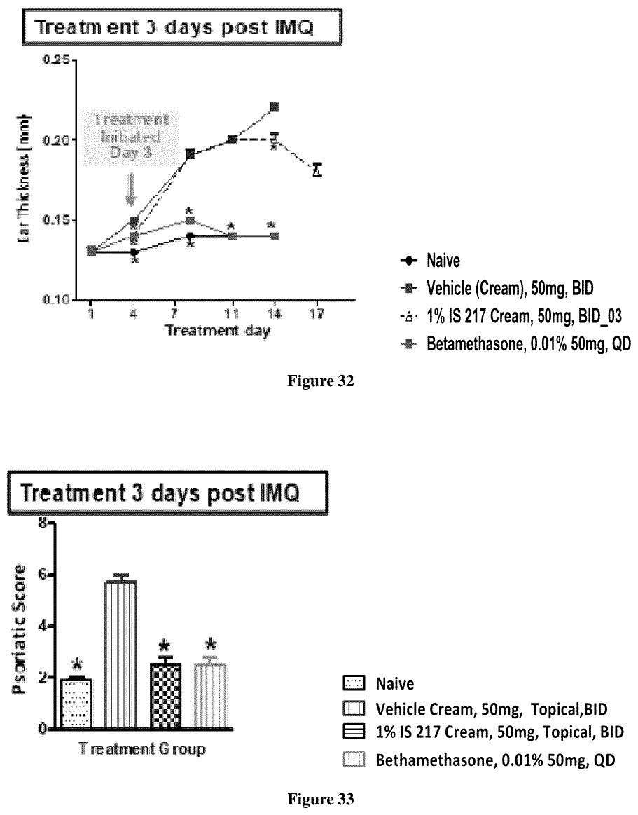

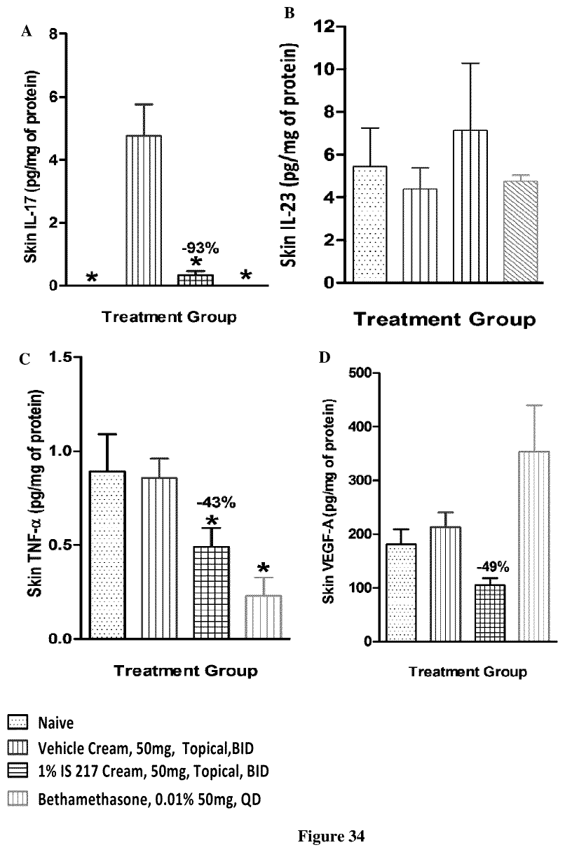

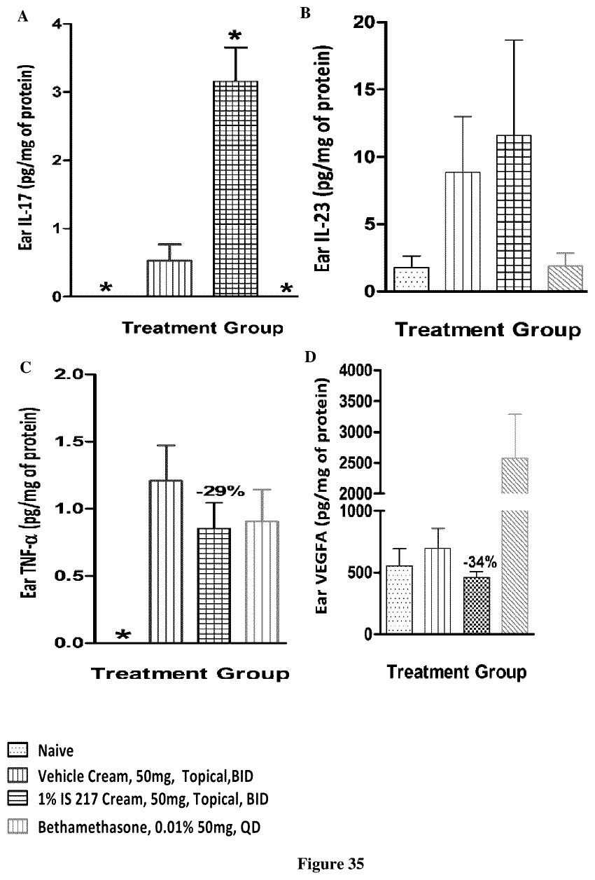

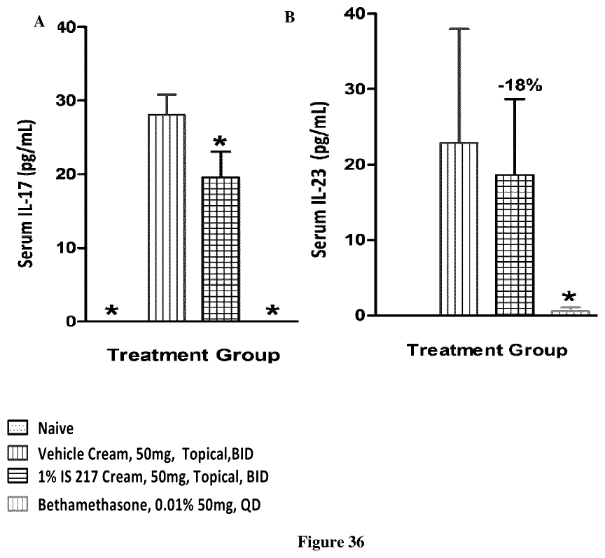

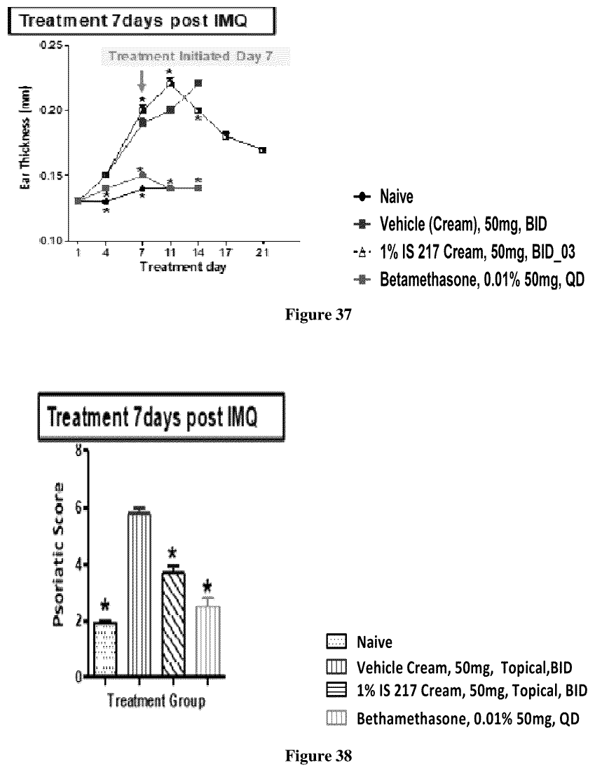

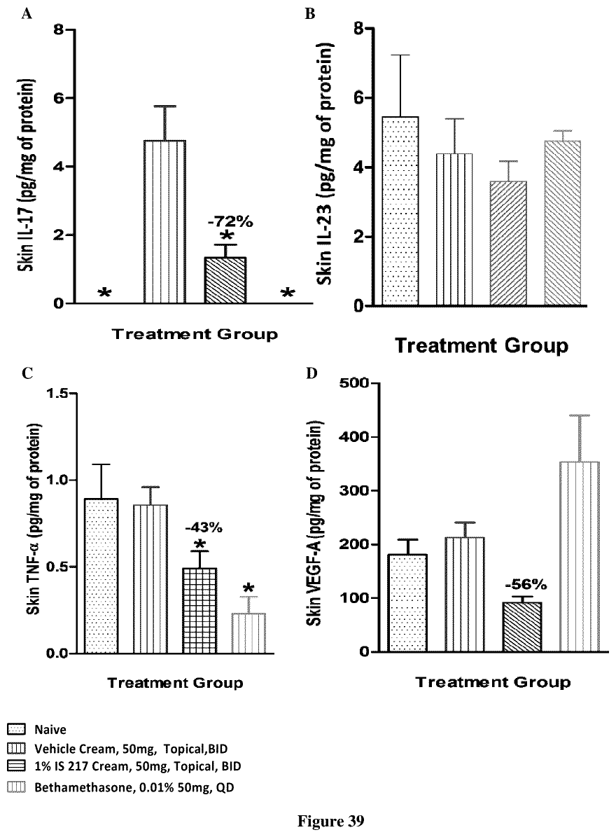

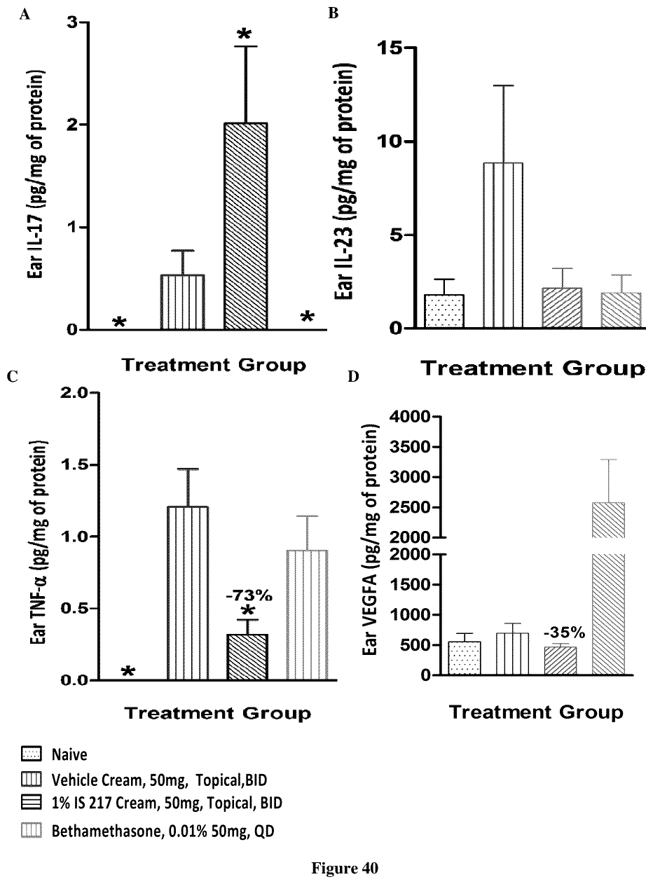

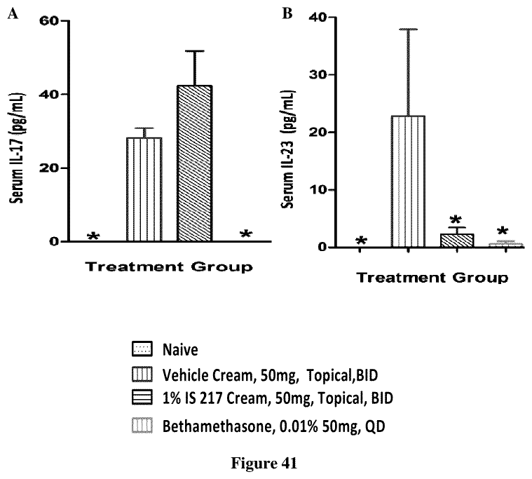

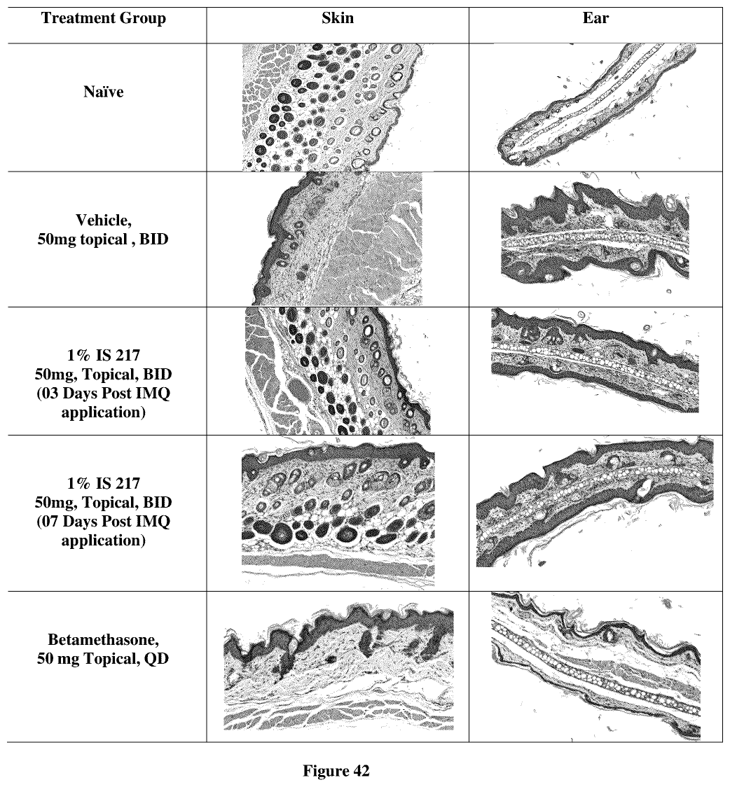

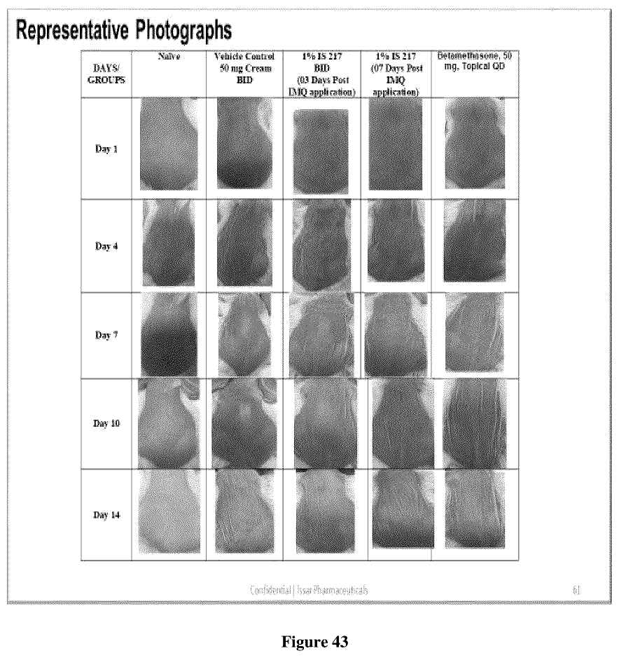

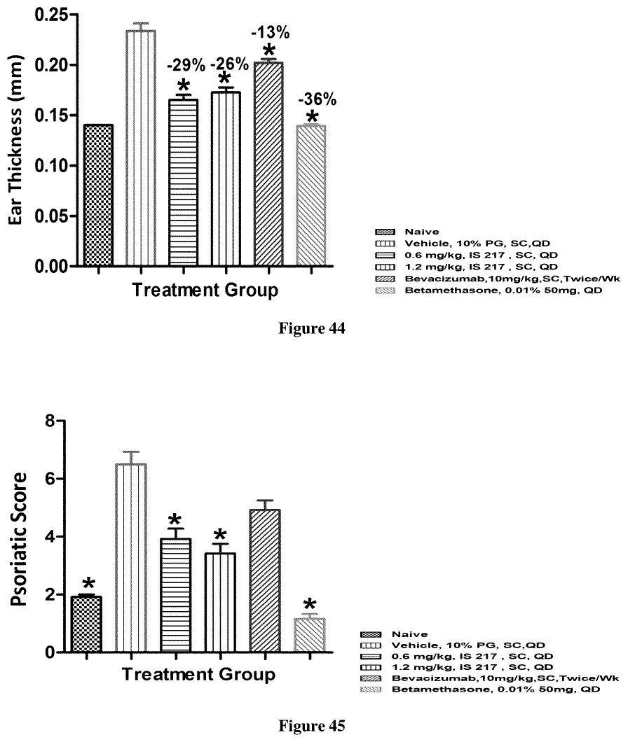

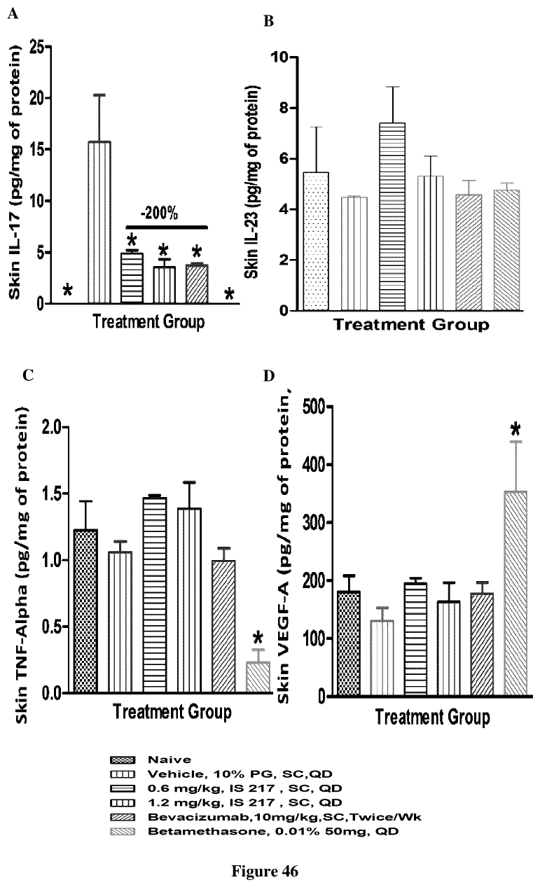

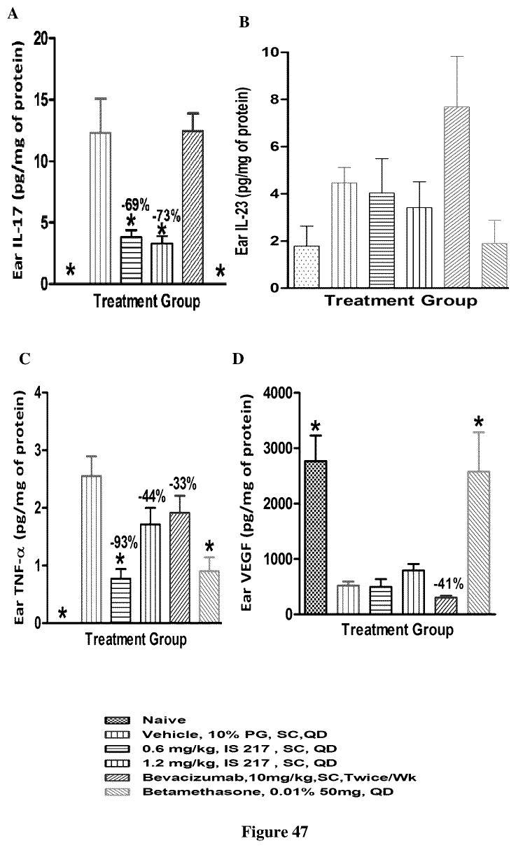

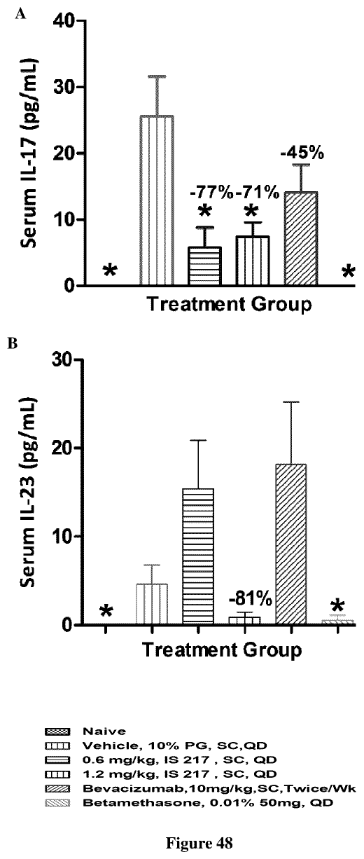

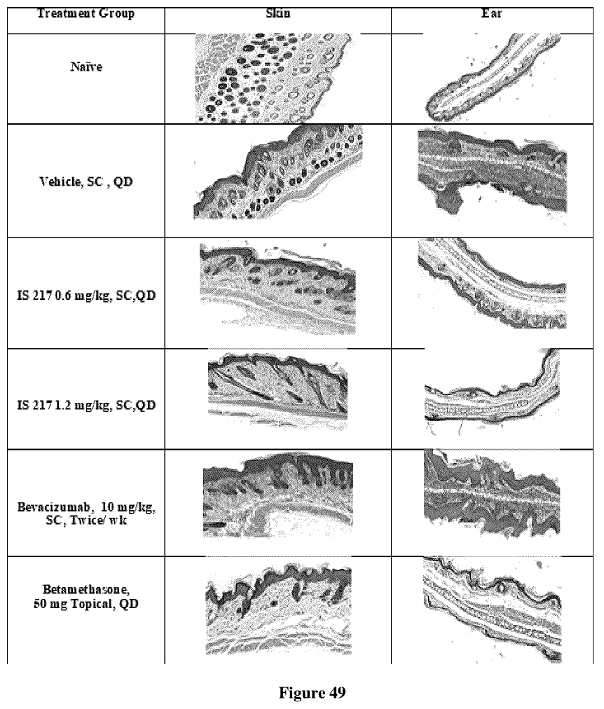

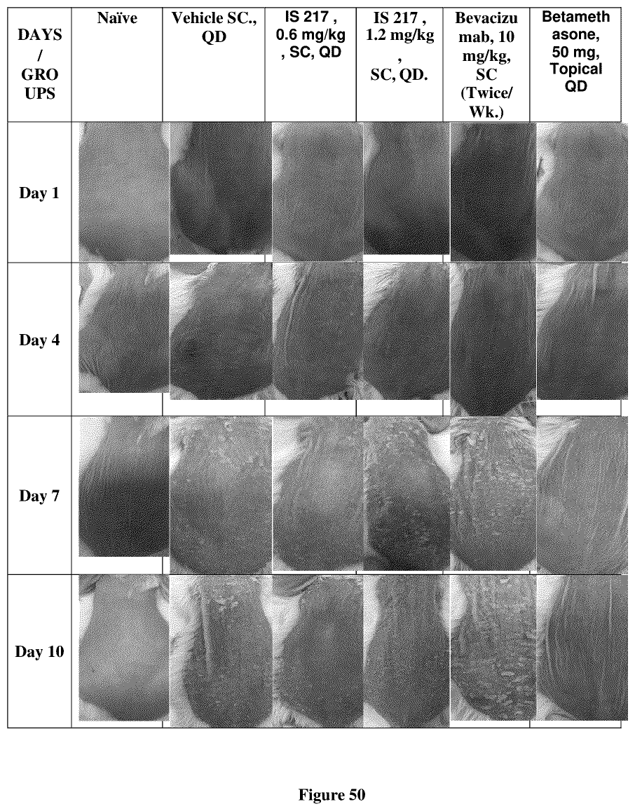

22.7: Prophylactic Treatment of Psoriasis with 1% IS217 Topical Application:
1% IS217 topical application of test compound significantly decreased ear thickness, which clearly indicates decrease in ear inflammation and edema. This was also supported by significant decrease in scaling, erythema and skin thickness (FIG. 25).
1% IS217 topical treatment group showed significant decrease in psoriatic score, ear weight, ear edema and significant decrease in spleen weight was observed that indicates decrease in systemic inflammation (FIG. 26).
1% IS217 topical treatment has inhibitory effect on Th17 pathway which is evident by decreased IL-17 and IL-23 level in ear, skin and serum matrix in 1% IS217 prophylactic treatment group compared to vehicle treatment. Elevated level of Th1 related cytokines such as TNF-α were decreased in ear and skin homogenates as well as in serum matrix. VEGFA level were markedly reduced in skin homogenate sample of 1% IS217 treatment group, however there was no change observed in ear homogenate samples compared with the vehicle treatment (FIGS. 27 to 29).
Characteristic psoriasis like symptoms such as epidermal hyperplasia, parakeratosis in skin and ear were significantly reduced in 1% IS217 topical group (FIG. 30), with representative photographs of all treatment groups (FIG. 31).
   22.8: Semi Therapeutic Treatment of Psoriasis with 1% IS217 Topical Application:
1% IS217 compound treatment was initiated 03 post-IMQ applications. Ear inflammation started developing but was significant compared to naïve group.
1% IS217 topical application of test compound decreased ear thickness, which clearly indicated decrease in ear inflammation and edema. This was also supported by significant decrease in scaling, erythema and skin thickness post topical application of 1% IS217 semi therapeutic mode (FIG. 32).
1% IS217 semi therapeutic mode of treatment showed significant decrease in psoriatic score and ear weight that indicates decrease in ear edema (FIG. 33).
1% IS217 topical treatment has inhibitory effect on Th17 pathway which is evident by decreased IL-17 and IL-23 level in skin and serum matrix. However, there is significant increase in IL-17 level was observed in ear homogenate samples compared to vehicle control. Elevated level of Th1 related cytokines such as TNF-α were decreased in skin and ear homogenates (FIGS. 34 to 36).
VEGFA level were markedly reduced in skin and ear homogenate 1% IS217 treatment group compared to vehicle treatment.
Characteristic psoriasis like symptoms such as epidermal hyperplasia, parakeratosis in skin were significantly reduced in 1% IS217 topical semi therapeutic treatment group (FIG. 42), with representative photographs of all treatment groups (FIG. 43).
   22.9: Therapeutic Treatment of Psoriasis with 1% IS217 Topical Application
1% IS217 compound treatment was initiated 07 post-IMQ application, ear thickness was significantly higher compared to naïve at the time of initiation of treatment.
1% IS217 topical application of test compound decreased ear thickness, which indicates decrease in ear inflammation and edema. This was also supported by significant decrease in scaling and erythema post topical application of 1% IS217 therapeutic mode (FIG. 37).
1% IS217 therapeutic mode of treatment showed significant decrease in psoriatic score and ear weight that indicates decrease in ear edema (FIG. 38).
1% IS217 topical therapeutic treatment decreased IL-17 level only in skin samples.
Serum and ear IL-23 level were reduced in 1% IS217 therapeutic treatment group compared to vehicle treatment, whereas no significant change was observed in skin in IL-23 level. Elevated level of Th1 related cytokines such as TNF-α were decreased in skin and ear homogenates (FIGS. 39 to 41).
VEGFA level were markedly reduced in skin and ear homogenate 1% IS217 therapeutic treatment group compared to vehicle treatment.
Characteristic psoriasis like symptoms such as epidermal hyperplasia, parakeratosis in skin were reduced in 1% IS217 topical therapeutic treatment group (FIG. 42), with representative photographs of all treatment groups (FIG. 43).
Overall, 1% IS217 topical application demonstrated efficacy in IMQ induced psoriasis model and are comparable to the standard drug (Betamethasone).
   22.10: Therapeutic Treatment of Psoriasis with IS217 at 0.6 mg/kg and 1.2 mg/kg on Subcutaneous Administration
IS217 at 0.6 mg/kg and 1.2 mg/kg SC treatment group significantly decreased ear thickness, which clearly indicated decrease in ear inflammation and edema. This was also supported by decrease in scaling, erythema and skin thickness (FIG. 44).
IS217 at 0.6 mg/kg and 1.2 mg/kg SC, treatment group showed significant decrease in psoriatic score, ear weight that indicates decrease in ear edema (FIG. 45).
Decrease in spleen weight was observed that indicates decrease in systemic inflammation.
IS217 at 0.6 mg/kg and 1.2 mg/kg SC treatment group has inhibitory effect on Th17 pathway which is evident by significant decrease in IL-17 level in ear, skin, and serum matrix.
Elevated level of Th1 related cytokine TNF-α was decreased in IS217 at 0.6 mg/kg and 1.2 mg/kg SC treatment group in ear homogenates samples (FIGS. 46 to 48).
Characteristic psoriasis like symptoms such as epidermal hyperplasia, parakeratosis in skin were reduced by using 1.2 mg/kg treatment groups, whereas only epidermal hyperplasia was reduced in 0.6 mg/kg treatment group.
Epidermal hyperplasia, parakeratosis, leukocyte infiltration was significantly decreased in IS217 at 0.6 mg/kg and 1.2 mg/kg treatment groups compared to vehicle (FIG. 49), with representative photographs of all treatment groups (FIG. 50).
Overall, IS217 at 0.6 mg/kg and 1.2 mg/kg SC demonstrated efficacy in IMQ-induced psoriasis model and are comparable to the standard drug (Bevacizumab).

Conclusions: These results demonstrate that IS217 is targeting VEGFR-2 by downregulating p38 kinases and pro-inflammatory IL-17 and IL-23 secretion and has inhibitory effect on Th17 pathway. Taken together; IS217 molecule is the possible drug molecule to treat inflammatory skin diseases such as psoriasis by subcutaneous and topical administration of an antiangiogenic compound.

### Example 23: Stability of IS217 API (Drug Substance) and IS217 1% Cream (Drug Product) Under Accelerated Conditions and Under Long-Term Storage Stability of Drug Substance

### Introduction

The purpose of stability testing is to provide evidence on how the quality of a drug substance or drug product varies with time under the influence of a variety of environmental factors such as temperature, humidity, etc. and to establish a re-test period for the drug substance or a shelf life for the drug product and recommended storage conditions. The study was conducted according to the WHO and Schedule-Y guidelines [62-63].

### Container Closure System

The drug substance is a lyophilized white fluffy powder, and it was filled at sterile condition in glass injection vial which has airlock-rubber capping. This container closure system is the same as or simulates the packaging proposed for storage and distribution.

### Specifications

The study specifications were designed and conducted according to WHO and schedule Y guidelines and the details of the same are described in below section.

### Test Attributes and Acceptance Criteria:

This study has included testing of those attributes of the drug substance that are susceptible to change during storage and are likely to influence quality, safety and/or efficacy. The testing was covered, as appropriate, the physical and chemical attributes.

In this stability testing of drug substance, the below attributes were investigated
which are potentially susceptible to change during storage
which are likely to influence quality, safety and/or efficacy.

The following test attributes have been selected according to above said guidelines.
Appearance; odour; colour; colour of solution (when the drug substance is soluble in solvent); clarity of solution; degradation and assessment of container closure system

TABLE 11
Test attributes of the drug substance
monitored and their acceptance criteria

| S. No. | Test attributes | Acceptance criteria |
|---|---|---|
| 1 | Appearance | White to off-white substance |
| 2 | Odour | Odour less |
| 3 | Colour | White |
| 4 | Colour of the solution | Clear solution |
| 5 | Clarity of the solution | Clear transparent |
| 6 | Degradation | Not more than 5% |
| 7 | Assessment of | Appearance unchanged |
| | container closure system | |

### Testing Frequency

Testing frequency for long-term studies have been designed as per schedule Y. The long-term stability studies have been conducted on 1, 3, 6, 9, 12, 18, and 24 months.

### Storage Conditions

The studies were conducted under appropriate storage conditions (with appropriate tolerances) that test drug substance thermal stability and its sensitivity to moisture. The drug substance is intended to store for 24 months. Accordingly, long term stability studies were done for 24 months. The details of the storage conditions have been furnished hereunder.

TABLE 12
Sample storage conditions and frequency
of testing as per schedule Y requirement

| | | Storage conditions | |
|---|---|---|---|
| | | Condition1 | Condition2 |
| | | (accelerated | (long-term |
| Sr. No. | Parameter | studies 1) | studies) |
| 1. | Temperature | 5 ± 3° C. | −20 ± 5° C. |
| 2. | Relative | 60 ± 5% | 60 ± 5% |
| 3. | Humidity | 1, 2, 3, 4, 5 and 6 | 1, 3, 6, 9, 12, 18 and 24 |
| | Frequency of | | |
| | testing (months) | | |

### Stability Commitment

The stability data was generated on pilot scale batches. It has been proposed that the drug substance re-test period for 24 months.

### Evaluation of Results

### 23.1: Acceleratory Stability Evaluation (Condition 1)

The attributes were not changed during the acceleratory stability testing period. The details of the same are furnished below.
Appearance, color, odor, color of solution, clarity of solution and assessment of container closure system

Drug substance was observed in its natural color and no change was observed in all other attributes during the study period. All other attributes were noted within acceptance range during the study period.

TABLE 13
Evaluation of internal change in attributes during
the acceleratory (Condition 1) study period

| | 1^{st} | 2^{nd} | 3^{rd} | 6^{th} |
|---|---|---|---|---|
| Attribute | month | month | month | month |
| Appearance | No change | No change | No change | No change |
| Color | No change | No change | No change | No change |
| Odor | No change | No change | No change | No change |
| Color of | No change | No change | No change | No change |
| Solution | | | | |
| Clarity of | No change | No change | No change | No change |
| solution | | | | |
| Assessment of | No change | No change | No change | No change |
| container closure | | | | |
| system | | | | |

### 23.2: Long Term Stability Evaluation (Condition 2)

The attributes described in above section were not changed during the long-term stability study period. The details of the same have been furnished below table.
Appearance, color, odor, color of solution, clarity of solution and assessment of container closure system

All mentioned attributes in below table were observed within the acceptance range.

TABLE 14
Evaluation of internal change in attributes
during the long-term study period

| | 1^{st} | 2^{nd} | 3^{rd} | 6^{th} |
|---|---|---|---|---|
| Attribute | month | month | month | month |
| Appearance | No change | No change | No change | No change |
| Color | No change | No change | No change | No change |
| Odor | No change | No change | No change | No change |
| Color of | No change | No change | No change | No change |
| Solution | | | | |
| Clarity of | No change | No change | No change | No change |
| solution | | | | |
| Assessment of | No change | No change | No change | No change |
| container closure | | | | |
| system | | | | |

### 23.3: Accelerated Stability Study Results

Name of the product: IS217; Batch size: 200 m·mol; Storage conditions: 5±3° C.

TABLE 15
Experimental results of accelerated stability study results at storage conditions of 5 ± 3° C.

| | | | Identification | | | | | | |
|---|---|---|---|---|---|---|---|---|---|
| | | | (By HPLC) | | | | | | |
| | | | Retention | | | | | | |
| | | | time of main | | | | | | |
| | | | peak in the | | | | | | |
| | | | chromatogram | | Water | API | | | |
| | | | of the sample | | Content | calculated | Related | | |
| | | | preparation | Identification | (By Karl | on the | substances | | |
| | | | should correspond | Mass | Fischer- | anhydrous basis | (By HPLC) | | |
| | Appearance | | to that of | Spectrum | Titrator) | Not less than | Any | | |
| | white to | | standard | (By LC/MS) | Not be | 97.0% and not | Max. | Total | |
| Interval | off white | Solubility | preparation | Should be | more than | more than | Impurity | Impurity | |
| In | amorphous | Soluble | as obtained | 1242.6 ± 2 | 5.0% | 102.0% | NMT | NMT | |
| Months | powder | in water. | in the assay. | M.W. | w/w | (OAB) | 1.5% | 3.0% | Remark |
| Initial | White | Complies | Complies | 1242.7 M.W. | 4.66% w/w | 99.28% | 0.60% | 0.72% | Complies |
| | amorphous | | | | | | | | |
| | powder. | | | | | | | | |
| 1 M | White | Complies | Complies | 1242.7 M.W. | 4.61% w/w | 99.36% | 0.52% | 0.63% | No |
| | amorphous | | | | | | | | change |
| | powder. | | | | | | | | |
| 2 M | White | Complies | Complies | 1242.7 M.W. | 4.55% w/w | 99.25% | 0.53% | 0.76% | No |
| | amorphous | | | | | | | | change |
| | powder. | | | | | | | | |
| 3 M | White | Complies | Complies | 1242.7 M.W. | 4.60% w/w | 99.06% | 0.59% | 0.94% | No |
| | amorphous | | | | | | | | change |
| | powder. | | | | | | | | |
| 6 M | White | Complies | Complies | 1242.7 M.W. | 4.62% w/w | 99.22% | 0.43% | 0.78% | No |
| | amorphous | | | | | | | | change |
| | powder. | | | | | | | | |

Conclusion: IS217 API is found to be stable for 6 months at accelerated condition as no significant change from initial analysis has been observed. 23.4: Long term stability study results Name of the Product: IS217; Batch size: 200 m·mol; Storage Conditions: −20±5° C.

TABLE 16
Experimental results of long-term stability study results at storage conditions of −20 ± 5° C.

| | | | Identification | | | | | | |
|---|---|---|---|---|---|---|---|---|---|
| | | | (By HPLC) | | | | | | |
| | | | Retention | | | | | | |
| | | | time of main | | | | | | |
| | | | peak in the | | | | | | |
| | | | chromatogram | | | API | | | |
| | | | of the sample | | Water | calculated | Related | | |
| | | | preparation | Identification | Content | on the | substances | | |
| | | | should correspond | Mass | (By Karl | anhydrous basis | (By HPLC) | | |
| | Appearance | | to that of | Spectrum | Fischer- | Not less than | Any | | |
| | white to | | standard | (By LC/MS) | Titrator) | 97.0% and not | Max. | Total | |
| Interval | off white | Solubility | preparation | Should be | Not be | more than | Impurity | Impurity | |
| in | amorphous | Soluble | as obtained | 1242.6 ± 2 | more than | 102.0% | NMT | NMT | |
| months | powder | in water. | in the assay. | M.W. | 5.0% w/w | (OAB) | 1.5% | 3.0% | Remark |
| Initial | White | Complies | Complies | 1242.7 M.W. | 4.66% w/w | 99.28% | 0.60% | 0.72% | Complies |
| | amorphous | | | | | | | | |
| | powder. | | | | | | | | |
| 1 M | White | Complies | Complies | 1242.7 M.W. | 4.89% w/w | 98.98% | 0.64% | 0.85% | No |
| | amorphous | | | | | | | | change |
| | powder. | | | | | | | | |
| 2 M | White | Complies | Complies | 1242.7 M.W. | 4.83% w/w | 99.14% | 0.64% | 0.85% | No |
| | amorphous | | | | | | | | change |
| | powder. | | | | | | | | |
| 3 M | White | Complies | Complies | 1242.7 M.W. | 4.52% w/w | 99.15% | 0.72% | 0.85% | No |
| | amorphous | | | | | | | | change |
| | powder. | | | | | | | | |
| 6 M | White | Complies | Complies | 1242.7 M.W. | 4.99% w/w | 99.40% | 0.35% | 0.61% | No |
| | amorphous | | | | | | | | change |
| | powder. | | | | | | | | |

Conclusion: IS217 API is found to be stable for 6 months at long term stability condition (of −20±5° C.) as no significant change from initial analysis has been observed.

Summary and Conclusion: For drug substances or products intended for storage in a freezer, the retest period or shelf life should be based on long-term data [65].

The ICH guidelines and schedule Y provides that for a drug substances intended for storage in a refrigerator or freezer, the drug should pass long term stability storage conditions and accelerated storage study conditions for minimum time period [65]. Based on all the above data illustrated in Tables 13 to 16 it can be concluded that the drug substance is stable at refrigerator (5±3° C.) conditions for 24 months.

### 23.5: Stability of Drug Product

The study was done in-house and completed report with signatures and HPLC chromatograms is kept in Guard file as confidential.

### Introduction

The purpose of stability testing is to provide evidence on how the quality of a drug product varies with time under the influence of a variety of environmental factors such as temperature, humidity, etc. and to establish a re-test period for the drug substance or a shelf life for the drug product and recommended storage conditions. The study was conducted according to the WHO and schedule-Y guidelines [62-63].

### General Properties

IS217 cream (1%) is white oil in water type emulsion containing IS217, active pharmaceutical ingredient (API). The drug substance, IS217, is a white amorphous lyophilized powder and is the Applicant's designation for a 11 amino acid peptide under development for the treatment of psoriasis, antimicrobial agent etc.

### Composition of IS217 1% Cream:

The list of components, with its quantities, usages in process and their quality standers were furnished as given in Table 1.

### Selection of Batches

The drug product was manufactured by Issar Pharma. Three batches were manufactured at pilot scale level by the same synthetic route as, and using a method of manufacture and procedure that simulates the final process to be used for, production batches. The overall quality of the batches of drug product placed on these stability studies will be representative of the quality of the material to be made on a production scale.

### Container Closure System

A soft squeezable plastic tube will be used as primary packing material. The tube is a cylindrical, hollow piece with a rounded shape design. The tube should be fitted with a screw cap closure that minimizes microbial contamination and that reveals whether the container has ever been opened.

### Specifications

The study specifications were designed and conducted according to WHO and schedule Y guidelines and the details of the same were described in below section.

### Test Attributes and Acceptance Criteria:

This study has included testing of those attributes of the drug product that are susceptible to change during storage and are likely to influence quality, safety and/or efficacy. The testing was covered, as appropriate, the physical and chemical attributes. In this stability testing of drug product the below attributes were investigated
which are potentially susceptible to change during the course of storage
which are likely to influence quality, safety and/or efficacy.

The following test attributes have been selected according to above said guidelines.
appearance; odour; colour; degradation; assessment of container closure system.

TABLE 17
Test attributes studied and their acceptance criteria

| Sr. No | Test attributes | Acceptance criteria |
|---|---|---|
| 1 | Appearance | White Colour Cream |
| 2 | Odour | Odour less |
| 3 | Colour | White |
| 4 | Degradation | Not more than 5% |
| 5 | Assessment of container | Appearance unchanged |
| | closure system | |

### Testing Frequency

Testing frequency for long term studies have been designed as per schedule Y. The long term stability studies have been conducted on 1, 3, 6, 9, 12, 18, and 24 month.

### Storage Conditions

The studies were conducted under appropriate storage conditions (with appropriate tolerances) that test drug product thermal stability and its sensitivity to moisture. The drug product is intended to store for 24 months. Accordingly, long term stability studies were done for 24 months. The details of the storage conditions have been furnished hereunder.

TABLE 18
Sample storage conditions and frequency of testing as per schedule Y requirement

| | | Storage conditions | | |
|---|---|---|---|---|
| | | Condition1 | Condition2 | Condition3 |
| | | (accelerated | (long term | (long term |
| Sr. No. | Parameter | studies 1) | Studies) | studies) |
| 1. | Temperature | 40° C. ± 2° C. | 30° C. ± 2° C. | 25° C. ± 2° C. |
| 2. | Relative | 75% RH ± 5% | 65% RH ± 5% | 65% RH ± 5% |
| 3. | Humidity | 1, 2, 3, 4, 5and 6 | 1, 3, 6, 9, 12, 18 | 1, 3, 6, 9, 12, 18 and |
| | Frequency of | | and 24 | 24 |
| | testing (months) | | | |

### Stability Commitment

The stability data was generated on pilot scale batches. It has been proposed that the drug substance re-test period for 24 months. Thus, the long term stability data of three batches was covered the proposed period.

### Evaluation of Results

### 23.5.1: Acceleratory Stability Evaluation (Condition 1)

The attributes were not changed during the acceleratory stability testing period. The details of the same were furnished below.
appearance, color, odor, color of solution, clarity of solution and assessment of container closure system

Drug substance was observed in its natural color and No change was observed in all other attributes during the study period. All other attributes were noted within acceptance range during the study period.

TABLE 19
Evaluation of internal change in attributes during
the acceleratory (condition 1) study period.

| | 1^{st} | 2^{nd} | 3^{rd} | 6^{th} |
|---|---|---|---|---|
| Attribute | month | month | month | month |
| Appearance | No change | No change | No change | No change |
| Color | No change | No change | No change | No change |
| Odor | No change | No change | No change | No change |
| Assessment of | No change | No change | No change | No change |
| container closure | | | | |
| system | | | | |

### 23.5.2: Long Term Stability Evaluation (Condition 2)

The attributes described in above section were not changed during the long-term stability study period. The details of the same have been furnished below table.
Appearance, color, odor, color of solution, clarity of solution and assessment of container closure system

All mentioned attributes in below table were observed within the acceptance range.

TABLE 20
Evaluation of internal change in attributes during the long term study period
at 30° C. ± 2° C. and 65% RH ± 5%

| | 1^{st} | 2^{nd} | 3^{rd} | 6^{th} | 9^{th} | 12^{th} | 18^{th} | 24^{th} |
|---|---|---|---|---|---|---|---|---|
| Attribute | month | month | month | month | month | month | month | month |
| Appearance | No | No | No | No | No | No | No | No |
| | change | change | change | change | change | change | change | change |
| Color | No | No | No | No | No | No | No | No |
| | change | change | change | change | Change | Change | Change | change |
| Odor | No | No | No | No | No | No | No | No |
| | change | change | change | change | change | change | change | change |
| Assessment | No | No | No | No | No | No | No | No |
| of container | change | change | change | change | Change | Change | Change | Change |
| closure | | | | | | | | |

### 23.5.3: Long Term Stability Evaluation (Condition 3)

The attributes described in above section were not changed during the long term stability study period. The details of the same have been furnished below table.
appearance, color, odor, color of solution, clarity of solution and assessment of container closure system

All mentioned attributes in below table were observed within the acceptance range.

TABLE 21
Evaluation of internal change in attributes during the long term study period
at 25° C. ± 2° C. and 65% RH ± 5%

| | 1^{st} | 2^{nd} | 3^{rd} | 6^{th} | 9^{th} | 12^{th} | 18^{th} | 24^{th} |
|---|---|---|---|---|---|---|---|---|
| Attribute | month | month | month | month | month | month | month | month |
| Appearance | No | No | No | No | No | No | No | No |
| | change | change | change | change | change | change | change | change |
| Color | No | No | No | No | No | No | No | No |
| | change | change | change | change | Change | Change | Change | change |
| Odor | No | No | No | No | No | No | No | No |
| | change | change | change | change | change | change | change | change |
| Assessment | No | No | No | No | No | No | No | No |
| of container | change | change | change | change | Change | Change | Change | Change |
| closure | | | | | | | | |
| system | | | | | | | | |

### 23.6: Accelerated Stability Study Results

Name of the product: S217 1% Cream; Batch size: 2.0 kg; Pack size: 50 gi; storage conditions: 40±2° C.

TABLE 22
Results of accelerated stability study at storage conditions of 40 ± 2° C.

| | | Stability tests and limits | | | | | |
|---|---|---|---|---|---|---|---|
| | | | | | | | Microbiological |
| | | | | | | | Limits |
| | | | | | | | Total aerobic |
| | | | | | | | Count: NMT 100 |
| | | | | | | | cfu/ml Yeast and |
| | | | | | | | Molds: 10 cfu/ml |
| | | | | | | | Absence of |
| | | | | | | | E. coli, S. aureus, |
| | | | | | | Assay (By | P. aeruginosa, |
| | | | | | | HPLC) | Salmonella, Bile- |
| | | | | pH | Viscosity | NLT 90.0% | tolerant gram |
| | | Appearance | Identification | (at 25° C.) | Not less | and NMT | negative bacteria, |
| Sr. | Stability | White color | (By HPLC) | Between | than 80.0% | 110.0% of | clostridia and |
| No | station | cream | Complies | 4.0 to 6.0 | Torque | label claim | candidia albicans |
| 1 | Initial | White color | Complies | 5.90 | 89% | 100.86% | Complies |
| | | cream | | | | | |
| 2 | 1 M | White color | Complies | 5.94 | 87% | 101.20% | Complies |
| | | cream | | | | | |
| 3 | 2 M | White color | Complies | 5.93 | 94% | 99.39% | Complies |
| | | cream | | | | | |
| 4 | 3 M | White color | Complies | 5.90 | 92% | 99.85% | Complies |
| | | cream | | | | | |
| 5 | 6 M | White color | Complies | 6.0 | 88% | 101.15% | Complies |
| | | cream | | | | | |

Conclusion: IS217 1% Cream is found to be stable for 6 months at accelerated condition as no significant change from initial analysis has been observed.

### 23.7: Long Term Stability Study Results Condition-02

Name of the product: e217 1% Cream; Batch size: 2.0 kg; Pack size: 50 gm; Storage conditions: 30±2° C.

TABLE 23
Results of long term stability studies (condition 2) at storage conditions of 30 ± 2° C.

| | | Stability tests and limits | | | | | |
|---|---|---|---|---|---|---|---|
| | | | | | | | Microbiological |
| | | | | | | | limits |
| | | | | | | | Total aerobic |
| | | | | | | | Count: NMT |
| | | | | | | | 100 cfu/ml |
| | | | | | | | Yeast and |
| | | | | | | | Molds: 10 cfu/ml |
| | | | | | | | Absence of |
| | | | | | | | E. coli, S. aureus, |
| | | | | | | | P. aeruginosa, |
| | | | | | | Assay (By | Salmonella, |
| | | | | | | HPLC) | Bile-tolerant |
| | | | | pH | Viscosity | NLT 90.0% | gram negative |
| | | Appearance | Identification | (at 25° C.) | Not less | and NMT | bacteria, |
| Sr. | Stability | White color | (By HPLC) | Between | than 80.0% | 110.0% of | clostridia and |
| No | station | cream | Complies | 4.0 to 6.0 | Torque | label claim | candidia albicans |
| 1 | Initial | White color | Complies | 5.90 | 89% | 100.86% | Complies |
| | | cream | | | | | |
| 2 | 1 M | White color | Complies | 6.11 | 87% | 99.31% | Complies |
| | | cream | | | | | |
| 3 | 2 M | White color | Complies | 6.32 | 94% | 95.45% | Complies |
| | | cream | | | | | |
| 4 | 3 M | White color | Complies | 6.12 | 92% | 95.95% | Complies |
| | | cream | | | | | |
| 5 | 6 M | White color | Complies | 6.23 | 88% | 99.20% | Complies |
| | | cream | | | | | |
| 6 | 9 M | White color | Complies | 5.94 | 89% | 100.44% | Complies |
| | | cream | | | | | |
| 7 | 12 M | White color | Complies | 5.93 | 87% | 100.69% | Complies |
| | | cream | | | | | |
| 8 | 18 M | White color | Complies | 5.98 | 94% | 96.89% | Complies |
| | | cream | | | | | |
| 9 | 24 M | White color | Complies | 6.0 | 92% | 99.61% | Complies |
| | | cream | | | | | |

Conclusion: IS217 1% Cream is found to be stable for 24 months at long term stability conditions as no significant change from initial analysis has been observed.

### 23.8: Long Term Stability Study Results Condition-023

Name of the product: IS217 1% Cream; Batch size: 2.0 kg; Pack size: 50 gm; Storage conditions: 25±2° C.

TABLE 24
Results of long term stability study (condition 023) at storage conditions of 25 ± 2° C.

| | | Stability tests and limits | | | | | |
|---|---|---|---|---|---|---|---|
| | | | | | | | Microbiological |
| | | | | | | | Limits |
| | | | | | | | Total aerobic |
| | | | | | | | Count: NMT |
| | | | | | | | 100 cfu/ml |
| | | | | | | | Yeast and |
| | | | | | | | Molds: 10 cfu/ml |
| | | | | | | | Absence of |
| | | | | | | | E. coli, S. aureus, |
| | | | | | | | P. aeruginosa, |
| | | | | | | Assay (By | Salmonella, |
| | | | | | | HPLC) | Bile-tolerant |
| | | | | pH | Viscosity | NLT 90.0% | gram negative |
| | | Appearance | Identification | (at 25° C.) | Not less | and NMT | bacteria, |
| Sr. | Stability | White color | (By HPLC) | Between | than 80.0% | 110.0% of | clostridia and |
| No | station | cream | Complies | 4.0 to 6.0 | Torque | label claim | candidia albicans |
| 1 | Initial | White color | Complies | 5.90 | 89% | 100.86% | Complies |
| | | cream | | | | | |
| 2 | 1 M | White color | Complies | 5.94 | 87% | 100.44% | Complies |
| | | cream | | | | | |
| 3 | 2 M | White color | Complies | 5.93 | 94% | 100.69% | Complies |
| | | cream | | | | | |
| 4 | 3 M | White color | Complies | 5.98 | 92% | 96.89% | Complies |
| | | cream | | | | | |
| 5 | 6 M | White color | Complies | 6.0 | 88% | 99.61% | Complies |
| | | cream | | | | | |
| 6 | 9 M | White color | Complies | 6.0 | 89% | 96.5% | Complies |
| | | cream | | | | | |
| 7 | 12 M | White color | Complies | 5.93 | 87% | 97.53% | Complies |
| | | cream | | | | | |
| 8 | 18 M | White color | Complies | 5.98 | 94% | 98.82% | Complies |
| | | cream | | | | | |
| 9 | 24 M | White color | Complies | 6.20 | 92% | 99.97% | Complies |
| | | cream | | | | | |

Conclusion: IS 217 1% Cream is found to be stable for 24 months at long term stability conditions as no significant change from initial analysis has been observed.

### Statements/Labelling

The drug substance is stable for 24 months at room temperature

The drug product, IS217 1% cream, is a synthetic peptide-based formulation and it is available in white cream form. It was intended that the re-test period of the drug product is 24 months at room temperature conditions. Hence long-term studies were carried out for 24 months at 30° C.±2° C./65% RH±5% RH. The batch was used for stability studies which are manufactured at pilot scale level by the same synthetic route as and using a method of manufacture and procedure that simulates the final process to be used for, production batches.

Attributes like appearance, color, odor and assessment of container closure system were not changed under long term storage condition for 24 months period. However, a slight degradation is observed during 24 months under long term storage condition, but this change was not crossed significant change limit. Rate of degradation and statistical regression line were observed within the acceptance criteria during the long term study period. It indicates that the drug product is stable at room temperature conditions for long duration.

Based on all the above it can be concluded that the drug substance is stable at room temperature condition for 24 months.

### Example 24: Establishing Mechanism of Action of IS217 in Inhibition of VEGF Interlinking with IL-17 and IL-23

Psoriasis is a chronic inflammatory skin disorder which is associated with increased cutaneous vascular endothelial growth factor (VEGF) expression and plays a vital role in the pathophysiology of psoriasis as VEGF influences vascular permeability and mediates pro-inflammatory activity by inducing vascular leakage. There are many references of both animal and human studies saying in non-involved, non-lesional skin significant over-expression of several VEGF isoforms was observed in psoriasis patients as compared with healthy skin of volunteers and most recent studies highlight the role of Th17 cells in psoriasis pathogenesis there is also evidence that in the course of the disease disturbed angiogenesis and skin inflammation are closely linked.

Several studies demonstrated that VEGF played an important role in psoriasis pathogenesis by linking angiogenesis and inflammation. However, the exact mechanisms of VEGF induced inflammation in psoriasis are unknown.

To support this here is Reference-1 of “VEGF Induces IL-23 Expression in Keratinocytes through p38 Signaling” Canavese M, Peric M, Dombrowski Y, Koglin S, Ruzicka T, et al. (2011). J Clin Exp Dermatol Res., states that in lesional psoriatic skin, VEGF and the pro-inflammatory cytokine IL-23 are both strongly expressed by epidermal keratinocytes. VEGF over-expression in cultured keratinocytes resulted in increased IL-23 and IL-6 mRNA transcript abundance and protein expression. At the same time VEGF over-expression strongly increased phosphorylation of p38 MAPK, CREB and HSp27 with conclusion that VEGF up-regulates pro-inflammatory IL-23 and IL-6 secretion through p38 MAPK in epidermal keratinocytes in psoriasis. Targeting VEGF and/or p38 MAPK could lead to novel anti-inflammatory treatments for this chronic skin disease.

Reference-2“Interleukin (IL)-22, IL-17, IL-23, IL-8, vascular endothelial growth factor and tumour necrosis factor-a levels in patients with psoriasis before, during and after psoralen-ultraviolet A and narrowband ultraviolet B therapy” S. Coimbra, H. Oliveira et al. (2010), British Association of Dermatologists 163, pp 1282-1290, states that cross-sectional studies of psoriatic patients shown that different cytokines and growth factors are enhanced and the study aimed to understand the role/relation of interleukin (IL)-22, IL-17, IL-23, IL-8, vascular endothelial growth factor (VEGF) and tumour necrosis factor (TNF)-α in psoriasis vulgaris, addressing their levels and changes before, during and after psoralen-ultraviolet A (PUVA) and narrowband ultraviolet B (NB-UVB) treatment and concluded that the levels of IL-22, IL-17, IL-23, IL-8, TNF-α and VEGF are reduced.

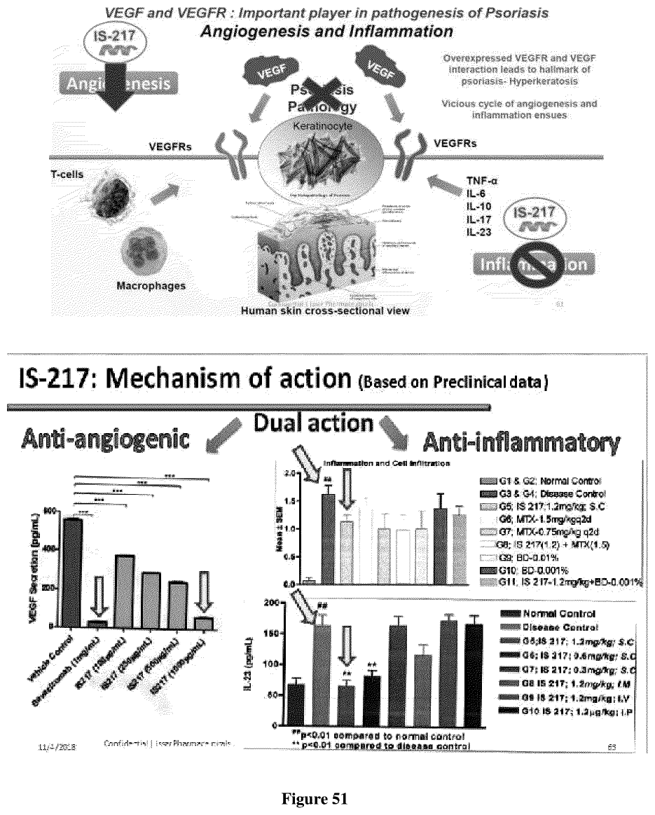

Conclusions: By the above observations and considering the references cited above it can be concluded that IS217 is targeting VEGF and/or p38 by downregulating pro-inflammatory IL-23 and IL-17 secretion (FIG. 51).

Details of source of materials employed for purpose of present invention are tabulated as below.

TABLE 25
Details of source of cell lines and animal
models employed in the present invention:

| Cell lines/mice models | Source | Country |
|---|---|---|
| HaCaT (Human Keratinocytes) | NCCS, Pune | India |
| THP-1 (Human Monocytic cell line) | NCCS, Pune | India |
| Human Endothelial Cells (Ea.hy.926) | ATCC, USA | USA |
| HUVECs (Human Umbilical Vein | ATCC, USA | USA |
| Endothelial Cells) | ATCC-pcs-100-013 | |
| | (HSN Code: 3001) | |
| Balb/c mice | National Institute of Nutrition, | India |
| | Hyderabad | |
| C57BL/6 mice | Liveon Biolabs Pvt Ltd | India |
| Balb/c mice | Vivo Bio Tech Limited, | India |
| | Hyderabad | |
| Balb/c mice | National Institute of Nutrition, | India |
| | Hyderabad | |

TABLE 26
Details of source of kits, chemicals and drugs employed in the present invention

| Chemical/ Drug | Manufactured By | Batch No | Country |
|---|---|---|---|
| Methotrexate | Clearsynth | AI281K14 | USA |
| 0.01% Batamethasone | GlaxoSmith Kline | NB125 | India |
| cream (Betnovate ®) | Pharma Ltd. | | |
| 100 mg/4 ml | Avastin ® Hoffmann- | B7233B19 | USA |
| Bevacuzimab(Avastin ®) | La Roche Ltd. | | |
| Annexin-V reagent | EMD Millipore | 4500-0450 | USA |
| (Guava Technologies) | EMD Millipore | — | USA |
| Cell Cycle Kit (Guava | Thermo | 23227 | USA |
| Technologies) | | | |
| BCA Protein Estimation | Biovision | K106 | USA |
| Kit | | | |
| Caspase-3 colorimetric | R&D systems | DVE00 | USA |
| assay kit | | | |
| Human VEGF ELISA kit | Millipore | ECM625 | USA |
| Invitro angiogenesis | R&D systems | D2300B | USA |
| assay kit | | | |
| Human IL-23 ELISA kit | R&D systems | M1700 | USA |
| Mouse IL-17 ELISA kit | Qualikems | — | India |
| Curcumin | Gibco | 12800-058 | USA |
| DMEM | Sigma | ES134 | USA |
| EDTA | GIBCO | 16000-044 | USA |
| FBS | Sigma | B-2883 | USA |
| Hoechst 33258 dye | Sigma | P-4338 | USA |
| PEG | Himedia | A001A | India |
| Penicillin/Streptomycin | Merck | 61751905001730 | USA |
| Sodium Chloride | Sigma | T-9284 | USA |
| Triton X-100 | Sigma | T4799 | USA |
| Trypsin | Clearsynth | — | India |
| Anthralin | Enzo Life Sciences | — | USA |
| Betullinic Acid | Sigma | — | USA |
| MTT | Cal Biochem | 554720 | USA |
| Retinoic Acid | USP | 1491332 | USA |
| Paclitaxel | SIGMA | C5275 | USA |
| ConA | R&D systems | 285-1F- | |
| IFN-y | 1001CF | USA | |
| (human recombinant) | | | |
| LPS | Sigma | — | USA |
| RPMI-1640 | Himedia | AL162S | India |
| Cell Proliferation kit II | Roche | 11465015001 | USA |
| (XTT) | | | |
| Human VEGF Quantikine | R&D biosystems | DVE00 | USA |
| ELISA Kit | | | |
| Endothelial Cell Growth | Promo cell | C22010 | Germany |
| Medium without VEGF | | | |
| Endothelial Cell basal | Promo cell | C22215 | Germany |
| medium Phenol red free | | | |
| Vascular Endothelial | SIGMA-Aldrich | V7259 | USA |
| Growth Factor human | | | |
| (VEGF) | | | |
| SuperSignal ™ West Pico | Thermo scientific | 34580 | USA |
| PLUS Chemiluminescent | | | |
| Substrate | | | |
| Anti-Mouse IgG (H + L) | Thermo scientific | 32430 | USA |
| Secondary Antibody, HRP | | | |
| Anti-Rabbit IgG (H + L) | Thermo scientific | 32460 | USA |
| Secondary Antibody, | | | |
| HRP | | | |
| Corning ® Transwell ® | SIGMA | CLS3422- | USA |
| polycarbonate membrane | | 48EA | |
| cell culture inserts 6.5 mm | | | |
| Transwell with 8.0 μm | | | |
| pore polycarbonate | | | |
| membrane insert, TC- | | | |
| treated, w/lid, sterile, | | | |
| 48/cs*2 | | | |
| Corning ® Matrigel ® | Corning | 354230 | USA |
| Growth Factor Reduced | | | |
| (GFR) Basement | | | |
| Membrane Matrix, | | | |
| *LDEV-Free, 10 mL | | | |
| PVDF Membrane 30 cm | SIGMA | 3010040001 | USA |
| X 3M | | | |
| Bevizumab | Roche | | USA |
| VEGFR-1 | Cell Signaling | 2893S | USA |
| | Technology (CST) | | |
| VEGFR-2 | Cell Signaling | 9698S | USA |
| | Technology | | |
| VEGFR-3 | Cell Signaling | 2638S | USA |
| | Technology | | |
| NRP-1 | Cell Signaling | 3725S | USA |
| | Technology | | |
| MPO | Cell Signaling | 14569S | USA |
| | Technology | | |
| Akt | Cell Signaling | — | |
| | Technology | | |
| ERK1/2 | | USA | |
| MAPK | Cell Signaling | 4695S | USA |
| | Technology | | |
| p-38 | Cell Signaling | 3042S | USA |
| | Technology | | |
| P-VEGFR-1 | Cell Signaling | 8690S | USA |
| | Technology | | |
| P-VEGFR-2 | SIGMA | SAB4504006 | USA |
| P-VEGFR-3 | Cell Signaling | 2478S | USA |
| | Technology (CST) | | |
| P-Akt | Cell Applications Inc | CY1115 | USA |
| P-ERK1/2 | Cell Signaling | 9271S | USA |
| | Technology | | |
| P-p-38 | Cell Signaling | 4370S | USA |
| | Technology | | |
| P-NRP-1 | Cell Signaling | 4511S | USA |
| | Technology | | |
| GAPDH | Abcam | ab25998 | UK |
| CoC12 | SIGMA | G9545 | USA |
| EDTA | Merck | 102382 | USA |
| FBS | Sigma | E5134 | USA |
| Human IL-1-13 | GIBCO | 16000-044 | USA |
| Human TNF-α | R&D systems | 201-LB/CF | USA |
| Human IFN-γ | R&D systems | 210-TA-005/CF | USA |
| Human IL-20 ELISA kit | R&D systems | 285-1F-100/CF | USA |
| LPS | R&D systems | DL200 | USA |
| Penicillin/Streptomycin | Sigma | L4516 | USA |
| PMA | Himedia | A001A | India |
| RPMI-1640 | Sigma | P-8139 | USA |
| Agarose | Himedia | AL162S | India |
| Doxorubicin | Sigma | A9539 | USA |
| Hydrochloride | | | |
| Ethidium bromide dye | Clearsynth | CS-DR-595 | USA |
| Glacial Acetic acid | Sigma | E8751 | USA |
| Nalidixic acid | BDH | 10001 | USA |
| Topoisomerase II assay | Sigma | N8878 | USA |
| kit | | | |
| Tris | TopoGen | TG1001-1 | USA |
| Human Topoisomerase II | Merck | ME6M560411 | USA |
| enzyme | | | |
| Acetonitrile | TopoGen | TG2000H-1 | USA |
| Methanol | SD fine chemicals | H14A/1714/19 | India |
| | | 08/53 | |
| K₂HPO₄ | SD fine chemicals | E13A/0413/14 | India |
| | | 05/53 | |
| KH₂PO₄ | Sigma-Aldrich | WXBB4178V | USA |
| Solution A | Sigma-Aldrich | SZBD2740V | USA |
| Solution B | BD Biosciences | 18331 | USA |
| HLM | BD Biosciences | 3098857 | USA |
| Propylene glycol | Xenotech | 1210153 | USA |
| Glycerol | Sigma-Aldrich | Lot No. 71150 | USA |
| Tween 20 | Fischer Scientific | | |
| 0.9% NaCI | (24505) | — | USA |
| Trifluoroacetic acid | Sigma | 103K0150 | USA |
| KOH | Axa Parenterals Ltd. | DJ30212 | India |
| Na₂HPO₄ | Spectrochem Pvt. Ltd. | 3302269 | India |
| Imiquimod Cream | NA | Glenmark | USA |
| (Imiquad ®) | | Pharmaceuticals | |
| Veet Cream | NA | Reckitt | India |
| | | Benckiser India | |
| | | Pvt Ltd | |
| BCA Protein Kit Pierce | NA | Thermo | USA |
| | | Scientific, | |
| | | Pierce | |
| Mouse IL-4 ELISA Kit | SM4000B | R & D Sys. | USA |
| Mouse TNF-α ELISA kit | SMTA00B | R & D Sys. | USA |
| Mouse IL-6 ELISA kit | SM6000B | R & D Sys. | USA |
| Mouse IL-17 ELISA kit | SM1700 | R & D Sys. | USA |
| Mouse IL-23 ELISA kit | M2300 | R & D Sys. | USA |
| Mouse IL-13 | DY413 | R & D Sys. | USA |
| Mouse VEGF A ELISA | ab119565 | abcam | UK |
| kit | | | |
| Mouse MPO ELISA Kit | KDM0996 | Kinesis | USA |
| Phorbol 12-myristate 13- | P8139-1MG | Sigma life | USA |
| acetate (TPA) | | sciences, | |
| Dimethyl | 67-68-5 | Merck | India |
| sulfoxide(DMSO) | | SpecialtiesPvt | |
| | | Ltd., | |
| Methanol | MO275 | RANKEM Fine | India |
| | | Chemicals Ltd. | |

### REFERENCES

1. Lizzul P F, Aphale A, Malaviya R, Sun Y, Masud 5, Dombrovskiy V, Gottlieb A B. Differential expression of phosphorylated NF-kappaB/RelA in normal and psoriatic epidermis and downregulation of NF-kappaB in response to treatment with etanercept. J Invest Dermatol. 2005 June; 124(6):1275-83. PubMed PMID: 15955104.
2. Kastelan M, Prpić-Massari L, Brajac I. Apoptosis in psoriasis. Acta Dermatovenerol Croat. 2009; 17(3): 182-6. Review. PubMed PMID: 19818217.
3. Müller K, Prinz H. Antipsoriatic anthrones with modulated redox properties. 4. Synthesis and biological activity of novel 9,10-dihydro-1,8-dihydroxy-9-oxo-2-anthracenecarboxylic and -hydroxamic acids. J Med Chem. 1997 Aug. 15; 40(17):2780-7. PubMed PMID: 9276024.
4. Caspase 3 inactivation to suppress Fas-mediated apoptosis: identification of binding domain with p21 and ILP and inactivation machinery by p21, Atsushi Suzuki1,a, Yumi Tsutomi1, Masayuki Miura2 and Kouichi Akahane3
5. McGill A, Frank A, Emmett N, Turnbull D M, Birch-Machin M A, Reynolds N J. The anti-psoriatic drug anthralin accumulates in keratinocyte mitochondria, dissipates mitochondrial membrane potential, and induces apoptosis through a pathway dependent on respiratory competent mitochondria. FASEB J. 2005 Jun.; 19(8):1012-4. Epub 2005 Mar. 31. PubMed PMID: 15802490.
6. Silva M T. Secondary necrosis: the natural outcome of the complete apoptotic program. FEBS Lett. 2010 Nov. 19; 584(22):4491-9. doi: 10.1016/j.febslet.2010.10.046. Epub 2010 October 26. Review. PubMed PMID: 20974143.
7. http://www.biotek.com/resources/articles/dna-hoechst-33258.html
8. http://www.fasebj.org/content/19/8/1012.full.pdf
9. https://www.researchgate.net/publication/1459907_Quantitation_of_DNA_Fragnentation in_Apoptosis.
10. Kyrylkova K, Kyryachenko S, Leid M, Kioussi C. Detection of apoptosis by TUNEL assay. Methods Mol Biol. 2012; 887:41-7. doi: 10.1007/978-1-61779-860-3_5. PubMed PMID: 22566045.
11. Tino galgon, Stephen Riemschender, AND W.Worlab. Methodological comparison of important apoptosis markers M30 CytoDEATH, Annexin V, and APO2. 2000, BIOCHEMICA, 3:22-24.
12. Detmar M, Brown L F & Claffey K P et al. Overexpression of vascular permeability factor/vascular endothelial growth factor and its receptors in psoriasis. J Exp Med180: 1141-1146.
13. Corticotropin-releasing hormone attenuates vascular endothelial growth factor release from human HaCaT keratinocytes.
14. Anti-angiogenic, Antioxidant, and Anti-carcinogenic Properties of A Novel Anthocyanin-Rich Berry Extract Formula. http://protein.bio.msu.ru/biokhimiya/contents/y69/pdf/bcm_0075.pdf
15. Harant H, Wolff B, Schreiner E P, Oberhauser B, Hofer L, Lettner N, Maier S, de Vries J E, Lindley I J. Inhibition of vascular endothelial growth factor cotranslational translocation by the cyclopeptolide CAM741. Mol Pharmacol. 2007 June; 71(6):1657-65. Epub 2007 Mar. 16. PubMed PMID: 17369307.
16. Reynoso-Roldán A, Roldán M L, Cancino-Diaz J C, Rodriguez-Martinez S, Cancino-Diaz M E. Vascular endothelial growth factor production is induced by histone deacetylase 1 and suppressed by von Hippel-Lindau protein in HaCaT cells. Clin Invest Med. 2012 Dec. 1; 35(6):E340-50. PubMed PMID: 23217559.
17. Komorowski J, Jerczyńska H, Siejka A, Barańska P, Ławnicka H, PawŁowska Z, Stepień H. Effect of thalidomide affecting VEGF secretion, cell migration, adhesion and capillary tube formation of human endothelial EA.hy 926 cells. Life Sci. 2006 Apr. 25; 78(22):2558-63. Epub 2005 Nov. 28. PubMed PMID: 16310808.
18. McGill A, Frank A, Emmett N, Turnbull D M, Birch-Machin M A, Reynolds N J. The anti-psoriatic drug anthralin accumulates in keratinocyte mitochondria, dissipates mitochondrial membrane potential, and induces apoptosis through a pathway dependent on respiratory competent mitochondria. FASEB J. 2005 Jun.; 19(8):1012-4. Epub 2005 Mar. 31. PubMed PMID: 15802490.
19. Ioannou Y A, Chen F W. Quantitation of DNA fragmentation in apoptosis. Nucleic Acids Research. 1996; 24(5):992-993.
20. http://www.ncbi.nlm.nih.gov/pubmed/24765175
21. Novel Anti-Angiogenic peptides derived from ELR-containing CXC chemokines, https://www.researchgate.net/publication/5545607
22. http://www.sciencedirect.com/science/article/pii/S002432050501129X
23. http://www.ncbi.nlm.nih.gov/pubmed/12479700
24. http://www.ncbi.nlm.nih.gov/pubmed/9816139
25. Cornelia Halin et al. (2008) Inhibition of Chronic and Acute Skin Inflammation by Treatment with a Vascular Endothelial Growth Factor Receptor Tyrosine Kinase Inhibitor. Am J Pathol, 173:265-277; DOI: 10.2353/ajpath.2008.071074.
26. Ana B. Cerezo et al. (2017) Inhibition of VEGF-Induced VEGFR-2 Activation and HUVEC Migration by Melatonin and Other Bioactive Indolic Compounds. Nutrients. 9, 249; doi:10.3390/nu9030249.
27. Folkman J (2006). Angiogenesis. Annu Rev Med, 57:1-18.
28. Bainbridge J et al. (2006) Angiogenesis as a therapeutic target in arthritis: lessons from oncology. Curr Pharm Des, 12:2631-2644.
29. Danese S et al. (2006) Angiogenesis as a novel component of inflammatory bowel disease pathogenesis. Gastroenterology, 130:2060-2073.
30. Leong T T et al. (2005) Angiogenesis in psoriasis and psoriatic arthritis: clues to disease pathogenesis. Curr Rheumatol Rep, 7:325-329.
31. Detmar et al. (1994) Overexpression of vascular permeability factor vascular endothelial growth factor and its receptors in psoriasis. J Exp Med, 180:1141-1146.
32. Kunstfeld R et al. (2004) Induction of cutaneous delayed-type hypersensitivity reactions In VEGF-A transgenic mice results in chronic skin inflammation associated with persistent lymphatic hyperplasia. Blood, 104:1048-1057.
33. Ferrara N et al. (2003) The biology of VEGF and its receptors. Nat Med, 9:669-676.
34. Ferrara, N (2005) Angiogenesis as a therapeutic target. Nature, 438, 967-974.
35. Giles, F. J. et al. (2001) the vascular endothelial growth factor (VEGF) signaling pathway: A therapeutic target in patients with hematologic malignancies. Oncologist, 6, 32-39.
36. Ferrara N The biology of vascular endothelial growth factor. Endocr Rev; 18(1): 4-25.
37. Friis, T. et al. (2003) A quantitative ELISA-based co-culture angiogenesis and cell proliferation assay. Apmis, 111(6), 658-668.
38. Yaning Wang et al. (2004) Biological activity of bevacizumab, a humanized anti-VEGF antibody in vitro. Angiogenesis 7: 335-345.
39. Liu Y et al (1995). Hypoxia regulates vascular endothelial growth factor gene expression in endothelial cells: Identification of a 5′ enhancer. Circ Res; 77(3): 638-43.
40. Warren R S et al (1996). Induction of vascular endothelial growth factor by insulin-like growth factor in colorectal carcinoma. J Biol Chem; 271(46): 29483-8.
41. Simon Gerhardt et al (2015).Dimethylfumarate protects against TNF-α-induced secretion of inflammatory cytokines in human endothelial cells. Journal of Inflammation: 12:49.
42. Lawlor et al (2001). PKB/Akt: a key mediator of cell proliferation, survival and insulin responses. J Cell Sci; 114:2903-2910.
43. Shiojima I et al (2002) Role of Akt signaling in vascular homeostasis and angiogenesis. Circ Res; 90:1243-1250.
44. Holmes, K. et al (2007) Vascular endothelial growth factor receptor-2: Structure, function, intracellular signalling and therapeutic inhibition. Cell Signal 2007, 19, 2003-2012.
45. Capitán-Cañadas F, et al (2014) Prebiotic oligosaccharides directly modulate proinflammatory cytokine production in monocytes via activation of TLR4. Mol Nutr Food Res. 2014 May; 58(5):1098-110. doi: 10.1002/mnfr.201300497. Epub 2013 December 23.PubMed PMID: 24549946.
46. Kamata M, et al (2013) Ciclosporin A inhibits production of interleukin-12/23p40 and interleukin-23 by the human monocyte cell line, THP-1.Clin Exp Dermatol. 2013 July; 38(5):545-8. doi: 10.1111/ced.12110. PubMed PMID:23777496
47. Hu Y, Shen F, et al (2010),The IL-17 pathway as a major therapeutic target in autoimmune diseases. Ann N Y Acad Sci. 2011 January; 1217:60-76. Review. PubMed
48. Suzuki E, et al (2014) The IL-23/IL-17 axis in psoriatic arthritis. Autoimmun Rev. 2014 Apr.-May; 13(4-5):496-502.Review. PubMed PMID: 24424175;
49. Bartlett B L, et al (2008) IL-12/IL-23 inhibitors: the advantages and disadvantages of this novel approach for the treatment of psoriasis. Skin Therapy Lett. 2008 Nov.-Dec.; 13(8):1-4. PubMed PMID: 19145382.
50. Wolk K, et al (2009) The Th17 cytokine IL-22 induces IL-20 production in keratinocytes: a novel immunological cascade with potential relevance in psoriasis. Eur J Immunol. 2009 December; 39(12):3570-81. doi: 10.1002/eji.200939687. PubMed PMID: 19830738.
51. Kunz S, et al (2006) Interleukin (IL)-19, IL-20 and IL-24 are produced by and act on keratinocytes and are distinct from classical ILs. Exp Dermatol. 2006 Dec.; 15 (12):991-1004. Pub Med PMID: 17083366.
52. Chen W Y, Chang M S. IL-20 is regulated by hypoxia-inducible factor and up-regulated after experimental ischemic stroke. J Immunol. 2009 Apr. 15; 182(8):5003-12. doi: 10.4049/jimmunol.0803653. PubMed PMID: 19342680
53. Ben-Bassat H. Biological activity of tyrosine kinase inhibitors: novel agents for psoriasis therapy. Curr Opin Investig Drugs. 2001 Nov.; 2(11):1539-45. Review. PubMed PMID: 11763154.
54. Ben-Bassat H, Klein B Y. Inhibitors of tyrosine kinases in the treatment of psoriasis. Curr Pharm Des. 2000 Jun.; 6(9):933-42. Review. PubMed PMID: 10828317.
55. Kwatra S G, Dabade T S, Gustafson C J, Feldman S R. JAK inhibitors in psoriasis: a promising new treatment modality. J Drugs Dermatol. 2012 August; 11(8):913-8. Review. PubMed PMID: 22859235.
56. Mavropoulos A, Rigopoulou E I, Liaskos C, Bogdanos D P, Sakkas L I. The role of p38 MAPK in the aetiopathogenesis of psoriasis and psoriatic arthritis. Clin Dev Immunol. 2013; 2013:569751. doi: 10.1155/2013/569751. Epub 2013 Sep. 12. Review.
57. Soegaard-Madsen L, Johansen C, Iversen L, Kragballe K. Adalimumab therapy rapidly inhibits p38 mitogen-activated protein kinase activity in lesional psoriatic skin preceding clinical improvement. Br J Dermatol. 2010 June; 162(6):1216-23. doi: 10.1111/j.1365-2133.2010.09706.x. Epub 2010 Mar. 25. PubMed PMID: 20346019.
58. Bidyottam Mittra et al. Luteolin, an abundant dietary component is a potent anti-leishmanial agent that acts by inducing Topoisomerase II mediated Kinetoplast DNA Cleavage leading to apoptosis. Molecular Medicine; 6, 527-541, 2000.
59. Topoisomerase II assay kit (Plasmid based), Catalogue No. TG1001-1, TopoGen.
60. B S Dawrkanath et al. Inhibitors of topoisomerases as anticancer drugs: problems and prospects. Indian journal of experimental biology; 42, 649-659, 2004.
61. Devander singh. Topoisomerases II inhibitors in cancer treatment. International Journal of Pharmaceutical science and general nanotechnology; 3; 1173-1181, 2011.
62. World Health Organisation-WHO Technical Report Series, No. 953. 2009. Annex-2 Stability testing of active Pharmaceutical ingredients and finished Pharmaceutical products.
63. Schedule Y, http://cdsco.nic.in/html/schedule-y %20(amended %2version-2005)%2orignal.htm
64. International Conference on Harmonization (2003) Q1A (R2): Stability testing of new drug substances and products (second revision).
65. International Conference on Harmonization (2003) Q1E: Evaluation of stability data.

### IS217 Molecule as Anti-Psoriatic Drug has at Least the Following Advantages:

IS217 is a non-biologic, non-steroidal, synthetic small peptide of 10 amino acids. IS217 has dual mechanism of action targeting VEGF and IL-17/IL-23 and thus offers benefits of anti-angiogenesis and anti-inflammatory action. The best efficacy offered by current biologics target IL-17 or IL-23 pathway hence IS217 has the potential to improve on this as well the oral drugs targeting the angiogenesis and inflammatory pathways. Cost of production for the peptide-based therapy is likely to be less versus biologics. Thus, it offers room for competitive pricing and gets a larger market share in both SC and topical forms. IS217 has potential to meet large unmet need in topical formulations. There are no targeted topical formulations and topical IS217 could lead in the class by promising improved efficacy and limit the risk of unwanted toxicities.

IS217 molecule drug developments for psoriasis: To develop IS217 as an anti-psoriatic drug, the in vitro mechanistic studies in HUVEC cell lines proved IS217 as anti-VEGF molecule and anti-inflammatory activity was proved by using splenocytes and human monocytes THP-1. In vivo studies were completed in Balb/c mice to evaluate anti-psoriatic potential of IS217 by subcutaneous and topical route of administration in IMQ induced psoriasis model.

Conclusions: The present invention provides IS217 molecule for drug development for prevention and treatment of psoriasis. From the overall studies, it could be concluded that IS217 used in their respective doses, exhibit remarkable potential in efficaciously attenuating the symptoms and processes underlying psoriasis-like dermatitis in mice. Since IS217, being a lytic synthetic peptide having anti-angiolytic activity and anti-inflammatory properties that could antagonize the deleterious effects of the pro inflammatory mediators responsible for the onset of the chronic disease, they may therefore prove to have potential in preventing, delaying and curing the disease and can open the way to new therapeutic strategies for psoriasis treatment.

However, though IMQ-induced psoriasis in mice share the cytokine profile and histopathological findings to those of psoriatic lesions in humans, further research is required and underway to validate that they are corresponding disease expressions. The current findings on psoriasis-like mice model could be found appropriate on the human psoriatic model only after such validations and further clinical research. Furthermore, further research is under way for elucidation of elaborate mechanisms followed by IS217 in regulating internal factors for effectively dealing with the disease.

The experimental studies carried in present invention establish that it is possible to treat inflammatory skin diseases such as psoriasis by subcutaneous and/or topical administration of an anti-angiogenic and anti-inflammatory IS217 molecule and formulation or combination thereof.

Although the subject matter has been described herein with reference to certain preferred embodiments thereof, other embodiments are possible. The invention may be embodied in other specific forms without departing from the spirit or essential characteristics thereof. The foregoing embodiments are therefore to be considered in all respects illustrative rather than limiting the invention described herein. It will be obvious to those skilled in the art to make various changes, modifications and alterations to the invention described herein. To the extent that these various changes, modifications and alteration do not depart from the scope of the present invention, they are intended to be encompassed therein. Scope of the invention is thus indicated by the appended claims rather than by the foregoing description, and all changes that come within the meaning and range of equivalency of the claims are intended to be embraced therein.

1. A stable anti-angiogenic and anti-inflammatory pharmaceutical formulation for treating and/or preventing one or more symptoms of psoriasis, said formulation comprising a peptide of SEQ. ID NO 1 or peptide variant thereof in an amount of from 0.01 μg/ml to 10,000 μg/ml and one or more suitable pharmaceutically acceptable excipients.

2. The formulation as claimed in claim 1, wherein said one or more suitable pharmaceutically acceptable excipients are selected from the group consisting of suitable carriers, diluents, vehicles, disintegrant, swelling agent, antioxidant, buffer, bacteriostatic agent, emollient, emulsifier, plasticizer, penetration enhancer, preservative, cryoprotectant, neutralizer, fragrance additives, dispersants, surfactants, binders and lubricants.

3. The formulation of claim 1, wherein said peptide variant is at least 80%, at least 85%, at least 90%, at least 95%, at least 97%, at least 99% identical to the SEQ. ID NO 1.

4. The formulation of claim 1, wherein said formulation is suitable for use as an injectable formulation, preferably by subcutaneous route of administration and/or topical mode of administration.

5. (canceled)

6. A stable anti-angiogenic and anti-inflammatory pharmaceutical combination, said combination comprising a peptide of SEQ. ID NO 1 or peptide variant thereof in an amount from 0.01 μg/ml to 10,000 μg/ml and one or more other active agent.

7. (canceled)

8. The combination of claim 6, wherein said other active agent is selected from the group consisting of anthralin, betamethasone and methotrexate.

9. The combination of claim 6, wherein said one or more other active agent is in an amount of 0.01 μM to 50 μM.

10. The combination of claim 6, wherein said peptide variant is at least 80%, at least 85%, at least 90%, at least 95%, at least 97%, at least 99% identical to the SEQ. ID NO 1.

11. The combination of claim 6, wherein said combination is suitable as an injectable formulation, preferably by subcutaneous route of administration and/or topical mode of administration.

12. (canceled)

13. A method of preparing stable anti-angiogenic and anti-inflammatory pharmaceutical formulation comprising the steps:
a) preparing an aqueous phase with required quantity of pharmaceutically acceptable excipients by suitable means and heating the aqueous phase to a suitable temperature;
b) preparing an oil phase with required quantity of pharmaceutically acceptable excipients by suitable means and heating the oil phase to a suitable temperature until a clear solution is obtained;
c) preparing a solution comprising peptide of SEQ. ID NO 1 or peptide variant thereof with required quantity of pharmaceutically acceptable excipients by suitable means;
d) adding oil phase obtained in step ‘b’ to aqueous phase obtained in step ‘a’ under continuous stirring for about 30 minutes and continuously increasing the stirring speed when both the phases are at same temperature;
e) cooling solution obtained in step ‘d’ to a suitable temperature followed by addition of solution obtained in step ‘c’ under continuous stirring at increased stirring speed;
f) adding required quantity of tea tree oil in the solution obtained in step ‘e’ under continuous stirring and adding water until a clear homogenous cream is formed.

14. The method as claimed in claim 13, further comprising packaging of said cream prepared in suitable containers and optionally comprising sterilization of said cream by suitable sterilization methods before or after packaging of said cream in suitable containers.

15. (canceled)

16. The method of claim 13,
wherein at least one of the following conditions is present:
(ii) said suitable temperature in steps ‘a’ and ‘b’ is 70° C. to 80° C.;
(ii) said suitable temperature in step ‘e’ is 30° C. to 40° C.;
(iii) steps ‘a’ and ‘b’ require stirring at about 600 rpm;
(iv) said increasing the stirring speed in step ‘d’ is an increase to about 1000 rpm: or
(v) said increased stirring speed in step ‘e’ is about 2000 rpm.

17-20. (canceled)

21. The method of claim 13, wherein at least one of the following is present:
(i) said pharmaceutically acceptable excipients in step ‘a’ comprises glycerol and/or triethanolamine;
(ii) said pharmaceutically acceptable excipients in step ‘b’ comprises cetyl alcohol, stearic acid, beeswax, isopropyl myristate and/or mineral oil; or
(iii) said pharmaceutically acceptable excipients in step ‘c’ comprises ethylenediamine tetra-acetic acid disodium salt and/or trehalose dihydrate.

22-23. (canceled)

24. A method of preventing and inhibiting angiogenesis in a subject in need thereof, said method comprising administering to the subject a therapeutically effective amount of a formulation or a combination comprising a therapeutically effective amount of peptide of SEQ. ID NO 1 or peptide variant thereof wherein the formulation or combination inhibits vascular endothelial growth factor (VEGF) expression.

25. The method as claimed in claim 24, wherein said therapeutically effective amount of peptide of SEQ. ID NO 1 or peptide variant thereof in said formulation or combination is 0.01 μg/ml to 10,000 μg/ml and optionally, said combination comprises one or more other active agents selected from the group consisting of anthralin, betamethasone and methotrexate in an amount of 0.01 μM to 50 μM.

26. (canceled)

27. The method of claim 24, wherein said formulation or said combination is administered to the subject as an injectable formulation, preferably by subcutaneous mode of administration and/or topical mode of administration.

28. A method of preventing and inhibiting inflammation in a subject in need thereof, said method comprising administering to the subject a therapeutically effective amount of the formulation of claim 1, wherein said formulation inhibits expression of one or more of inflammatory markers p38 kinases, IL-17, IL-23 and IL-20.

29. The method as claimed in claim 28, wherein said therapeutically effective amount of peptide of SEQ. ID NO 1 or peptide variant thereof in said formulation is 0.01 μg/ml to 10,000 μg/ml, and optionally said formulation comprises one or more other active agent selected from a group consisting of anthralin, betamethasone and methotrexate in an amount of 0.01 μM to 50 μM.

30. (canceled)

31. The method of claim 28, wherein said formulation is administered to the subject as an injectable formulation, preferably by subcutaneous mode of administration and/or topical mode of administration.

32-34. (canceled)

35. A method of manufacturing decapeptide of SEQ. ID NO 1 or peptide variant thereof comprising the steps:
a) synthesizing said decapeptide by coupling one amino acid at a time, starting from C-terminus amino acid which is attached to a solid resin via a linker group;
b) controlling coupling of step a) by varying de-protection time and reagents, wherein de-protection is performed twice;
c) drying and weighing peptide resin obtained after coupling last amino acid;
d) cleaving resin-bound peptide off said resin by trifluoroacetic acid to obtain crude peptide;
e) optionally processing crude peptide obtained in step d) by reverse phase chromatography and ion exchange to obtain solution of purified peptide;
f) optionally lyophilizing said solution of purified peptide for removal of residual solvents.


SEQUENCE LISTING (1 sequence)

SEQ ID NO: 1 - protein, 10 aa, Artificial Sequence
  Synthetic peptide IS217
FAKKFAKKFK